(19) United States
(12) Patent Application Publication
(10) Pub. No.: US 2024/0279629 A1
(43) Pub. Date: Aug. 22, 2024

(54) CRISPR-TRANSPOSON SYSTEMS FOR DNA MODIFICATION

(71) Applicant: The Trustees of Columbia University in the City of New York, New York, NY (US)

(72) Inventors: Samuel Henry Sternberg, New York, NY (US); George Davis Lampe, New York, NY (US); Rebeca Teresa King Davidson, New York, NY (US); Alejandro Chavez, New York, NY (US); Sanne Eveline Klompe, New York, NY (US)

(21) Appl. No.: 18/567,617

(22) PCT Filed: Jun. 7, 2022
(86) PCT No.: PCT/US22/32541

§ 371 Date: Dec. 6, 2023

Related U.S. Application Data
(60) Provisional application No. 63/284,837, filed on Dec. 1, 2021.
(60) Provisional application No. 63/236,337, filed on Aug. 24, 2021.
(60) Provisional application No. 63/211,631, filed on Jun. 17, 2021.
(60) Provisional application No. 63/197,889, filed on Jun. 7, 2021.

(51) Int. Cl. C12N 9/22; C07K 14/195; C12N 15/11; C12N 15/63; C12N 15/90

(52) U.S. Cl. CPC C12N 9/22; C07K 14/195; C12N 15/111; C12N 15/63; C12N 15/90; C07K 2319/09; C12N 2310/20

(57) ABSTRACT

The present disclosure provides systems, kits, and methods for nucleic acid integration utilizing engineered Clustered Regularly Interspaced Short Palindromic Repeats (CRISPR)-CRISPR-associated transposon (CRISPR-Tn) system. More particularly, the present disclosure provides systems comprising: an engineered CRISPR-Tn system or one or more nucleic acids encoding the engineered CRISPR-Tn system, wherein the CRISPR-Tn system comprises at least one or both of: a) at least one Cas protein (e.g., Cas6, Cas7, Cas5, and/or Cas8); and b) one or more transposon-associated proteins (e.g., TnsA, TnsB, TnsC, TnsD, and/or TniQ). The present disclosure also provides systems, kits, and methods for nucleic acid integration in a eukaryotic cell.

### STATEMENT REGARDING FEDERALLY-SPONSORED RESEARCH OR DEVELOPMENT

This invention was made with government support under grant number HG011650 awarded by the National Institutes of Health. The government has certain rights in the invention.

### FIELD

The present invention relates to methods and systems for DNA modification and gene targeting comprising engineered Clustered Regularly Interspaced Short Palindromic Repeats (CRISPR)-associated transposon (CRISPR-Tn) system. Particularly, the present invention relates to methods and systems for RNA-guided DNA integration comprising engineered CRISPR-associated transposon systems.

### CROSS-REFERENCE TO RELATED APPLICATIONS

This application claims the benefit of U.S. Provisional Application Nos. 63/197,889, filed Jun. 7, 2021, 63/211,631, filed Jun. 17, 2021, 63/236,337, filed Aug. 24, 2021, and 63/284,837, filed Dec. 1, 2021, the contents of each of which are herein incorporated by reference in their entirety.

### SEQUENCE LISTING STATEMENT

The text of the computer readable sequence listing filed herewith, titled “39595-601_SEQUENCE_LISTING_ST25”, created Jun. 7, 2022, having a file size of 1,992,779 bytes, is hereby incorporated by reference in its entirety.

### BACKGROUND

CRISPR-Cas systems are prokaryotic immune systems that confer resistance to foreign genetic elements such as plasmids and bacteriophages. The canonical CRISPR/Cas9 system exploits RNA-guided DNA-binding and sequence-specific cleavage of a target DNA. A guide RNA (gRNA) is complementary to a target DNA sequence upstream of a PAM (protospacer adjacent motif) site. The Cas (CRISPR-associated) 9 protein binds to the gRNA and the target DNA, and introduces a double-strand break (DSB) in a defined location upstream of the PAM site. The ability of the CRISPR-Cas9 system to be programmed to cleave not only viral DNA but also other genes opened a new venue for genome engineering.

The past decade has revealed an astounding diversity of CRISPR-Cas systems that utilize RNA guides for sequence-specific nucleic acid targeting, thereby providing host organisms with adaptive immunity against invading mobile genetic elements (MGEs). CRISPR-Cas systems are currently grouped into two classes (1-2), six types (I-VI) and dozens of subtypes, depending on the signature and accessory genes that accompany the CRISPR array. Although RNA-guided targeting typically leads to endonucleolytic cleavage of the bound substrate, recent studies have uncovered a range of noncanonical pathways in which CRISPR protein-RNA effector complexes have been naturally repurposed for alternative functions.

### SUMMARY

Provided herein are systems, kits, and methods that facilitate nucleic acid editing, particularly systems, kits, and methods that facilitate RNA-guided nucleic acid integration

Provided herein are systems for DNA integration into a target nucleic acid sequence comprising: an engineered Clustered Regularly Interspaced Short Palindromic Repeats (CRISPR)-CRISPR associated (Cas) transposon (CRISPR-Tn) system or one or more nucleic acids encoding the engineered CRISPR-Tn system, wherein the CRISPR-Tn system comprises at least one or both of: a) at least one Cas protein; and b) one or more transposon-associated proteins.

In some embodiments, each of the at least one Cas protein and one or more of the at least one transposon-associated protein are part of a single fusion protein.

The systems or kits may further comprise c) at least one gRNA (gRNA) or a nucleic acid encoding a gRNA, wherein the at least one gRNA is complementary to at least a portion of a target nucleic acid sequence. In some embodiments, the at least one gRNA is a non-naturally occurring gRNA. In some embodiments, the at least one gRNA is encoded in a CRISPR RNA (crRNA) array. In some embodiments, the at least one gRNA is transcribed under control of an RNA Polymerase II or an RNA Polymerase III promoter.

In some embodiments one or more of the at least one Cas protein are part of a$ ribonucleoprotein complex with the gRNA.

In some embodiments, the at least one Cas protein is derived from a Type I CRISPR-Cas system (e.g., Type I-F, Type I-B). In some embodiments, the at least one Cas protein comprises Cas5, Cas6, Cas7, and Cas8. In some embodiments, the at least one Cas protein comprises Cas8-Cas5 fusion protein.

In some embodiments, the at least one transposon protein is derived from a Tn7 or Tn7-like transposon system. In some embodiments, the at least one transposon-associated protein comprises TnsB and TnsC. In some embodiments, the at least one transposon-associated protein comprises TnsA, TnsB, and TnsC.

In some embodiments, the at least one transposon protein comprises a InsA-TnsB fusion protein. In some embodiments, the TnsA-TnsB fusion protein further comprises an amino acid linker between TnsA and InsB. The linker may be a flexible linker. In some embodiments, the linker comprises at least one glycine-rich region. In some embodiments, the linker comprises a NLS sequence. In some embodiments, the linker comprises a NLS sequence flanked on each end by a glycine rich region.

In some embodiments, the at least one transposon-associated protein comprises TnsD and/or TniQ.

In some embodiments, the CRISPR-Tn system is derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

In some embodiments, one or more of the at least one Cas protein and the at least one transposon-associated protein comprises a nuclear localization signal (NLS). In some embodiments, one or more of the at least one Cas protein and the at least one transposon-associated protein comprises two or more NLSs. In some embodiments, the NLS is appended to the one or more of the at least one Cas protein and the at least one transposon-associated protein at a N-terminus, a C-terminus, or a combination thereof.

The NLS may be a monopartite sequence or a bipartite sequence. In some embodiments, the NLS comprises a sequence having at least 70% similarity to KRTADGSEFESPKKKRKV (SEQ ID NO:89).

In some embodiments, the one or more nucleic acids comprises one or more messenger RNAs, one or more vectors, or a combination thereof.

In some embodiments, the at least one Cas protein, the at least one transposon-associated protein, and the gRNA are encoded by different nucleic acids.

In some embodiments, one or more of the at least one Cas protein, the at least one transposon-associated protein, and the gRNA are encoded by a single nucleic acid.

In certain embodiments, Cas7 is encoded by an individual nucleic acid. In certain embodiments, Cas7 or the nucleic acid encoding Cas7 is in greater abundance compared to the remaining protein components or nucleic acids encoding thereof.

In some embodiments, a single nucleic acid encodes the gRNA and at least one Cas protein (e.g., Cas6 or Cas7).

In some embodiments, each of the at least one Cas protein, the at least one transposon-associated protein, and the gRNA are encoded by a single nucleic acid.

In some embodiments, the one or more nucleic acids further comprises or encodes a sequence capable of forming a triple helix downstream of the sequence encoding the at least one Cas protein or the sequence encoding the at least one transposon-associated protein. In some embodiments, the sequence capable of forming a triple helix is in a 3′ untranslated region of the sequence encoding the at least one Cas protein or the sequence encoding the at least one transposon-associated protein.

In some embodiments, one or more of the nucleic acids encoding at least one Cas protein and the nucleic acids encoding the at least one transposon-associated protein comprises a sequence encoding a ribosome skipping peptide. In some embodiments, the ribosome skipping peptide comprises a 2A family peptide.

In some embodiments, the systems further comprise a donor nucleic acid to be integrated, wherein said donor DNA comprises a cargo nucleic acid sequence flanked by at least one transposon end sequence.

Additionally, provided herein are systems for DNA integration into a target nucleic acid sequence comprising: an engineered Clustered Regularly Interspaced Short Palindromic Repeats (CRISPR)-CRISPR associated (Cas) transposon (CRISPR-Tn) system or one or more nucleic acids encoding the engineered CRISPR-Tn system, wherein the CRISPR-Tn system comprises at least one or both of: a) at least one Cas protein; and b) TnsA, TnsB, TnsC, or a combination thereof. In some embodiments, the engineered CRISPR-Tn system is derived from Vibrio parahaemolyticus, Aliibrio sp., Pseudoalteromonas sp., or Endozoicomonas ascidiicola. In some embodiments, the engineered CRISPR-Tn system is a Type I-F system (e.g., a Type I-F3 system).

In some embodiments, the one or more nucleic acids comprises one or more messenger RNAs, one or more vectors, or a combination thereof.

In some embodiments, wherein the one or more nucleic acids further comprise or encode a sequence capable of forming a triple helix downstream of the sequence encoding the engineered CRISPR-Tn system. In some embodiments, the sequence capable of forming a triple helix is in a 3′ untranslated region of the sequence encoding the at least one Cas protein or the sequence encoding at least one of TnsA, TnsB, TnsC, TnsD, and TniQ.

In some embodiments, one or more of the nucleic acids encoding the engineered CRISPR-Tn system comprises a sequence encoding a ribosome skipping peptide. In some embodiments, the ribosome skipping peptide comprises a 2A family peptide.

In some embodiments, the at least one Cas protein and the TnsA, TnsB, and TnsC are encoded by different nucleic acids. In some embodiments, the at least one Cas protein and the TnsA, TnsB, and TnsC are encoded by a single nucleic acid.

In some embodiments, the at least one Cas protein comprises Cas5, Cas6, Cas7, and Cas8. In some embodiments, the at least one Cas protein comprises Cas8-Cas5 fusion protein. In certain embodiments, Cas7 or the nucleic acid encoding Cas7 is in greater abundance compared to the remaining protein components or nucleic acids encoding thereof.

In some embodiments, the engineered CRISPR-Tn system further comprises TnsD, TniQ, or a combination thereof or a nucleic acid encoding TnsD, TniQ, or a combination thereof.

In some embodiments, the engineered CRISPR-Tn system comprises Cas5, Cas6, Cas7, Cas8, TnsA, TnsB, TnsC, and at least one or both of TnsD or TniQ. In some embodiments, the engineered CRISPR-Tn system comprises TnsA, TnsB, TnsC, TnsD and TniQ.

In some embodiments, one or more of the at least one Cas protein, TnsA, TnsB, TasC, TnsD, and TniQ comprises a nuclear localization signal (NLS). In some embodiments, one or more of the at least one Cas protein, TnsA, TnsB, TnsC, TnsD, and TniQ comprises two or more NLSs. In some embodiments, the NLS is appended to the one or more of the at least one Cas protein, TnsA, TnsB, TnsC, TnsD, and TniQ at a N-terminus, a C-terminus, or a combination thereof.

In some embodiments, TnsA and InsB are provided as a TnsA-TnsB fusion protein. In some embodiments, the TnsA-TnsB fusion protein further comprises an amino acid linker between TnsA and TnsB. In some embodiments, the linker is a flexible linker. In some embodiments, the linker comprises at least one glycine-rich region.

In some embodiments, the linker comprises a nuclear localization signal (NLS). In some embodiments, the linker comprises a NLS flanked on each end by a glycine rich region.

In some embodiments, the NLS is a monopartite sequence. In some embodiments, the NLS is a bipartite sequence. In some embodiments, the NLS comprises a sequence having at least 70% similarity to KRTADGSEFESPKKKRKV (SEQ ID NO:89).

In some embodiments, the engineered CRISPR-Tn system further comprises a gRNA (also referred to herein as CRISPR RNA, or crRNA) complementary to at least a portion of the target nucleic acid sequence, or a nucleic acid encoding the at least one gRNA. In some embodiments, the at least one gRNA is encoded by a nucleic acid different from the nucleic acid(s) encoding the at least one Cas protein and TnsA, TnsB, and TnsC. In some embodiments, the at least one gRNA is encoded by a nucleic acid also encoding the at least one Cas protein, TnsA, TnsB, and TnsC, or both.

In some embodiments, the at least one gRNA is a non-naturally occurring gRNA. In some embodiments, the at least one gRNA is encoded in a CRISPR RNA (crRNA) array.

In some embodiments, the system further comprises a target nucleic acid sequence. In some embodiments, the target nucleic acid sequence comprises a human sequence. In some embodiments, the target nucleic acid sequence comprises a TnsD binding site.

In some embodiments, the systems further comprise a donor nucleic acid flanked by at least one transposon end sequence. In some embodiments, the donor nucleic acid comprises a human nucleic acid sequence. In some embodiments, the nucleic acid encoding the at least one Cas protein, TnsA, TnsB, and TnsC, the at least one gRNA, or any combination thereof further comprises the donor nucleic acid.

In some embodiments, the system is a cell-free system.

In addition, compositions comprising the disclosed systems are provided herein.

Also provided are cells comprising the disclosed systems. In some embodiments, the cell is a prokaryotic cell. In some embodiments, the cell is a eukaryotic cell (e.g., a mammalian cell or a human cell).

Further disclosed are methods for DNA integration comprising contacting a target nucleic acid sequence with a system or a composition disclosed herein.

In some embodiments, the target nucleic acid sequence is in a cell. In some embodiments, contacting a target nucleic acid sequence comprises introducing the system into the cell. In some embodiments, the cell is a prokaryotic cell. In some embodiments, the cell is a eukaryotic cell (e.g., a mammalian cell or a human cell).

In some embodiments, introducing the system into the cell comprises administering the system to a subject. In some embodiments, administering comprises in vivo administration. In some embodiments, administering comprises transplantation of ex vivo treated cells comprising the system.

Kits comprising any or all of the components of the systems described herein are also provided. In some embodiments, the kit further comprises one or more reagent, shipping and/or packaging containers, one or more buffers, a delivery device, instructions, or a combination thereof.

Other aspects and embodiments of the disclosure will be apparent in light of the following detailed description.

### BRIEF DESCRIPTION OF THE DRAWINGS

FIGS. 1A-1E show RNA-guided transposition activity of type I-F3 CRISPR-Tn. FIG. 1A is the genomic layout of Tn6677 (V. cholerae INTEGRATE). The machinery required for transposon mobilization can be functionally divided into the transposition module that facilitates excision and integration of the transposon (TnsA-TnsB) through interactions with a regulator protein (TnsC), and a DNA-targeting module that identifies the site for integration. Type I-F CRISPR-Tn use the RNA-guided DNA-binding complex TniQ-Cascade (crRNA₁Cas8₁Cas7₆Cas6₁ TniQ₂) for target site determination. L, left end; R, right end. FIG. 1B is an overview of selected Type I-F3 CRISPR-Tn systems. Location refers to the host gene found adjacent to the right end of transposon, which provides a target for the atypical crRNA homing pathway; no atypical homing crRNA was found for Tn7017/parE, marked with an *. FIG. 1C is a schematic representation of a transposition assay in which a mini-Tn is targeted to a site in the E. coli genome and detected via junction PCR. FIG. 1D is a graph of the integration efficiency for all the systems at 37° C., measured by qPCR. ND, not detected. FIG. 1E is a graph of the integration efficiency for Tn7017at 25° C. and 37° C., measured by qPCR. ND, not detected. Data in FIGS. 1D and 1E are shown as mean±s.d. for n=3 biologically independent samples.

FIGS. 2A-2D show the PAM requirements and integration site variation for CRISPR-Tn systems. FIG. 2A is a schematic representation of a PAM library in which a pTarget plasmid encodes a 32-bp target sequence flanked by a 5-bp degenerate sequence. FIG. 2B is violin plots of PAM enrichment for Tn6999 (Type V-K CRISPR-Tn, ShoINT) and Tn7016. Lines represent 10-fold enrichment or depletion. *, PAM sequences not detected in the final library. FIG. 2C is WebLogos of top 5% enriched PAM sequences and integration site distribution obtained from the PAM library data for Tn7016 and Tn6999. d, distance in bp from the 3′ end of the target to the transposon. FIG. 2D is a graph of integration efficiencies for TN7016 and PAMs indicated, normalized to a ‘CC’ PAM. Data are shown as mean±s.d. for n=3 biologically independent samples.

FIGS. 3A-3D show Tn7017 exploits distinct TniQ homologs for two different targeting pathways. FIG. 3A is a schematic representation of Tn7017, showing the presence of two distinct ThiQ/TnsD genes. FIG. 3B is a pruned phylogenetic tree of TniQ/TnsD with different Tn7-like transposons and CRISPR-Tn systems (I-B1, I-B2, and I-F3) indicated. ‘TniQ’ and ‘TnsD’ are used to describe TiQ/TnsD proteins involved in the RNA-guided or protein-mediated homing pathway, respectively Two clades of I-F3-TnsD proteins are shown, the darker hue indicates the putative homing TosD proteins described in Petassi et al. (Cell 183, 1757-1771.e18), while the lighter color clade includes TnsD from Tn7017. FIG. 3C is a transposition assay design for simultaneous detection of DNA integration at a genomic target site (RNA-guided) and a putative, plasmid-borne homing site (RNA-independent). FIG. 3D is a graph of integration efficiency for pTarget and the genomic target site, as measured by qPCR, under different gene deletion conditions. Data are shown as mean±s.d. for n=3 biologically independent samples.

FIG. 4A is a schematic of a pooled library approach to determine cross-reactivity between protein-RNA machinery and the mini-transposon DNA. FIG. 4B is a graph of relative integration efficiency for Tn7016, tested in a strain with or without a pre-existing mini-Tn6677, measured by qPCR. These data demonstrate that orthogonal CRISPR-Tn systems can be used for high-efficiency tandem insertions of genetic payloads.

FIGS. 5A-5F show transposition activity of type I-F3 CRISPR-Tn under different conditions. FIG. 5A is a graph of integration efficiency for the systems as indicated using the crRNA and temperature conditions shown, measured by qPCR. FIG. 5B shows possible mini-Tn integration orientations (top right), and the observed bias (tRL:tLR) for each CRISPR-Tn system under the temperature conditions shown, determined from qPCR measurements (bottom left). Integration orientation data may be skewed for low efficiency systems because of detection limitations. FIG. 5C is a layout of typical (dark grey diamonds) and atypical (light grey diamonds) repeats within the native CRISPR array(s). Atypical spacers (light grey squares) and their target genes (yciA, ffs, and rsm.J) are indicated. The bracketed number indicates the length of the atypical spacer. FIG. 5D is consensus logos of the safe harbor loci targeted by atypical spacers for the systems. The atypical guide RNAs targeting these sites are indicated above the consensus logos with flipped-out bases (light grey) and mismatched bases (dark grey) indicated in bars above the sequence. FIG. 5E is consensus logos of typical and atypical repeats, revealing loss of conservation for the last 8 bp of the atypical repeats. FIG. 5F is a graph of the integration efficiency as determined by qPCR for 32 bp spacers with atypical repeats.

FIGS. 6A-6D show PAM requirements and integration site variation. FIG. 6A is violin plots displaying the enrichment of PAM variants as a result of RNA-guided transposition for different CRISPR-Tn systems. CRISPR-Tn with <0.05% integration activity is masked in grey since their activity may have bottlenecked PAM representation. FIGS. 6B and 6C are WebLogos for the top (FIG. 6B) or bottom (FIG. 6C) 5% enriched PAM sequences per CRISPR-Tn system. The base positions are numbered from the protospacer start, with −1 representing the base immediately adjacent to the protospacer. Low sequence conservation represents the absence of sequence restraints and therefore more flexible PAM requirements. CRISPR-Tn with <0.05% integration activity is masked in grey since their activity may have bottlenecked PAM representation. FIG. 6D is a graph of integration site distribution for ‘CC’ PAMs obtained from the PAM library dataset. Systems with >0.5% total integration efficiency at 37° C. are shown. The distance from target site is the number of bases between the terminal base of the protospacer and the first base of the transposon sequence (and therefore includes the 5-bp target site duplication). Orange indicates a distance of 49-bp away, which is the primary integration site for many of the CRISPR-Tn.

FIG. 7A is a comparison of predicted protein domains of EcoTnsD (Tn7), EasTnsD (Tn7017), and EasTniQ (Tn7017). Predicted TniQ (PF06527) and TnsD (PF15978) domains from InterProScan analysis are shown. FIG. 7B is integration efficiency at the genomic protospacer with or without pTarget present, under different gene deletion environments.

FIG. 8 is a schematic of the genomic layout and cargo analysis of native CRISPR-transposons. CRISPR-Tn systems encode multiple cargo genes in addition to the transposition and CRISPR-Cas operons. The native genomic layout of CRISPR-transposon in this study is shown, and putative defense systems are indicated based on pfam.

FIG. 9 is a table of homologous CRISPR-transposon systems. The table describes CRISPR-Tn systems described herein. Each system may be alternately referred to by a dedicated Tn identifier (Tn #), a homolog identifier (Homolog #), the organism from which the transposon derives, and/or a simplified ID that derives from the organism name. Mini-transposon donor DNA substrates and expression vectors encoding the protein-RNA machinery from each system are designed and constructed using sequence information derived from the transposon.

FIG. 10A is a vector map of a pcDNA3.1 derivative plasmid, with a representative depiction of a cas6 gene under CMV promoter control, with N-terminal nuclear localization signal (NLS) and 3×FLAG epitope tags. pA, polyadenylation signal. FIG. 10B is Western blots for various Cas6 constructs. The ID shown correlates to FIG. 9. (−) represents the native DNA sequence for each Cas6 species; (+) refers to human codon optimization of the cas6 gene sequence. Beta-actin was stained as a loading control.

FIGS. 11A-11E show a GFP repression assay to assess guide RNA processing by Cas6. FIG. 11A shows an exemplary plasmid design for Cas6 expression and Direct-Repeat (DR) GFP reporter plasmids within a pcDNA3.1-derivative expression vector. The DR for Vch is shown (SEQ ID NO: 295), as well as the Cas6 cleavage site (red arrow). FIG. 11B is a schematic of the GFP repression assay. When the DR-GFP plasmid is transfected alone, successful transcription and translation of GFP occurs, leading to elevated levels of GFP fluorescence as measured by flow cytometry. The stem loop within the Direct Repeat is formed in the 5′ UTR, downstream of the 5′ cap (red circle). When a plasmid encoding the cognate Cas6 is co-transfected, Cas6 binds to the stem loop in the 5′-UTR and cleaves the mRNA. This leads to loss of the 5′ cap, RNA degradation, and a loss of GFP fluorescence. FIG. 11C is representative raw flow cytometry data for Cas6 and its cognate DR from a canonical Type I-F1 CRISPR-Cas system derived from Pseudomonas aeruginosa (Pae), or from the Type I-F3 CRISPR-Cas system derived from the Vibrio cholerae HE-45 CRISPR-Tn system (Tn6677, Vch). Cells were transfected with either the DR-GFP plasmid alone (left), or the DR-GFP plasmid together with Cas6 expression plasmid (right). In the presence of Cas6, a severe reduction in GFP fluorescence is observed. FIG. 11D is a bar graph showing relative GFP mean fluorescence intensity (MFI) for the GFP repression assay using various Cas6 homologs and different fusion constructs. Cas6 tags such as NLSs were appended either N-terminally (e.g., NLS-Cas6) or C-terminally (e.g., Cas6-NLS). Data were normalized to the DR-GFP only control. FIG. 11E is a bar graph of relative GFP MFI for additional Cas6 homologs, denoted belong the graph. The numbers above each bar within FIGS. 11D-11E represent experimental identifiers that correspond to the information described in Table 3.

FIGS. 12A-12E show the tdTomato activation assay to assess transposon DNA binding by TnsB. FIG. 12A is a schematic and sequence of right (SEQ ID NO: 297) and left (SEQ ID NO: 296) transposon ends derived from V. cholerae Tn6677 (e.g., VchINTEGRATE). Putative TnsB binding sites are highlighted in blue boxes (top) and represented by blue arrows (bottom). FIG. 12B is an exemplary plasmid design for TnsB-NLS-VP64 activator construct within a pcDNA3.1-derivative expression vector. FIG. 12C is a schematic of the activation assay. A reporter plasmid contains a minimal CMV promoter, a tdTomato expression cassette, and a CRISPR-transposon end. Two orientations of the right end shown in FIG. 12A were tested. When transfected alone, the reporter minimally expresses tdTomato. When a plasmid expressing TnsB-VP64 is co-transfected, it binds to the transposon end, leading to elevated levels of tdTomato expression. FIG. 12D is a bar graph showing tdTomato activation for various tdTomato reporter plasmids with VchTnsB-VP64. The negative control represents a plasmid that did not contain a transposon end inserted upstream of the minimal CMV promoter. The only substantive transcriptional activation is observed with the RE Fwd Reporter when co-transfected with the TnsB-bpNLS-VP64 construct. TdTomato MFI is plotted relative to experimental ID 27. FIG. 12E is a bar graph showing tdTomato activation for additional TnsB homologs. The numbers above each bar within FIGS. 12D-12E represent experimental identifiers that correspond to the information described in Table 3.

FIGS. 13A-13F show development and characterization of a InsAB fusion polypeptide. FIG. 13A is a schematic of fusion of InsA and TnsB leading to a single InsAB polypeptide. FIG. 13B is a graph of the E. coli integration efficiency of Vch INTEGRATE (derived from Tn6677) with various tags appended to TnsA and/or TnsB. N-terminal NLS tagging of TnsA, and C-terminal 2A tagging of TnsB, both lead to severe reductions in integration. Efficiencies are shown for both tRL and tLR orientation products, and are normalized to the WT system. FIG. 13C is a schematic of an exemplary engineered TnsAB fusion containing an internal BP NLS (SEQ ID NO: 89) and glycine-serine linkers (L) (SEQ ID NO: 298). The inset (below) shows the primary amino acid sequence (positions 224-266 of SEQ ID NO: 96) for the insertion, color coded as in the top diagram. FIG. 13D is a graph of the E. coli integration efficiency for various TnsA-TnsB fusion (InsABf) constructs, in which various NLS tags were placed either N-terminally, C-terminally, or internally. The internal bpNLS tag, as schematized in FIG. 13C, has even higher activity than WT TnsA+TnsB. FIG. 13E is HEK293T Western Blot data for TnsA(bpNLS)B_{f} protein, after nuclear and cytoplasm fractionation. HDAC1 was used as a nuclear-specific control, and alpha-tubulin was used as a cytoplasmic-specific control. These data demonstrate efficient expression of the full-length fusion polypeptide. FIG. 13F is TdTomato transcriptional activation using TnsAB_{f}, applying methods described in FIG. 12. The numbers above each bar within FIGS. 13B, 13D, and 13F represent experimental identifiers that correspond to the information described in Table 3.

FIGS. 14A-14C show a plasmid-to-plasmid transposition assay to reconstitute human cell RNA-guided DNA integration activity with VchINTEGRATE. FIG. 14A is a schematic of exemplary pDonor and pTarget plasmids used to reconstitute plasmid-to-plasmid RNA-guided DNA integration in HEK293T cells; the integrated pTarget product DNA is shown at the right. The relevant origins of replication, antibiotic resistance markers, and mini-transposon (Mini-Tn), are shown. The sequence targeted by the gRNA encoded on pSL2084 is represented with a maroon rectangle, and the PAM is shown in yellow. Genes and other regulator components are not shown to scale. FIG. 14B is a schematic of the overall strategy, in which pDonor, pTarget, and protein/gRNA expression plasmids are used to co-transfect HEK293T cells, allowing for RNA-guided DNA integration to proceed during the 48-72 growth post-transfection. Plasmid DNA is then purified from the cell population and used to transform E. coli NEB 10-beta cells. Notably, pDonor is unable to replicate in this cell strain, such that chloramphenicol-resistant (CmR+) colonies are only expected to arise from the successful transposition of the mini-Tn (encoding CmR) to pTarget. FIG. 14C is a table of plasmids that are used to co-transfect HEK293T cells in these experiments, with a simplified plasmid name (left), a brief description of the plasmid function (right), and a numeric ID associated with the specific plasmid (middle). The sequence of each plasmid, according to this ID, is described in Tables 4-7. Control experiments with a non-targeting gRNA utilized pSL1409 in place of pSL2084.

FIGS. 15A-15C show the genotypic analysis of human-cell RNA-guided DNA integration products. FIG. 15A is a schematic of PCR strategy used to amplify integration products from chloramphenicol-resistant E. coli transformants with pTarget containing the site-specifically inserted mini-transposon DNA that was originally encoded on pDonor. FIG. 15B is agarose gel electrophoresis of colony PCR products using the strategy shown in FIG. 15A. The lanes indicated with * show clear evidence of an amplicon around 460 bp in length, consistent with the expected amplicon size from the integrated pTarget product DNA. The lane marked “L” represents a 100 bp DNA ladder (GoldBio); lanes marked “NT” (non-targeting) used background CmR+ colonies from plasmid mixtures that were derived from HEK293T cells transfected with a non-targeting gRNA plasmid. FIG. 15C is Sanger sequencing analysis confirms the presence of a bona fide integration product, in which the mini-transposon is inserted 49-bp downstream of the 3′ edge of the target site, as depicted in the schematic aligned to the sequencing chromatograms. Comparison of sequencing products derived from both novel junctions between the pTarget and the mini-transposon (mini-Tn) clearly indicates the presence of the expected 5-bp target-site duplication (TSD), highlighted in purple. SEQ ID NO: 299, top Sanger sequence analysis, SEQ ID NO: 300, lower Sanger sequence analysis.

FIGS. 16A and 16B show that modified gRNA expression cassettes retain potent RNA-guided DNA targeting activity. FIG. 16A is schematic of an exemplary initial gRNA expression strategy (top) employing a separate plasmid encoding the gRNA as a repeat-spacer-repeat array, controlled by a human U6 promoter, and a modified pDonor plasmid (bottom) in which the CRISPR array expression cassette is placed just downstream of the mini-transposon. FIG. 16B is a graph of QCascade and TnsC-VP64 transcriptional activation using the modified gRNA expression plasmids, in which the gRNA was encoded on pDonor itself. The levels of activation, as measured by relative mCherry MFI (normalized to the non-targeting control) are nearly indistinguishable between the initial gRNA expression strategy (FIG. 16A, top) and the modified strategy in which the gRNA is encoded on pDonor (FIG. 16A, bottom). The numbers above each bar in FIG. 16B represent experimental identifiers that correspond to the information described in Table 3.

FIGS. 17A-17C show RNA Polymerase II-based expression of guide RNAs for VchINTEGRATE. FIG. 17A is schematics of different methods to express the gRNA. The CRISPR array (repeat-spacer-repeat) is canonically encoded on an RNA Pol III promoter (e.g., human U6), such that the nascent transcript stays primarily nuclear. However, it can also be encoded within the 3′-UTR of an RNA Pol II transcript, alongside the use of features such as the MALAT1 triplex to stabilize upstream protein-coding transcripts after cleavage. Cleavage occurs upon repeat-spacer-repeat processing by the Cas6 ribonuclease subunit of Cascade. FIG. 17B is schematic of the various constructs generated and tested within a pcDNA3.1-derivative expression vector. The MALAT1 triplex and CRISPR array were inserted into the 3′-UTR of either VchCas6 or VchCas7. FIG. 17C is a bar graph showing transcriptional activation data using constructs described in FIG. 17B. These results demonstrate that Pol II-encoded gRNAs are functional for RNA-guided DNA targeting and TnsC-based activation above background, defined here as the non-targeting gRNA control. The numbers above each bar in FIG. 17C represent experimental identifiers that correspond to the information described in Table 3.

FIGS. 18A-18B show TnsC-based transcriptional activation as a method to screen homologous CRISPR-Tn systems in human cells. FIG. 18A is a schematic of the transcriptional activation assay. When transfected alone, the mCherry reporter minimally expresses mCherry because it is controlled by a minimal CMV promoter. When plasmids expressing QCascade, TnsC-VP64, and a gRNA that recognizes the target present on the reporter plasmid are co-transfected, QCascade (blue oval) binds to the target sequence and recruits TnsC-VP64 (light orange ovals), leading to elevated levels of mCherry expression Three copies of TosC-VP64 are shown for simplicity to demonstrate the oligomeric nature of TnsC recruitment; the actual number of TnsC proteins that are recruited to target sites in cells may be significantly larger. FIG. 18B is a bar graph showing mCherry activation with various homologous CRISPR-Tn systems. An enlarged graph in which Tn6677 is omitted is included (right panel). Data were measured by flow cytometry, and the cellular mCherry mean fluorescence intensity (MFI) was plotted relative to the non-targeting gRNA control for each system. The numbers above each bar within panel B represent experimental identifiers that correspond to the information described in Table 9.

FIGS. 19A-19B show plasmid-to-plasmid transposition assay to reconstituted human cell RNA-guided DNA integration activity with VchINTEGRATE. FIG. 19A is a schematic of the overall strategy, in which pDonor, pTarget, and protein/gRNA expression plasmids are used to co-transfect HEK293T cells, allowing for RNA-guided DNA integration to proceed during the 48-72 growth post-transfection. HEK293T cell DNA is then harvested, and two sequential rounds of PCR are performed; “nested” primers (shown in green) are used in the second PCR to heighten sensitivity. The first round of PCR was performed with oSL5946 and oSL5169, and the second, “nested” round of PCR was performed with oSL5947 and oSLS072. FIG. 19B is agarose gel electrophoresis of PCRs performed on DNA extract from cells that were co-transfected with all necessary Tn7016 components, and either a scrambled gRNA (NT gRNA, pSL2917), or a gRNA that recognizes pTarget (T gRNA, pSL2918), are shown. The expected amplicon representing a junction sequence is marked by a green box, and was purified for additional analysis.

FIGS. 20A-20D show quantitative analysis of Tn7016 integration activity and successful truncation of transposon ends in human cells. FIG. 20A is a graph of quantitative real-time qPCR data to quantify integration efficiency for Tn7016 in HEK293T cells, using either a targeting (T) or non-targeting (NT) gRNA. Integration efficiency was calculated as a comparison of amplification of the junction amplicon compared to a segment of pTarget that would not contain a junction sequence. oSL5946 and oSL6032 were used to amplify integration events, while oSL5010 and oSL5011 were used to amplify a separate region of pTarget. FIG. 20B is a schematic showing Tn7016 transposon ends and putative TnsB binding sites Below, the lengths of DNA sequence that were cloned into pDonor plasmids, derived from the Pseudoalteromonas sp. S983 genome, is indicated. pDonor plasmid IDs used in bacterial integration assays are denoted on the left. Note that the sequence regions used to not correspond to the minimal transposon end sequences; for example, in the case of pSL2190, 250-bp starting from both ends of the Pseudoalteromonas genomic Tn7016 were used, despite encompassing the requisite features for transposase recognition plus additional sequence corresponding to the cargo of the native transposon. Subsequent designs (pSL3591, pSL3592, pSL3593) shorted the left end to 145-bp and the right end to the indicated lengths (150-bp, 75-bp, and 57-bp). FIG. 20C is a graph of bacterial transposition assays to identify active truncated variants of the right end of the Tn7016 Mini-Tn. A non-targeting (NT) negative control was included. The different length base pair (bp) descriptions define the length of the right end of Tn7016 in each experimental sample. Similarly designed pDonor plasmids, but specifically for human-cell plasmid-to-plasmid transposition assays, were subsequently designed and tested. Plasmid descriptions can be found in Table 8. FIG. 20D is quantitative real-time qPCR data to quantify integration efficiency for Tn6677 and Tn7016 in HEK293T cells. The newly designed truncated Mini-Tn for Tn7016 was used in order for the same primer pair to be used to amplify both Tn6677 and Tn7016 insertion events. Integration efficiency was calculated as a comparison of amplification of the junction amplicon compared to a segment of pTarget that would not contain a junction sequence. oSL5946 and oSL5950 were used to amplify integration events, while oSL5010 and oSL5011 were used to amplify a separate region of pTarget. The numbers above each bar within FIGS. 20A, 20C, and 20D represent experimental identifiers that correspond to the transformation/transfection information described in Table 9.

FIG. 21 is a graph of the impact of NLS placement on various components of Tn7016. Using a plasmid-to-plasmid RNA-guided DNA integration assay in human cells, the placement of bipartite nuclear localization signals (NLS) was varied on the protein components shown in the bottom of the figure; note that the TnsABf fusion protein contains an internal NLS and was not altered in any of these experiments. In the first condition on the left (19), all shown protein components contained an N-terminal NLS tag (‘N’). In subsequent experiments (20-25), the NLS tag was moved from the N-terminus to the C-terminus for the indicated protein(s). Transfections were initially performed such that each transfection contained one Tn7016 component in which the N-terminal NIS tag was repositioned to the C-terminus; a final transfection was performed (25) such that all Tn7016 components other than TnsABf possessed a C-terminal NLS tag. All integration efficiencies are normalized to a transfection in which cells were transfected with all requisite components with listed NLS locations and a targeting gRNA The numbers above each bar represent experimental identifiers that correspond to the transfection information described in Table 9.

FIGS. 22A-22E show reconstitution of protein-RNA INTEGRATE components in human cells. FIG. 22A is a schematic detailing DNA integration using RNA-guided transposases. FIG. 22B are schematics of Type I-F CRISPR-associated transposons that encode the CRISPR RNA and seven proteins for DNA integration (top). Mammalian expression vectors used for heterologous reconstitution in human cells are shown at bottom. FIG. 22C are Western blots with anti-FLAG antibody demonstrating robust protein expression upon individual (−) or multi-plasmid (+) co-transfection of HEK293T cells. Co-transfections contained all VchINT components, with the FLAG-tagged subunit(s) indicated. β-actin was used as a loading control. FIG. 22D is a schematic of eGFP knockdown assay to monitor crRNA processing by Caso in HEK293T cells. Cleavage of the CRISPR direct repeat (DR)-encoded stem-loop severs the 5′-cap from the ORF and polyA (pA) tail, leading to a loss of eGFP fluorescence (bottom). FIG. 22E is a graph of transposon-encoded VchCas6 (Type I-F3) RNA cleavage and eGFP knockdown, as measured by flow cytometry. Knockdown was comparable to PseCas6 from a canonical CRISPR-Cas system (Type I-E), was absent with a non-cognate DR substrate, and was sensitive to C-terminal tagging. To control for over-expression artifacts, data were normalized to negative control conditions (−), in which dCas9 was co-transfected with the reporter. Data are shown as mean±s.d. for n=3 biologically independent samples.

FIGS. 23A-23H show RNA-guided DNA integration in human cells using diverse CRISPR-associated transposases. FIG. 23A shows the initial detection of bona fide transposition products by colony PCR analysis, after plasmids were isolated from human cells and selected in E. coli (left). A positive amplicon selected for additional analysis is marked with a red asterisk, and Sanger confirmed the expected insertion site position and presence of target-site duplication (right). FIG. 23B is a phylogenetic tree of Type I-F3 CRISPR-associated transposon systems, with labels indicating the homologs that were tested in human cells. FIG. 23C is a comparison of plasmid-to-plasmid integration efficiencies with VchINT (Tn6677) and PseINT (Tn7016), as measured by qPCR. FIG. 23D shows amplicon sequencing reveals a strong preference for integration 49-bp downstream of the 3′ edge of the site targeted by the crRNA. FIG. 23E shows optimization of PseINT integration efficiency by varying NLS placement and plasmid stoichiometries, as measured by qPCR. Unless otherwise noted, all components contained an NLS tag on the N terminus of the protein, or internally in the case of pTnsAB_{f}. TniQ-NLS indicates a TniQ construct in which the placement of the NLS tag was changed from the N terminus to the C terminus of the protein. TnsC-NLS and TnsC-3×NLS indicate TnsC constructs in which the placement of either 1 NLS or 3 NLS tags was changed from the N terminus to the C terminus of the protein. Plasmid amounts transfected are detailed in nanograms (ng). pTniQ-NLS, pTnsC-NLS, and pTnsC-3×NLS were transfected in 100 ng amounts, unless otherwise stated. FIG. 23F is a graph of deletion experiments confirming the contribution of each protein component, a targeting crRNA, and intact transposase active site (D220N mutation in TnsB, D458N mutation in TnsAB_{f}) for successful integration. FIG. 23G is a graph of RNA-guided DNA integration with genetic payloads spanning 1-15 kb in size, transfected based on molar amount, as determined by qPCR. FIG. 23H is graph of RNA-guided DNA integration showing a strong sensitivity to mismatches across the entire 32-bp target site. Data were measured by qPCR and normalized to the perfectly matching (PM) crRNA. Data in FIG. 23D are shown as mean n=2 biologically independent samples. Data in FIGS. 23C and 23E-H are shown as mean±s.d. for n=3 biologically independent samples.

FIGS. 24A-24D show expression and nuclear localization of VchINT components. FIG. 24A is Western blotting of various VchINT components using distinct nuclear localization signals (NLS). Each component was appended with a 3×FLAG epitope tag and NLS tag, and nuclear fractionation was performed to separate nuclear and cytoplasmic cellular proteins. Histone deacetylase 1 (HDAC1) and α-Tubulin were used as nuclear- and cytoplasmic-specific loading controls, respectively. FIG. 24B are schematics of multiple exemplary fusions designs of TnsA and TnsB (TnsAB_{f}), with an NLS appended internally or at the N- or C-terminus. FIG. 24C is a graph of RNA-guided DNA integration activity determined in E. coli with the indicated TnsABt variants, as measured by qPCR. FIG. 24D is Western blotting of TnsAB_{f} with internal NLS validating expression and nuclear localization. The observed band was at the expected size, with no evidence of degradation or internal cleavage.

FIGS. 25A-25C show initial detection and optimization of targeted integration using VchINT. FIG. 25A shows nested PCR strategy to detect plasmid-transposon junctions directly from HEK293T cell lysates (left), and agarose gel electrophoresis showing target-cargo junction product bands (right). Expected amplicon sizes are marked for each PCR reaction with red arrows, and the crRNA was either non-targeting (NT) or targeting (T). “H₂O” denotes a condition in which the lysate was omitted from the PCR reactions. An aliquot of PCR is used for PCR 2 such that a “nested PCR” is performed. Sanger sequencing was performed on the product after PCR 2 in the targeting condition (bottom right, SEQ ID NO: 303). FIG. 25B is a schematic of Taqman probe strategy used to improve signal-to-noise by selectively detecting novel plasmid-transposon junctions. Probes labeled with FAM (blue) are used to detect target-transposon junctions, and probes labeled with SUN (green) are used to detect the target plasmid backbone, for integration efficiency quantification. Probes that span the junction of pTarget and the right transposon end of VchINT (SEQ ID NO: 304) are designed to anneal to an insertion event 49-bp downstream of the target site. FIG. 25C is a graph of integration efficiencies which were improved by varying the relative levels of pDonor, pTarget, or protein expression plasmids, as indicated; data were measured by qPCR and are normalized to a control sample transfected with 100 ng of each component. Data in FIG. 25C are shown as mean for n=2 biologically independent samples.

FIGS. 26A-26E show systematic screening of homologous Type I-F CRISPR-associated transposons to uncover improved systems for mammalian cell applications. FIG. 26A is a cartoon depicting the multi-tiered approach that was applied to screen the indicated systems through a series of consecutive activity assays, with associated schematics shown for each functional assay. The middle panel depicts a transcriptional activation assay designed to monitor transposon DNA binding by TnsB in human cells using a tdTomato reporter plasmid. FIG. 26B is Western blotting to detect expression of candidate Cas6 homologs in HEK293T cells, with or without human codon optimization (hCO), using anti-FLAG antibody; β-actin was used as a loading control. A range of expression levels for human codon-optimized gene variants was observed, and genes were poorly expressed for most systems when native bacterial coding sequences were used. FIG. 26C is a graph of activity assays for Cas6 homologs using the GFP knockdown assay shown in FIG. 22D. For each homolog, GFP fluorescence levels were measured by flow cytometry and normalized to the experimental condition in which the GFP reporter plasmid lacked a CRISPR direct repeat (DR) in the 5′-UTR. FIG. 26D is transcriptional activation data for TnsB-VP64 constructs from selected homologous CRISPR-associated transposons, as measured by flow cytometry. FIG. 26E is transcriptional activation data for QCascade and TnsC-VP64 from homologous CRISPR-associated transposons, as measured by flow cytometry. Tn7016, the final homolog that was selected for additional screening for transposition, is marked with a red arrow and asterisk. Data in FIGS. 26C-26E are shown as mean for n=2 biologically independent samples.

FIGS. 27A-27G show parameter screening to further improve integration activity with the PseINT (Tn7016) system. FIG. 27A is RNA-guided DNA integration efficiency for TnsAB fusion (TnsAB_{f}) protein design, with or without internal NLS, compared to the wild-type TnsA and TnsB proteins. Experiments were performed in E. coli, and efficiencies were measured by qPCR. FIG. 27B is Tn7016 transposon ends shortened relative to previously tested constructs, generating the constructs indicated with red dashed boxes at the top. RNA-guided DNA integration activity was compared for the indicated variants in E. coli, as measured by qPCR (bottom). The final pDonor design used in FIG. 23 contains 145-bp and 75-bp derived from the native left and right ends of Pseudoalteromonas Tn7016, respectively. FIG. 27C is Agarose gel electrophoresis showing successful junction products from nested PCR (top) for PseINT, and Sanger sequencing chromatograms showing the expected integration distance (bottom; SEQ ID NO: 305). FIG. 27D is integration efficiencies in HEK293T cells were similar using either typical or atypical CRISPR repeats, as measured by qPCR. FIG. 27E is RNA-guided DNA integration activity compared with the indicated BP NLS tags on PseINT components, as measured by qPCR. Individual components had their respective BP NLS tag repositioned from the N- to the C-terminus; “All” represents a condition in which all components had BP NLS tags on the noted terminus. Interestingly, the observed tag sensitivity is similar to, but distinct from, that with VchINT components. Various combinations of N- and C-terminal NLS tagging for PseQCascade and PseTnsC. NT=non-targeting crRNA. Nuclear export signal (NES) predictions for PseINT wild type (WT) and mutant TnsC. A putative NES within TnsC could lead to inefficient nuclear localization, and multiple residues were selected that, when mutated, might lower this risk. Predicted NES sequences were generated using NetNES. FIG. 27F shows RNA-guided DNA integration activity compared after appending additional NLS tags on PseTnsC and removing a potential internal nuclear export signal (NES) sequence. FIG. 27G is RNA-guided DNA integration activity compared after varying the relative levels of individual PseINT protein and RNA expression plasmids. Data were measured by qPCR and are normalized to either a control sample transfected with 100 ng of each component (left), or a control sample transfected with the standard PseINT plasmid amounts, as detailed in the Methods section (right). Data in FIGS. 27A, 27B and 27D are shown as the mean±s.d. for n=3 biologically independent samples. Data in FIGS. 27E, 27G, and 27H are shown as the mean for n=2 biologically independent samples.

FIGS. 28A-28D show selection, seeding, and sorting strategies result in further increases in PseINT integration efficiencies. FIG. 28A is normalized RNA-guided DNA integration efficiency for PseINT in the absence or presence of puromycin selection, and after harvesting cells from between 2-6 days post-transfection. Experiments used a puromycin resistance plasmid as a transfection selection marker, in addition to PseINT component plasmids, and integration activity was measured by qPCR and normalized to the condition harvested on day 3 without puromycin selection. FIG. 28B is PseINT integration efficiencies compared as a function of seeding density 24 hours before transfection. 24-well plates were with various cell densities ranging from 103 to 2×10⁵ cells per well, and integration activity was measured by qPCR. FIG. 28C is a schematic showing the use of a GFP transfection marker and cell sorting to increase integration efficiency. A GFP expression plasmid was transfected in significantly smaller amounts relative to PseINT component plasmids, and cells were sorted into bins of varying GFP expression levels. FIG. 28D show PseINT integration efficiencies are enhanced after using flow cytometry to sort cells for the brightest GFP positive cells. Cells were sorted four days after transfection, and the top 20% brightest cells were binned in increments of 5%, with Bin 1 representing the top 5% brightest cells and Bin 4 representing the 15-20% brightest cells. Integration efficiencies were determined for each bin separately, or for the unsorted population, as measured by qPCR. Integration efficiencies were normalized to the unsorted, targeting crRNA condition. Data in FIG. 28A are shown as the mean of n=2 biologically independent samples. Data in FIGS. 28B and 28D are shown as the mean+s.d. for n=3 biologically independent samples.

FIGS. 29A-29C show PseINT integration is biased towards tRL insertion and reproducibly quantified across distinct approaches. FIG. 29A shows RNA-guided DNA integration is heavily biased towards insertion in the right-left (tRL) orientation, with only a small minority of insertion events occurring in the left-right (tLR) orientation. Integration efficiencies were calculated using SYBR qPCR. FIG. 29B shows the strategy to detect and quantify integration efficiencies using PCR and next-generation sequencing. A variant pDonor was construct, in which a primer binding site is present within the transposon cargo at a distance from the transposon right end (R), such that unintegrated and integrated pTarget molecules yield amplicons of indistinguishable length using pF and pR primers (left). Consequently, next-generation sequencing of these amplicons can provide relative ‘counts’ of edited and unedited alleles in the population, without introduction of PCR bias. Agarose gel electrophoresis demonstrates identical amplicon products for non-targeting (NT) and targeting (T) samples after PCR 1 for NGS analysis (right). FIG. 29C shows calculated integration efficiencies for the same experimental samples, measured by Taqman qPCR, droplet digital PCR (ddPCR), and amplicon deep sequencing. ddPCR and qPCR analyses specifically probe for integration products that are 49-bp downstream of the target site, whereas amplicon sequencing analysis does not impose the same stringent distance bias, allowed the quantification of integration products within a larger window surrounding the anticipated integration site. Editing efficiencies for both PseINT and VchINT were consistent between different quantification methods. Data in FIG. 29A are shown as the mean±s.d. for n=3 biologically independent samples. Data in FIG. 29C are shown as the mean for n=2 biologically independent samples.

FIGS. 30A-30D show RNA-guided DNA integration at endogenous human genomic target sites. FIG. 30A is an exemplary design of amplicon sequencing assay to detect and quantify RNA-guided genomic integration. Transfected pDonor constructs contain an embedded ˜20-nt sequence identical to a genomic region (orange) downstream of a site targeted by a cognate crRNA. After transfection, a PCR reaction is performed with a single pair of primers, in which DNA sequences from both unedited and edited genomic loci can be simultaneously amplified. Next generation sequencing (NGS) is used to differentiate and quantify unedited (wild-type) and edited (integration-positive) alleles. FIG. 30B is a graph demonstrating successful integration into endogenous human genomic target sites using CRISPR-transposon systems. Control transfections delivered a non-targeting gRNA (NT), resulting in zero integration events being detected. However, when a gRNA was used to target the sequence 5′-acagtggggccactagggacaggattggtgac-3′ (SEQ ID NO: 293) within AAVS1 (denoted “T” in the graph, integration events were detected and the frequency of edited alleles relative to wild-type alleles could be quantified. FIG. 30C shows the analysis of the NGS data from experiments presented in FIG. 30B revealing the integration site distribution of detected integration events. Integration events are tallied based on the distance between the end of the 32-nucleotide target sequence and the first nucleotide of the integrated transposon end. The distance distribution is consistent with molecular determinants that have been observed from other experiments performed in human cells and bacterial cells. FIG. 30D is a graph of RNA-guided DNA integration observed at additional endogenous human genomic target sites, as revealed by amplicon sequencing. Shown are data resulting from experiments that targeted one of two target sites in AAVS1, and a third target site present in the ACTB locus.

FIG. 31 is a graph of RNA-guided DNA integration activity using modified guide CRISPR RNAs. The spacer length of CRISPR arrays was varied as shown in the x-axis, and compared with a non-targeting control crRNA that had a spacer length of 32-nt. Within this experiment, the highest integration efficiency was achieved using a spacer length of 33-nt, which is 1-nt longer than the typical spacer length (32-nt; asterisk) that is observed within CRISPR arrays for Type I-F CRISPR-transposon systems.

FIGS. 32A-32C show streamlined polycistronic expression vectors for TniQ-Cascade complex. FIG. 32A shows protein components for PseINT (e.g., derived from Tn7016) tested for their sensitivity to NLS tagging at either their N-termini (“N”) or C-termini (“C”). For bars labeled “All,” the ThiQ, Cas8, Cas7, and Cas6 components all contained the same N- or C-terminal NLS tags. For all other conditions, all components contained an N-terminal NLS tag except for the indicated protein component, which was tagged at the indicated terminus (e.g., C-terminus). The results demonstrate that C-terminal NLS tags on TiQ lead to ablation of integration activity, whereas all of the other protein components (e.g., Cas8, Cas7, and Cas6) are equally active when tagged at their C-termini with NLS tags as when they are tagged at the N-termini with NLS tags. FIG. 32B shows the investigation of polycistronic TniQ-Cascade protein expression vectors via plasmid-to-plasmid integration assays. Given the tolerance of C-terminal NLS tags across all Cascade components for PseINT (derived from Tn7016), several polycistronic vectors were constructed through the placement of NLS tags and 2A peptides, such that all protein components of the TniQ-Cascade complex will be expressed off of a single mRNA transcript. NLS tags were placed directly upstream of the 2A peptide sequences such that Cascade subunits would only have a C-terminal peptide tag. TniQ was always included as the final translated component since it does not tolerate a C-terminal tag. “Separate Vectors” represents a transfection in which all components were expressed on separate pcDNA3.1-like expression vectors driven by a CMV promoter. FIG. 32C shows the investigation of polycistronic TniQ-Cascade protein expression vectors via genomic integration assays, targeting an endogenous AAVS1 target sequence. Further investigation of polycistronic vectors expressing Cas7 at the start of the polycistronic operon revealed increased integration efficiencies when TniQ-Cascade was translated in one particular order (Cas7, Cas8, Cas6, TniQ). “Separate Vectors” represents a transfection in which all components were expressed on separate pcDNA3.1-like expression vectors driven by a CMV promoter.

FIGS. 33A-33C show additional homologous CRISPR-transposon systems for RNA-guided DNA integration. FIG. 33A is a schematic of the constructs used to screen ThiQ homologs for their function in human cells when combined with PseINT components derived from Tn7016. The vectors used in these experiments express Cascade protein components (e.g., Cas7, Cas8, and Cas6) on a polycistronic design using 2A “skipping peptides”, as well as a TnsABt fusion polypeptide, and TnsC, all from Tn7016; not shown are the pCRISPR vector encoding a Tn7016-specific crRNA, the pDonor encoding a Tn7016-specific mini-transposon, and the pTarget used for DNA integration assays. These vectors were combined with a TniQ expression vector, in which the TniQ protein was derived from either Tn7016 (e.g., PseINT) or from a variety of homologous CRISPR-transposon systems as shown in FIG. 33B. Integration efficiencies are measured using plasmid-to-plasmid transposition assays performed in human cells. FIG. 33B shows the sequence similarity of TniQ proteins from the indicated homologous CRISPR-transposon systems, which are close to Tn7016 in terms of evolutionary relatedness. The percent sequence identity at the amino acid level is shown for TiQ from several CRISPR-transposons. FIG. 33C shows RNA-guided integration activity for plasmid-to-plasmid transposition assays, which Tn7016 (e.g., PseINT) components were combined with TniQ homologs from the indicated CRISPR-transposon homolog. The Tn7016 components functioned robustly with the TniQ protein from Tn7018, Tn7019, and Tn7020, whereas the TniQ homologs from Tn7015 and Tn7014 were not able to complement the system. The ΔTniQ control condition lacked any TniQ and showed a complete loss of RNA-guided DNA integration activity, as expected.

### DETAILED DESCRIPTION

The disclosed systems, kits, and methods provide systems and methods for nucleic acid integration utilizing engineered CRISPR-transposon systems. The disclosed systems, kits, and methods provide systems and methods for RNA-guided DNA integration utilizing engineered CRISPR-transposon systems.

Provided herein are transposons derived from bacteria that, in some cases, exhibit nearly PAM-less targeting. High-throughput sequencing and transposon sequence motif analysis identified highly active systems that exhibit orthogonality in transposon DNA recognition and mobilization.

Tn7-like and Tn5053-like transposons that encode nuclease-deficient CRISPR-Cas systems, also known as CRISPR-transposons (CRISPR-Tn), catalyze the Insertion of Transposable Elements by Guide RNA-Assisted TargEting (INTEGRATE). The molecular and sequence determinants of RNA-guided DNA integration for a representative Tn7-like transposase system derived from Vibrio cholerae Tn6677, which encodes a Type I-F CRISPR-Cas system, was previously described (Klompe et al., Nature 571, 219-225 (2019)).

Provided herein are systems, kits, and methods that allow detection and optimization of INTEGRATE reactions in mammalian cells (e.g., human cells), as well as improvements to mammalian expression vectors that yield higher expression and/or improved nuclear trafficking. Also provided herein are engineered and improved InsA-TnsB fusion proteins (referred to as TnsAB_{f}), which are active for RNA-guided transposition and may be used as a substitute for separately encoded InsA and TnsB proteins. Expression vector designs, in which the guide RNA is encoded on an RNA Polymerase II promoter-controlled gene, within the 3′-untranslated region (UTR), allowing guide RNA processing and assembly of the TniQ-Cascade complex in the cytoplasm. Also provided are expression vectors encoding homologous INTEGRATE systems, as well as activity assays for components derived from these homologous INTEGRATE systems.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e g., Braasch and Corey, Biochemistry, 41(14): 4503-4510 (2002)) and U.S. Pat. No. 5,034,506), locked nucleic acid (LNA; see Wahlestedt et al., Proc. Natl. Acad Sci. U.S.A, 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46: 453 (1960) and Doty et al., Proc. Natl. Acad. Sci. USA, 46: 461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### CRISPR-Tn Systems for DNA Integration

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Transcription of a CRISPR locus produces a “pre-crRNA,” which is processed to yield crRNAs containing spacer-repeat fragments that guide effector nuclease complexes to cleave dsDNA sequences complementary to the spacer. Several different types of CRISPR systems are known, (e.g., type I, type II, or type III), and classified based on the Cas protein type and the use of a proto-spacer-adjacent motif (PAM) for selection of proto-spacers in invading DNA.

Although RNA-guided targeting typically leads to endonucleolytic cleavage of the bound substrate, recent studies have uncovered a range of noncanonical pathways in which CRISPR protein-RNA effector complexes have been naturally repurposed for alternative functions. For example, some Type I (Cascade) and Type II (Cas9) systems leverage truncated guide RNAs to achieve potent transcriptional repression without cleavage and other Type I (Cascade) and Type V (Cas12) systems lie inside unusual bacterial Tn7-like transposons and lack nuclease components altogether.

Disclosed herein are systems or kits for DNA integration into a target nucleic acid sequence comprising: an engineered Clustered Regularly Interspaced Short Palindromic Repeats (CRISPR)-CRISPR associated (Cas) transposon (CRISPR-Tn) system or one or more nucleic acids encoding the engineered CRISPR-Tn system, wherein the CRISPR-Tn system comprises at least one or both of: a) at least one Cas protein; and b) one or more transposon-associated proteins.

In some embodiments, the systems or kits may further comprise c) a guide RNA (gRNA) or a nucleic acid encoding a gRNA, wherein the gRNA is complementary to at least a portion of a target nucleic acid sequence. In some embodiments, one or more of the at least one Cas protein are part of asibonucleoprotein complex with the gRNA.

In some embodiments, the engineered CRISPR-Tn system is derived from Vibrio parahaemolyticus, Aliibrio sp., Pseudoalteromonas sp., or Endozoicomonas ascidiicola. In some embodiments, the engineered CRISPR-Tn systems are derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

In some embodiments, the system comprises components from different CRISPR-Tn systems. In some embodiments, one or more of the at least one Cas protein and one or more transposon-associated proteins may be derived from a homologous CRISPR-transposon system compared to the other protein components in the system. Thus, in some embodiments, one or more of the components of the engineered CRISPR-Tn system is derived from Vibrio parahaemolyticus, Alibrio sp., Pseudoalteromonas sp., or Endozoicomonas ascidiicola. In some embodiments, the engineered CRISPR-Tn systems are derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

In some embodiments, the system comprises two or more engineered CRISPR-To systems. Pairing of orthogonal systems with their orthogonal donor DNA substrates enables tandem insertion of multiple distinct payloads directly adjacent to each other without any risk of repressive effects from target immunity. For example, one, two, three, four, five, or more orthogonal CRISPR-Tn systems may be used to integrate large tandem arrays of payload DNA. In some embodiments, multiple orthogonal RNA-guided transposases and their transposon donor DNAs may be integrated into distal regions of a given chromosome or genome, such that the lack of sequence identity between the transposon ends of the distinct transposon DNA substrates prevents genetic instability and the risk of recombination.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell). Thus, in some embodiments, disclosed herein are systems or kits for DNA integration into a target nucleic acid sequence in a eukaryotic cell (e.g., a mammalian cell, a human cell).

a. CRISPR-Tn System

CRISPR-Cas systems are currently grouped into two classes (1-2), six types (I-VI) and dozens of subtypes, depending on the signature and accessory genes that accompany the CRISPR array. The engineered CRISPR-Tn system may be derived from a Class 1 CRISPR-Cas system or a Class 2 CRISPR-Cas system.

Type I CRISPR-Cas systems encode a multi-subunit protein-RNA complex called Cascade, which utilizes a crRNA (or guide RNA) to target double-stranded DNA during an immune response. Cascade itself has no nuclease activity, and degradation of targeted DNA is instead mediated by a trans-acting nuclease known as Cas3.

The present system may be derived from a Type I CRISPR-Cas system (such as subtypes I-B and I-F, including I-F variants. In some embodiments, the engineered CRISPR-Tn system is a Type I-F system. In some embodiments, the engineered CRISPR-Tn system is a Type I-F3 system.

In some embodiments, the engineered CRISPR-Tn system comprises Cas5, Cas6, Cas7, Cas8, or any combination thereof. In some embodiments, the engineered CRISPR-Tn system comprises Cas8-Cas5 fusion protein.

In certain embodiments, the Cas6 protein is encoded by a nucleic acid sequence having at least 70% similarity (e g., at least 70%, at least 75%, at least 80%, at least 85%, at least 90%, at least 95%, at least 98%, at least 99%) to that of SEQ ID NO: 14, SEQ ID NO: 30, SEQ ID NO: 46, or SEQ ID NO: 64. In certain embodiments, the Cas6 protein is encoded by the nucleic acid sequence of SEQ ID NO. 14, SEQ ID NO: 30, SEQ ID NO: 46, or SEQ ID NO: 64.

In certain embodiments, the Cas7 protein is encoded by a nucleic acid sequence having at least 70% similarity to that of SEQ ID NO: 12, SEQ ID NO: 28, SEQ ID NO: 44, or SEQ ID NO: 62. In certain embodiments, the Cas7 protein is encoded by a nucleic acid sequence of SEQ ID NO: 12, SEQ ID NO: 28, SEQ ID NO: 44, or SEQ ID NO: 62.

In certain embodiments, the Cas8-Cas5 fusion protein is encoded by a nucleic acid sequence having at least 70% similarity to that of SEQ ID NO: 10, SEQ ID NO: 26, SEQ ID NO: 42, or SEQ ID NO: 60. In certain embodiments, the Cas8-Cas5 fusion protein is encoded by a nucleic acid sequence of SEQ ID NO: 10, SEQ ID NO: 26, SEQ ID NO: 42, or SEQ ID NO: 60.

However, the invention is not limited to these exemplary sequences. Indeed, genetic sequences can vary between different strains, and this natural scope of allelic variation is included within the scope of the invention.

In certain embodiments, the Cas6 protein comprises an amino acid sequence having at least 70% similarity to that of SEQ ID NO: 13, SEQ ID NO: 29, SEQ ID NO: 45, or SEQ ID NO: 63. In certain embodiments, the Cas6 protein comprises the amino acid sequence of SEQ ID NO: 13, SEQ ID NO: 29, SEQ ID NO: 45, or SEQ ID NO: 63.

In certain embodiments, the Cas7 protein comprises an amino acid sequence having at least 70% similarity to that of SEQ ID NO: 11, SEQ ID NO: 27, SEQ ID NO: 43, or SEQ ID NO: 61. In certain embodiments, the Cas7 protein comprises the amino acid sequence of SEQ ID NO: 11, SEQ ID NO: 27, SEQ ID NO: 43, or SEQ ID NO: 61

In certain embodiments, the Cas8-Cas5 fusion protein comprises an amino acid sequence having at least 70% similarity to that of SEQ ID NO: 9, SEQ ID NO: 25, SEQ ID NO: 41, or SEQ ID NO: 59. In certain embodiments, the Cas8-Cas5 fusion protein comprises the amino acid sequence of SEQ ID NO: 9, SEQ ID NO: 25, SEQ ID NO: 41, or SEQ ID NO: 59.

A system of the present invention may comprise one or more transposon-associated proteins (e.g., transposases or other components of a transposon). The transposon-associated proteins may facilitate recognition or cleavage of the target nucleic acid and subsequent insertion of the donor nucleic acid into the target nucleic acid.

In some embodiments, the transposon-associated proteins are derived from a Tn7 or Tn7-like transposon. Tn7 and Tn7-like transposons may be categorized based on the presence of the hallmark DDE-like transposase gene, tnsB (also referred to as tniA), the presence of a gene encoding a protein within the AAA+ ATPase family, ms((also referred to as tniB), one or more targeting factors that define integration sites (which may include a protein within the tniQ) family, also referred to as tsD), but sometimes includes other distinct targeting factors), and inverted repeat transposon ends that typically comprise multiple binding sites thought to be specifically recognized by the TnsB transposase protein. In Tn7, the targeting factors, or “target selectors,” comprise the genes tnsD) and tnsE. Based on biochemical and genetics studies, it is known that TnsD binds a conserved attachment site in the 3′ end of the glmS gene, directing downstream integration, whereas TnsE binds the lagging strand replication fork and directs sequence-non-specific integration primarily into replicating/mobile plasmids.

The most well-studied member of this family of transposons is Tn7, hence why the broader family of transposons may be referred to as Tn7-like. “Tn7-like” term does not imply any particular evolutionary relationship between In7 and related transposons; in some cases, a Tn7-like transposon will be even more basal in the phylogenetic tree and thus Tn7 can be considered as having evolved from, or derived from, this related Tn7-like transposon.

Whereas Tn7 comprises tnsD) and msE target selectors, related transposons comprise other genes for targeting. For example, Tn5090/Tn5053 encode a member of the miQ family (a homolog of E. coli tnsD) as well as a resolvase gene miR; Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TiQ for RNA-guided mobilization; and other transposons encode Type V-US CRISPR-Cas systems that work together with TniQ for random and RNA-guided mobilization. Any of the above transposon systems are compatible with the systems and methods described herein.

In some embodiments, the one or more transposon-associated proteins comprise TnsA, TnsB, TnsC, or a combination thereof. In some embodiments, the one or more transposon-associated proteins comprise TnsB and TnsC. In some embodiments, the one or more transposon-associated proteins comprise TnsA, TnsB, and TasC.

In certain embodiments, the TnsA protein is encoded by a nucleic acid sequence having at least 70% similarity (e.g., at least 70%, at least 75%, at least 80%, at least 85%, at least 90%, at least 95%, at least 98%, at least 99%) to that of SEQ ID NO: 2, SEQ ID NO: 18, SEQ ID NO: 34, or SEQ ID NO: 50. In certain embodiments, the InsA protein is encoded by the nucleic acid sequence of SEQ ID NO: 2, SEQ ID NO: 18, SEQ ID NO: 34, or SEQ ID NO: 50.

In certain embodiments, the TnsB protein is encoded by a nucleic acid sequence having at least 70% similarity to that of SEQ ID NO. 4, SEQ ID NO. 20, SEQ ID NO: 36, or SEQ ID NO: 52. In certain embodiments, the TnsB protein is encoded by a nucleic acid sequence of SEQ ID NO: 4, SEQ ID NO: 20, SEQ ID NO: 36, or SEQ ID NO: 52.

In certain embodiments, the TnsC protein is encoded by a nucleic acid sequence having at least 70% similarity to that of SEQ ID NO: 6, SEQ ID NO: 22, SEQ ID NO: 38, or SEQ ID NO: 54. In certain embodiments, the TnsC protein is encoded by a nucleic acid sequence of SEQ ID NO: 6, SEQ ID NO: 22, SEQ ID NO: 38, or SEQ ID NO: 54.

However, the invention is not limited to these exemplary sequences. Indeed, genetic sequences can vary between different strains, and this natural scope of allelic variation is included within the scope of the invention.

In certain embodiments, the TnsA protein comprises an amino acid sequence having at least 70% similarity to that of SEQ ID NO: 1, SEQ ID NO: 17, SEQ ID NO: 33, or SEQ ID NO: 49. In certain embodiments, the TnsA protein comprises the amino acid sequence of SEQ ID NO: 1, SEQ ID NO: 17, SEQ ID NO: 33, or SEQ ID NO: 49.

In certain embodiments, the TnsB protein comprises an amino acid sequence having at least 70% similarity to that of SEQ ID NO: 3, SEQ ID NO: 19, SEQ ID NO: 35, or SEQ ID NO: 51. In certain embodiments, the TnsB protein comprises the amino acid sequence of SEQ ID NO: 3, SEQ ID NO: 19, SEQ ID NO: 35, or SEQ ID NO: 51.

In certain embodiments, the TnsC protein comprises an amino acid sequence having at least 70% similarity to that of SEQ ID NO: 5, SEQ ID NO: 21, SEQ ID NO: 37, or SEQ ID NO: 53. In certain embodiments, the TnsC protein comprises the amino acid sequence of SEQ ID NO: 5, SEQ ID NO: 21, SEQ ID NO: 37, or SEQ ID NO: 53.

In some embodiments, the at least one transposon protein comprises a TnsA-TosB fusion protein. TnsA and TnsB can be fused in any orientation: N-terminus to C-terminus; C-terminus to N-terminus; N-terminus to N-terminus; or C-terminus to C-terminus, respectively. Preferably the C-terminus of TnsA is fused to the N-terminus of TnsB.

In some embodiments, the TnsA-TnsB fusion may be fused using an amino acid linker peptide of various lengths to provide greater physical separation and allow more spatial mobility between the fused portions. The linker may comprise any amino acids and may be of any length. In some embodiments, the linker may be less than about 50 (e.g., 40, 30, 20, 10, or 5) amino acid residues.

In some embodiments, the linker is a flexible linker, such that InsA and TnsB can have orientation freedom in relationship to each other. For example, a flexible linker may include amino acids having relatively small side chains, and which may be hydrophilic. Without limitation, the flexible linker may contain a stretch of glycine and/or serine residues. In some embodiments, the linker comprises at least one glycine-rich region. For example, the glycine-rich region may comprise a sequence comprising [GS]n, wherein n is an integer between 1 and 10.

In some embodiments, the linker further comprises a nuclear localization sequence (NLS). The NLS may be embedded within a linker sequence, such that it is flanked by additional amino acids. In some embodiments, the NLS is flanked on each end by at least a portion of a flexible linker. In some embodiments, the NLS is flanked on each end by a glycine rich region of the linker. Suitable nuclear localization sequences for use with the disclosed system are described further below and are applicable to use with the TnsA-TnsB fusion protein. In some embodiments, the linker comprises the amino acid sequence of

(SEQ ID NO: 86)
GCGCGKRTADGSEFESPKKKRKVGSGSGG.

In certain embodiments, the TnsA-TnsB fusion protein comprises an amino acid sequence having at least 70% (at least 75%, at least 80%, at least 85%, at least 90%, at least 95%, at least 98%, at least 99%) similarity to that of SEQ ID NOs: 94-99. For example, the TnsA-TnsB fusion protein may comprise an amino acid sequence having one or more (e.g., 1, 2, 3, 4, 5, 6, 7, 8, 9, 10, 12, 15, 18, or 20) substitutions compared to that of SEQ ID NOs: 94-99. (0142| In some embodiments, the disclosed systems further comprise TnsD, TniQ, or a combination thereof or a nucleic acid encoding TnsD, TniQ, or a combination thereof. Thus, the one or more transposon-associated proteins may comprise TnsD, TniQ, or a combination thereof.

In certain embodiments, the TnsD protein is encoded by a nucleic acid sequence having at least 70% similarity to that of SEQ ID NO: 56. In certain embodiments, the TnsD protein is encoded by a nucleic acid sequence of SEQ ID NO. 56.

In certain embodiments, the TniQ protein is encoded by a nucleic acid sequence having at least 70% similarity to that of SEQ ID NO: 8, SEQ ID NO: 24, SEQ ID NO: 40, or SEQ ID NO: 58. In certain embodiments, the TniQ protein is encoded by a nucleic acid sequence of SEQ ID NO: 8, SEQ ID NO: 24, SEQ ID NO: 40, or SEQ ID NO: 58.

In certain embodiments, the TnsD protein comprises an amino acid sequence having at least 70% similarity to that of SEQ ID NO: 55. In certain embodiments, the TnsD protein comprises the amino acid sequence of SEQ ID NO: 55.

In certain embodiments, the TniQ protein comprises an amino acid sequence having at least 70% similarity to that of SEQ ID NO: 7, SEQ ID NO: 23, SEQ ID NO: 39, or SEQ ID NO: 57. In certain embodiments, the TniQ protein comprises the amino acid sequence of SEQ ID NO: 7, SEQ ID NO: 23, SEQ ID NO: 39, or SEQ ID NO: 57.

In some embodiments, the system comprises InsA, TnsB, TnsC, TnsD and TniQ. In some embodiments, the system comprises Cas5, Cas6, Cas7, Cas8, TnsA, InsB, TnsC, and at least one or both of TnsD or TniQ. In certain embodiments, the system comprises TnsD. In certain embodiments, the system comprises TniQ. In certain embodiments, the system comprises TnsD and TniQ.

In some embodiments, any combination of the at least one Cas protein and the at least one transposon associate protein may be expressed as a single fusion protein. In some embodiments, each of the at least one Cas protein and one or more of the at least one transposon-associated protein are part of a single fusion protein in which the components are expressed as a single megapeptide.

Sequences of exemplary Cas proteins, transposon-associated proteins, gRNAs, and transposon ends can also be found in International Patent Application WO2020181264, incorporated herein by reference. However, the invention is not limited to the disclosed or referenced exemplary sequences. Indeed, genetic sequences can vary between different strains, and this natural scope of allelic variation is included within the scope of the invention.

In other embodiments, any of the proteins described or referenced herein may comprise a sequence corresponding to, or substantially corresponding to, the wild-type version of the protein. For example, the sequence may substantially correspond to the wild-type protein sequence except for changes made for facile cloning or removal of known restriction sites. Thus, protein products from potential alternative start codons compared to the predicted nucleic acid sequences in this document are therefore not excluded.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The components of the system may be present in the system in various ratios. In some embodiments, each of the protein components or the nucleic acids encoding thereof are provided in a 1:1 ratio. For example, when each protein component is encoded on a single nucleic acid, the single nucleic acid comprises a single coding sequence for each protein component.

In some embodiments, any one of the protein components may be provided in greater abundance to any other protein component. In certain embodiments, Cas7 or the nucleic acid encoding Cas7 in greater abundance compared to the remaining protein components or nucleic acids encoding thereof. For example, multiple copies of a nucleic acid encoding Cas7 may be provided for each copy of any of the other components (e.g., Cas6, Cas5, Cas8, InsA, TnsB, or TnsC). In some embodiments, Cas7 is encoded on a nucleic acid separate from any of the other components such that it can be provided in the system and methods herein at a higher abundance or dosage than the other components. Analogously, higher concentrations of the Cas7 protein can be provided in the systems and methods compared to the other proteins. In some embodiments, for every one copy of Cas6 or Cas8, or nucleic acids encoding thereof, 2 or more copies of Cas7 or a nucleic acid encoding Cas7 are included in the system. In some embodiments, for every one copy of Cas6 or Cas8 or nucleic acids encoding thereof, 5-10 copies of Cas7 or a nucleic acid encoding Cas7 are included in the system.

b. Nuclear Localization Sequence

In the systems disclosed herein, one or more of the at least one Cas protein and the at least one transposon-associated protein comprise a nuclear localization signal (NLS). The nuclear localization sequence may be appended to the one or more of the at least one Cas protein and the at least one transposon-associated protein at a N-terminus, a C-terminus, embedded in the protein (e.g., inserted internally within the open reading frame (ORF)), or a combination thereof.

In some embodiments, one or more of the at least one Cas protein and the at least one transposon-associated protein comprises two or more NLSs. The two or more NLSs may be in tandem, separated by a linker, at either end terminus of the protein, or embedded in the protein (e.g., inserted internally within the ORF instead).

In some embodiments, a NLS is fused to the C-terminus of Cas6. In some embodiments, a NLS is fused to the N-terminus, C-terminus, or both of Cas7. In certain embodiments, Cas7 comprises two NLSs fused in tandem to the N-terminus. In some embodiments, a NLS is fused to the N-terminus or C-terminus of a Cas8-Cas5 fusion protein.

In some embodiments, a NLS is fused to the C-terminus of TnsA. In some embodiments, a NLS is fused to a N-terminus of TnsB. In some embodiments, a NLS is fused to the C-terminus of TnsC.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprise a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR[PAATKKAGQA]KKKK (SEQ ID NO: 87), and the NLS of EGL-13, MSRRRKANPTKLSENAKKLAKEVEN (SEQ ID NO: 88). In some embodiments, the NLS comprises a bipartite SV40 NLS. In certain embodiments, the NLS comprises an amino acid sequence having at least 70% similarity to KRTADGSEFESPKKKRKV(SEQ ID NO: 89). In select embodiments, the NLS consists of an amino acid sequence of KRTADGSEFESPKKKRKV(SEQ ID NO: 89).

The protein components of the disclosed system (e.g., the Cas proteins or the transposon-associated proteins) may further comprise an epitope tag (e.g., 3×FLAG tag, an HA tag, a Myc tag, and the like). In some embodiments, the epitope tag may be adjacent, either upstream or downstream, to a nuclear localization sequence. The epitope tags may be at the N-terminus, a C-terminus, or a combination thereof of the corresponding protein.

c. gRNA

In some embodiments, the engineered CRISPR-Tn systems further comprise a gRNA complementary to at least a portion of the target nucleic acid sequence, or a nucleic acid encoding the at least one gRNA.

The gRNA may be a crRNA, crRNA/tracrRNA (or single guide RNA, sgRNA). The terms “gRNA,” “guide RNA,” “crRNA,” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., the genome in a host cell). In some embodiments, the at least one gRNA is encoded in a CRISPR RNA (crRNA) array.

The system may further comprise a target nucleic acid. In some embodiments, target nucleic acid sequence comprises a human sequence.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLOS ONE, 10(3): (2015)); Zhu et al. (PLOS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096): 816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

As described elsewhere herein the protein and gRNA components of the system may be expressed and transcribed from the nucleic acids using any promoter or regulatory sequences known in the art. In some embodiments, the gRNA is transcribed under control of an RNA Polymerase II promoter. In some embodiments, the gRNA is transcribed under control of an RNA Polymerase III promoter.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The system may further comprise a target nucleic acid. The target nucleic acid may be flanked by a protospacer adjacent motif (PAM). A PAM site is a nucleotide sequence in proximity to a target sequence. For example, PAM may be a DNA sequence immediately following the DNA sequence targeted by the CRISPR-Tn system.

The target sequence may or may not be flanked by a protospacer adjacent motif (PAM) sequence. In certain embodiments, a nucleic acid-guided nuclease can only cleave a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, NGGNG and NNAGAAW (W=A or T, SEQ ID NO: 91), NNNNGATT (SEQ ID NO: 92), NAAR (R=A or G), NNGRR (R=A or G), NNAGAA (SEQ ID NO: 93) and NAAAAC (SEQ ID NO: 90), where N is any nucleotide. In some embodiments, the PAM may comprise a sequence of CN, in which N is any nucleotide. In select embodiments, the PAM may comprise a sequence of CC.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM. (0177| In some embodiments, when the system comprises TnsA, TnsB, TnsC, TnsD and TniQ binding to the target nucleic acid may be mediated through a TnsD binding site within the target nucleic acid sequence. Thus, the recognition of the target nucleic acid utilizing the systems described herein may proceed in a gRNA-dependent and/or -independent manner.

d. Donor Nucleic Acid

The system may further include a donor nucleic acid to be integrated. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like. In some embodiments, the donor nucleic acid comprises a cargo nucleic acid sequence.

The donor nucleic acid may be flanked by at least one transposon end sequence. In some embodiments, the donor nucleic acid is flanked on the 5′ and the 3′ end with a transposon end sequence. The term “transposon end sequence” refers to any nucleic acid comprising a sequence capable of forming a complex with the transposase enzymes thus designating the nucleic acid between the two ends for rearrangement. Usually, these sequences contain inverted repeats and may be about 10-150 base pairs long, however the exact sequence requirements differ for the specific transposase enzymes. Transposon end sequences are well known in the art. Transposon ends sequences may or may not include additional sequences that promotes or augment transposition.

The transposon end sequences on either end may be the same or different. The transposon end sequence may be the endogenous CRISPR-transposon end sequences or may include deletions, substitutions, or insertions. The endogenous CRISPR-transposon end sequences may be truncated. In some embodiments, the transposon end sequence includes an about 40 base pair (bp) deletion relative to the endogenous CRISPR-transposon end sequence. In some embodiments, the transposon end sequence includes an about 100 base pair deletion relative to the endogenous CRISPR-transposon end sequence. The deletion may be in the form of a truncation at the distal (in relation to the cargo) end of the transposon end sequences.

In some embodiments, the transposon end sequences may comprise a 250 bp nucleic acid sequence having at least 70% similarity to that of SEQ ID NO: 15, SEQ ID NO: 16, SEQ ID NO: 31, SEQ ID NO: 32, SEQ ID NO: 47, SEQ ID NO: 48, SEQ ID NO: 65, or SEQ ID NO: 66. In some embodiments, the sequences may contain a portion of the above disclosed sequences, thereby comprising a minimal end sequence for facilitation insertion.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or greater.

e. Nucleic Acids

The one or more nucleic acids encoding the engineered CRISPR-Tn system may be any nucleic acid including DNA, RNA, or combinations thereof. In some embodiments, the one or more nucleic acids comprise one or more messenger RNAs, one or more vectors, or any combination thereof.

The at least one Cas protein, the at least one transposon-associated protein (e.g., TnsA, TnsB, TnsC, TnsD, and TniQ), the at least one gRNA, and the donor nucleic acid may be on the same or different nucleic acids (e.g., vector(s)). In some embodiments, the at least one Cas protein and the at least one transposon associated protein (e.g., TnsA, TnsB, and TnsC) are encoded by different nucleic acids. In some embodiments, the at least one Cas protein and the at least one transposon associated protein (e.g., TnsA, TnsB, and TnsC) are encoded by a single nucleic acid. In some embodiments, the at least one gRNA is encoded by a nucleic acid different from the nucleic acid(s) encoding the at least one Cas protein and at least one transposon associated protein (e.g., TnsA, TnsB, and TnsC) In some embodiments, the at least one gRNA is encoded by a nucleic acid also encoding the at least one Cas protein, at least one transposon associated protein (e.g., TnsA, TnsB, and TnsC), or both. In some embodiments, the nucleic acid encoding the at least one Cas protein, at least one transposon associated protein (e.g., TnsA, TnsB, and TnsC), the at least one gRNA, or any combination thereof further comprises the donor nucleic acid.

In select embodiments, a single nucleic acid encodes the gRNA and at least one Cas protein. For example, in certain embodiments, a single nucleic acid encodes the gRNA and Cas6. In alternative embodiments, a single nucleic acid encodes the gRNA and Cas7.

The gRNA may be encoded anywhere in the nucleic acid encoding the at least one Cas protein. In some embodiments, the gRNA is encoded in the 3′ UTR of the Cas protein-coding gene.

The one or more nucleic acids encoding the protein components may further comprise, in the case of RNA, or encode, as in the case of DNA, a sequence capable of forming a triple helix adjacent to the sequence encoding the protein component. In some embodiments, the sequence capable of forming a triple helix is downstream of the sequence encoding the at least one Cas protein and/or the sequence encoding the at least one transposon-associated protein. In some embodiments, the sequence capable of forming a triple helix is in a 3′ untranslated region of the sequence encoding the at least one Cas protein or the sequence encoding the at least one transposon-associated protein.

A tiple helix is formed after the binding of a third strand to the major groove of a duplex nucleic acid through Hoogsteen base pairing (e.g., hydrogen bonds) while maintaining the duplex structure of two strands making the major groove. Pyrimidine-rich and purine-rich sequences (e.g., two pyrimidine tracts and one purine tract or vice versa) can form stable triplex structures as a consequence of the formation of triplets (e.g., A-U-A and C-G-C).

In some embodiments, the triple helix forming sequence comprises two uracil-rich tracts and an adenosine-rich tract, each separated by linker or loop regions. As used herein, the term “A-rich tract” refers to a strand of consecutive nucleosides in which at least 80% of the consecutive nucleosides are adenosine. Similarly, the term “U-rich motif” refers to a strand of consecutive nucleosides in which at least 80% of the consecutive nucleosides are uridine.

In some embodiments, the triple helix sequence is derived from the 3′ terminal triple helix sequences of triple helix terminators from a long non-coding RNAs (lncRNAs), e.g., metastasis-associated lung adenocarcinoma transcript 1 (MALAT1).

One or more of the at least one Cas protein and the at least one transposon-associated protein comprise a sequence of an internal ribosome entry site (IRES) or a ribosome skipping peptide. This is particularly advantageous when a single nucleic acid or vector is used to express multiple components of the system.

The ribosome skipping peptide may comprise a 2A family peptide 2A peptides are short (˜18-25 aa) peptides derived from viruses. There are four commonly used 2A peptides, P2A, T2A, E2A and F2A, that are derived from four different viruses. Any known 2A peptide sequence is suitable for use in the disclosed system.

In some embodiments, the nucleic acid encoding the at least one Cas protein, the at least one transposon-associated protein, the at least one gRNA, or any combination thereof further comprises the donor nucleic acid.

In certain embodiments, engineering the system for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons. Furthermore, in some embodiments, engineering the CRISPR-Cas system involves incorporating elements of the native CRISPR array into the disclosed system.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example, this may allow for DNA integration by transforming bacteria of interest, but then being left with engineered strains that have no memory of the plasmids or vectors used for the integration.

Drug selection strategies may be adopted for positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the present system (such as one or more Cas proteins and/or Tns proteins, gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1): 33-40; and Walther W. and Stein U., 2000 Drugs, 60(2): 249-71, incorporated herein by reference.

In one embodiment, a DNA segment encoding the present protein(s) is contained in a plasmid vector that allows expression of the protein(s) and subsequent isolation and purification of the protein produced by the recombinant vector. Accordingly, the proteins disclosed herein can be purified following expression, obtained by chemical synthesis, or obtained by recombinant methods.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N. Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EFla (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBb (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α.) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′-and 3′-untranslated regions for mRNA stability and translation efficiency from highly-expressed genes like α-globin or β-globin; SV40 polyoma origins of replication and Co1E1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRPI genes of S. cerevisiae.

When introduced into the cell, the vectors may be maintained as an autonomously replicating sequence or extrachromosomal element or may be integrated into host DNA.

In one embodiment, the donor DNA may be delivered using the same gene transfer system as used to deliver the Cas protein, and/or transposon associated proteins (included on the same vector) or may be delivered using a different delivery system In another embodiment, the donor DNA may be delivered using the same transfer system as used to deliver gRNA(s).

In one embodiment, the present disclosure comprises integration of exogenous DNA into the endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, donor polynucleotides and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110(6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

Exemplary vectors encoding the systems described herein are provided in SEQ ID NOs: 67-78 and 100-292.

### Methods

Also disclosed herein are methods for nucleic acid integration utilizing the disclosed systems or kits. The methods may comprise contacting a target nucleic acid sequence with a system disclosed herein or a composition comprising the system. The descriptions and embodiments provided above for the engineered CRISPR-Tn system, the gRNA, and the donor nucleic acid are applicable to the methods described herein.

The target nucleic acid sequence may be in a cell. In some embodiments, the contacting a target nucleic acid sequence comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, IRNA, micro RNA (miRNA), and small interfering RNA (siRNA), and coding RNA, such as messenger RNA (mRNA). In some embodiments, the target nucleic acid sequence encodes a protein or polypeptide.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

The method may comprise administering to the subject, in vivo, or by transplantation of ex vivo treated cells, an effective amount of the described system. In some embodiments, the vector(s) is delivered to the tissue of interest by, for example, an intramuscular, intravenous, transdermal, intranasal, oral, mucosal, or other delivery methods.

The components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA integration is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine, preservatives; low molecular weight polypeptides, proteins, such as serum albumin, gelatin, or immunoglobulins; amino acids; hydrophobic polymers; monosaccharides; disaccharides, and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The methods may be used for a variety of purposes. For example, the methods may include, but are not limited to, inactivation of a microbial gene, RNA-guided DNA integration in a plant or animal cell, methods of treating a subject suffering from a disease or disorder (e.g., cancer, Duchenne muscular dystrophy (DMD), sickle cell disease (SCD), β-thalassemia, and hereditary tyrosinemia type I (HT1)), and methods of treating a diseased cell (e.g., a cell deficient in a gene which causes cancer).

### Kits

Also within the scope of the present disclosure are kits that include the components of the present system.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The present disclosure also provides for kits for performing DNA integration in vitro. The kit may include the components of the present system. Optional components of the kit include one or more of the following: buffer constituents, control plasmid, sequencing primers, cells.

### EXAMPLES

The following are examples of the present invention and are not to be construed as limiting.

### Materials and Methods

Type 1-F3 CRISPR-Tn detection Protein sequences corresponding to Vibrio cholerae TnsA, TnsB, TnsC, TniQ, Cas8, Cas7, and Cas6 from the Tn6677 transposon were used as queries for PSI-BLAST (ncbi-blast-2.10.0+ release) against the nr database (version 3/27/20) using the parameters: -evalue 0.005-num_alignments 9999999-num_iterations. Unique protein IDs were extracted from each PSI-BLAST result file and used for further analysis. The genomic accession ID corresponding to each protein ID was retrieved using NCBI Efetch, and genomic IDs with hits for TniQ, Cas8, Cas7, Cas6, TnsA, TnsB, and TnsC, referred to as the Minimal Gene Set (MGS), formed an initial set of potential homologs. A genomic accession ID was scored as containing a type I-F CRISPR-Tn system if it contained PSI-BLAST hits in the following order (with no restriction on the linear distance between each PSI-BLAST hit):
1) [TnsA, TnsB, TnsC, TniQ,Cas8,Cas7,Cas6]
2) [TnsA, TnsB, TnsC, Cas6, Cas7, Cas8, TniQ]
3) [TnsC,TnsB, TnsA, Cas6, Cas7,Cas8, TniQ]
4) [Cas6, Cas7,Cas8, TniQ, TnsC, TnsB, TnsA]
5) [TniQ,Cas8, Cas7,Cas6, TnsC, TnsB, TnsA]
6) [Cas6, Cas7,Cas8, TniQ, TnsA, TnsB, TnsC]
7) [TniQ,Cas8, Cas7,Cas6, TnsA, TnsB, TnsC]
8) [TnsB, TnsA, TnsC, TniQ),Cas8, Cas7,Cas6]
9) [Cas6, Cas7,Cas8, TniQ, TnsC, TnsA, TnsB]
10) [TnsA, TnsB, TnsB, TnsC, TniQ,Cas8,Cas7,Cas6] (putative TnsB duplication)
11) [Cas6,Cas7,Cas8, TniQ,TnsC,TnsB, TnsB, TnsA] (putative TnsB duplication).

Transposon end prediction To determine the transposon ends of potential homolog systems, a user-defined length of genomic sequence (default=100000) upstream and downstream of the MGS was extracted using Entrez Programming Utilities. Genomic “flanks” upstream and downstream of the MGS were then used for target site duplication (TSD)+terminal inverted repeat (TIR) detection in intergenic regions. All open reading frames (ORFs) within the genomic flanks were predicted using EMBOSS getorf (minsize=200; table=11). All genomic sequences within predicted ORFs were excluded from the TSD+TIR search. A 5′ sliding window searched between the ORFs downstream of the transposon MGS for a 5 bp TSD candidate. For every TSD candidate, a 3′ sliding window searched upstream of the transposon MGS for a matching TSD candidate. Once a pair of 5′ and 3′ TSDs was found, the 3 bps upstream and downstream of the respective repeats were checked to match a TG/AC dinucleotide motif and complementarity.

To predict TnsB binding sites within putative transposon ends, a sliding window of length 18 bp was defined downstream of a putative 5′ TSD. In order to determine repeats on the same end, a second window iterated from the first window position until the 5′ MGS coordinated (or up to 500 bp). After each iteration, the hamming distance (defined as the number of mismatches) was calculated between the first and second windows. A match was registered if the sequences had Hamming distance <=3. All positions of the second sliding window that produce matches were recorded, along with the position of the first window. Subsequently, a third sliding window iterated from the 3′ TSD until the 3′ MGS coordinate (or up to 500 bp). The first sliding window was compared to the reverse complement of the third sliding window and registered a match if the sequences had Hamming distance <=3. The reverse complement was taken because TnsB binding sites in each transposon end were oriented in opposite directions. All positions of the third sliding window that produced matches were recorded, along with the position of the first window.

The above sliding window analysis yielded the hamming distance between all possible pairs of 18-mers, 500 bp from each transposon end. These data can be represented as a hamming distance matrix. Elements in this matrix can be plotted as a series of peaks, where the x-axis represents the distance from each transposon end, and the y-axis represents the number of matches between a window at particular position and all other windows, 500 bp from each transposon end. Matches that were very close to one another were clustered (for example, if two called peaks lie 1 bp from each other, they were merged). This clustered series of peaks represented TnsB binding site positions, relative to each transposon end. The corresponding 18 bp DNA sequences were retrieved and aligned using Clustal 1.2.4. In addition, 5 bp of flanking genomic DNA sequence was added to each aligned TnsB binding site to better visualize matching bases. The alignment was then piped into MView 1.65 to generate a consensus sequence.

Manual inspection and selection of type 1-F3 CRISPR-Tn CRISPR arrays were predicted using CRISPRCasFinder 4.2.2 (Standard settings: no Cas gene detection) and were checked for the presence of a CUGCC-like stem-loop in CRISPR repeats. Conservation of active site residues in TnsA, TnsB, TnsC, TniQ, and Cas6 were checked manually.

Experimental pipeline for type I-F3 CRISPR-Tn characterization Expression vectors (pEffector) were designed where a single T7 promoter drives the expression of a CRISPR array (repeat-spacer-repeat), the native tniQ-cas8-cas7-cas6 operon, and the native tnsA-tnsB-tnsC operon from a pCDF-Duet-1 backbone. The accompanying pDonor vectors were designed to encode 250 bp Left and Right transposon end sequences on either end of a chloramphenicol resistance gene, generating a mini-Tn of 1307-bp in size, on a pUC19 backbone. Single-plasmid vectors were designed by combining the mini-Tn and the protein-RNA expression cassette onto a single plasmid.

Table 1 contains a list of CRISPR-transposon systems and includes a In ID number, a simplified name of the system based on the species from which it derives, the entire species/strain information, and an NCBI genomic accession ID that encodes the transposon.

TABLE 1
Type I-F3 CRISPR-transposons and associated
name, species, and genomic ID

| | Simple | | Genomic |
|---|---|---|---|
| Tn ID | name | Species of origin | accession ID |
| Tn7003 | Vpa | Vibrio parahaemolyticus | CP023486.1 |
| | | FORC_071 | |
| Tn7008 | Asp | Aliivibrio sp. 18157 | MAJS01000006 |
| Tn7016 | PS983 | Pseudoalteromonas sp. S983 | PNDL01000005.1 |
| Tn7017 | Eas | Endozoicomonas ascidiicola | LUTV01000003.1 |
| | | strainAVMART05 | |

Names and sequences of pDonor plasmids are described in SEQ ID NOs: 67-70. Names and sequences of pEffector plasmid are described in SEQ ID NOs: 71-74. Names and sequences of pSPIN plasmids are described in SEQ ID NOs: 75-78.

CRISPR arrays were cloned as repeat-spacer-repeat arrays and are denoted “typical” for arrays containing canonical repeats from the primary CRISPR array derived from each transposon, or “atypical” for arrays that contain atypical repeats derived from the secondary CRISPR array that encodes homing site crRNAs. Representative typical and atypical CRISPR arrays for each CRISPR-Tn system are given in Table 2, using the spacer sequence for crRNA-4, as described previously (Klompe et al., 2019, Nature 571, 219-225, incorporated herein by reference).

TABLE 2
Sequence of typical and atypical CRISPR, as repeat-spacer-repeat array

| Ta ID | Typical CRISPR array | Atypical CRISPR Array |
|---|---|---|
| Tn7003 | GTGAACTGCCGAATAGGTAGCT | TCATTACTACTAAAAAGTAGCTGA |
| | GATAATAGTACAGCGCGGCTGA | TAACAGTACAGCGCGGCTGAAATC |
| | AATCATCATTAAAGCGGTGAAC | ATCATTAAAGCGGAATACTGCCGA |
| | TGCCGAATAGGTAGCTGATAAT | ACAGGTAGGAGGCTCA (SEQ ID |
| | (SEQ ID NO: 79) | NO: 83) |
| Tn7008 | GTAACCTGCCGGATAGGCAGCC | GTAACCTGCCGGATAGGCAGCCAA |
| | AAGAATAGTACAGCGCGGCTGA | GAATAGTACAGCGCGGCTGAAATC |
| | AATCATCATTAAAGCGGTAACC | ATCATTAAAGCGCTATTATGCTGG |
| | TGCCGGATAGGCAGCCAAGAAT | AAAAGCAGTAAAACAT (SEQ ID |
| | (SEQ ID NO: 80) | NO: 84) |
| Tn7016 | GTGACCTGCCGTATAGGCAGCT | GTGACCTGCCGTATAGGCAGCTGA |
| | GAAAATAGTACAGCGCGGCTGA | AGATAGTACAGCGCGGCTGAAATC |
| | AATCATCATTAAAGCGGTGACC | ATCATTAAAGCGTAATTCTGCCGA |
| | TGCCGTATAGGCAGCTGAAAAT | AAAGGCAGTGAGTAGT (SEQ ID |
| | (SEQ ID NO: 81) | NO: 85) |
| Tn7017 | CCTCACTGCCGCATACGCAGCT | |
| | GAAAATAGTACAGCGCGGCTGA | |
| | AATCATCATTAAAGCGCCTCACT | |
| | GCCGCATACGCAGCTGAAAAT | |
| | (SEQ ID NO: 82) | |

Transposition assays All transposition experiments were performed in E. coli BL21(DE3) cells (NEB). For experiments including pDonor and pEffector, chemically competent cells carrying one of the plasmids were prepared and, after transformation of the other plasmid, transformants were isolated by selective plating on double antibiotic LB-agar plates containing IPTG. For experiments with pSPIN vectors, transformants were plated on LB-agar plates containing spectinomycin and IPTG. Transformations were done through heat shock at 42° C. for 30 see, and after recovering cells in fresh LB medium at 37° C. for 1 h, cells were plated on LB-agar plates containing the appropriate antibiotics and inducer (100 μg ml⁻¹ carbenicillin, 50 μg ml⁻¹ spectinomycin, 0.1 mM IPTG). After overnight growth at 37° C. for 18 h, hundreds of colonies were scraped from the plates, resuspended in LB medium, and prepared for subsequent analysis. Experiments performed at 25° C. were incubated for 62 h instead. Cell lysates were then prepared as described previously (Klompe et al. (2019) Nature 577, 219-225, incorporated herein by reference). Tn7017 did not yield any colonies at 37° C.; lower incubation temperature may also be affecting integration efficiency through mitigating toxicity issues. Thirty-two base pair spacer sequences were used regardless of the length of the predicted natural att-spacer.

qPCR assay to determine transposition efficiency Pairs of transposon- and target DNA-specific primers were designed to amplify fragments resulting from RNA-guided DNA integration at the expected loci in either orientation. A separate pair of genome-specific primers was designed to amplify an E. coli reference gene (rssA) for normalization purposes. qPCR reactions (10 μl) contained 5 μl of SsoAdvanced Universal SYBR Green Supermix (BioRad), 1 μl H2O, 2 μl of 2.5 μM primers, and 2 μl of tenfold diluted lysate prepared from scraped colonies, as described for the PCR analysis above. Reactions were prepared in 384-well clear/white PCR plates (BioRad), and measurements were performed on a CFX384 Real-Time PCR Detection System (BioRad) using the following thermal cycling parameters: polymerase activation and DNA denaturation (98° C. for 2.5 min), 40 cycles of amplification (98° C. for 10 s, 62° C. for 20 s), and terminal melt-curve analysis (65-95° C. in 0.5° C. per 5 s increments). Each biological sample was analyzed in three parallel reactions: one reaction contained a primer pair for the E. coli reference gene, a second reaction contained a primer pair for one of the two possible integration orientations, and a third reaction contained a primer pair for the other possible integration orientation. Transposition efficiency for each orientation was calculated as 2ΔCq, in which ΔCq is the Cq difference between the experimental reaction and the control reaction. Total transposition efficiency for a given experiment was calculated as the sum of transposition efficiencies for both orientations. All measurements presented in the text and figures were determined from three independent biological replicates.

Methods of next-generation sequencing (NGS) to profile PAM and other libraries PCR products were generated with Q5 Hot Start High-Fidelity DNA Polymerase (NEB) from extracted genomic DNA (as described by the Wizard® Genomic DNA Purification Kit), miniprepped plasmid samples, or 20-fold diluted PCR1 samples. Reactions contained 200 μM dNTPs and 0.5 μM primers and were generally subjected to 20 or 10 thermal cycles (PCR1 and PCR2, respectively) with an annealing temperature of 65° C. Primer pairs contained one target-specific primer and one transposon-specific primer (output library), two pTarget-specific primers (PAM input library), or one pDonor backbone-specific primer and one transposon-specific primer (pDonor input library). PCR amplicons were resolved by 1-2% agarose gel electrophoresis and visualized by staining with SYBR Safe (Thermo Scientific), DNA was isolated by Gel Extraction Kit (Qiagen), and NGS libraries were quantified by qPCR using the NEBNext Library Quant Kit (NEB). Illumina sequencing was performed using a NextSeq mid or high output kit with 150-cycle reads and automated demultiplexing and adaptor trimming (Illumina).

PAM library experiments To determine the PAM preference for RNA-guided DNA-integration, the following steps were performed using custom Python scripts. First, reads were filtered based on the requirement that they contain 10 bp of perfectly matching transposon end sequence (in the case of the output library) as well as a perfect 32 bp target site. The five bases immediately upstream of the target site were then extracted, and enrichment values were calculated as:

((reads PAM output)/(total output reads))/((reads PAM input)/(total input reads)).

To determine the integration site preference from the same PAM library dataset reads were extracted from the output library that resulted from a ‘CC’ PAM sequence. These reads were then subjected to the illumina pipeline script as previously described (Vo et al., Nat Biotechnol 39, 480-489 (2021), incorporated herein by reference) that extracts a 17-bp fingerprint from the integration site, maps it back to the targeted sequence, and outputs plots of number of reads found per base position relative to the 3′ end of the target site.

pDonor library experiments A pDonor library encoding twenty different mini-Tn was generated and prepared for NGS as described above 1.5 μl of pDonor library was transformed with chemically competent E. coli BL21(DE3) cells containing a pEffector and plated on LB agar containing 100 μg ml⁻¹ carbenicillin, 50 μg ml⁻¹ spectinomycin, and 0.1 mM IPTG. After 18 hours, cells were scraped and resuspended in 500 μl of LB. An equivalent of 500 μl of OD7.0 was aliquoted for each sample and the gDNA was purified using a Promega Wizard Genomic DNA purification kit and used for NGS sample preparation as described above. Primer pairs contained one genome-specific primer and one cargo-specific primer and were varied such that both tRL and tLR integration orientations could be detected downstream of the target site.

Reads from the output libraries (the amplicons that result from integration at target-4) were filtered based on a perfect 20 bp sequence match to the target locus, and the presence of specific 15-bp mini-Tn ends was tallied. This was done for tRL integration only, but for both the left- and right-end boundaries. Reads for the input libraries (the amplicons resulting from the pDonor pooled library) were filtered based on a 45 bp sequence (25 bp transposon-end+20 bp flanking sequence) or 25 bp sequence (20 bp flanking+5 bp TSD) for the left- and right-end amplicons respectively, and the number of occurrences for each mini-Tn homolog were tallied. Enrichment values were then calculated as:

((reads mini-Tn output)/(total output reads))/((reads mini-Tn input)/(total input reads)).

Sequence and Phylogenetic Analyses CRISPR-Tn systems were clustered based on TnsB phylogeny as follows. Bioinformatic analysis resulted in 304 unique TnsB protein IDs that were found in genomic sequences together with all other required CRISPR-Tn protein components. This set was filtered for <90% sequence identity using CD-HIT with default settings. To generate a known outgroup for phylogenetic analysis, BLASTp was run with EcoTnsB (from Tn7) as a query, and 5 homologous sequences were extracted (HAW0448631.1, WP_000267723.1, EGT3574482.1, WP_126892736.1, and WP_087529690.1). TnsB protein sequences were then aligned in geneious using the MUSCLE plugin (default settings and allowing for 10 iterations), and the resulting alignment was used to generate a phylogenetic tree using the FastTree plugin (default settings). The EcoTnsB-derived sequences indeed formed a distinct clade and were used to root the tree, which was done using iTOL for downstream visualization purposes. Nodes with a bootstrap value <0.7 were removed, and clades were colored based on a branch length of 1.23.

Phylogenetic analyses of TniQ psiBLAST results were performed as follows. Protein sequences corresponding to TnsD/TniQ from Tn7, Tn6677, and Tn7017 (WP_001243518.1, WP_000479715.1, and WP_067516660.1+WP_157673483.1, respectively) were used as queries for PSI-BLAST (ncbi-blast-2.10.0+release) against the nr database (version 02/04/2021) using the parameters: -evalue 0.005-num_alignments 9999999-num_iterations 10. Unique protein IDs were extracted, combined, and filtered for <90% sequence identity using CD-HIT with default settings and protein lengths were plotted. To reduce the number of protein sequences for downstream analysis unique protein IDs were extracted, combined, and filtered for <50% sequence identity using CD-HIT with default settings. Because of the large number of homologs, and the large spread in protein sizes, only sequences 370-675 AA in size were included in downstream analysis. This list of 3,585 sequences was complemented with TnsD sequences identified in different studies: I-B1-TnsD (AvCAST-TnsD, WP_011320212.1); I-B2-TnsD (PmcCAST-TnsD, WP_094348672.1), 1-F3-TnsD (RLV60497.1, WP_170308330.1), and additional TnsD sequences from Tn7 to create an outgroup. The first 180 AA were extracted to solely compare the TniQ (pfam xxx) domain. Protein sequences were aligned in geneious using the MUSCLE plugin (default settings, 2 iterations), from which a phylogenetic tree was generated using the FastTree plugin (default settings).

Smaller scale analysis was performed with selected TnsD/TniQ protein sequences: twenty type I-F3, two type I-B, and three type V-K CRISPR-Tn. Additionally, two predicted type 1-F3 systems and the flagship Tn7-TnsD were included. Sequences were aligned in geneious using the MUSCLE algorithm with default settings and allowing for 8 iterations. The sequence identity matrix was exported and visualized in Prism. FastTree was then used with default settings to generate a phylogenetic tree, which was uploaded to iTOL for visualization purposes. The three type V-K systems were used as an outgroup to root the tree.

Cargo analyses of CRISPR-Tn systems were performed as follows. Pfam identifiers were assigned for annotated genes within each full length transposon, and manually compared to lists of pfams predicted to be associated with bacterial defense systems.

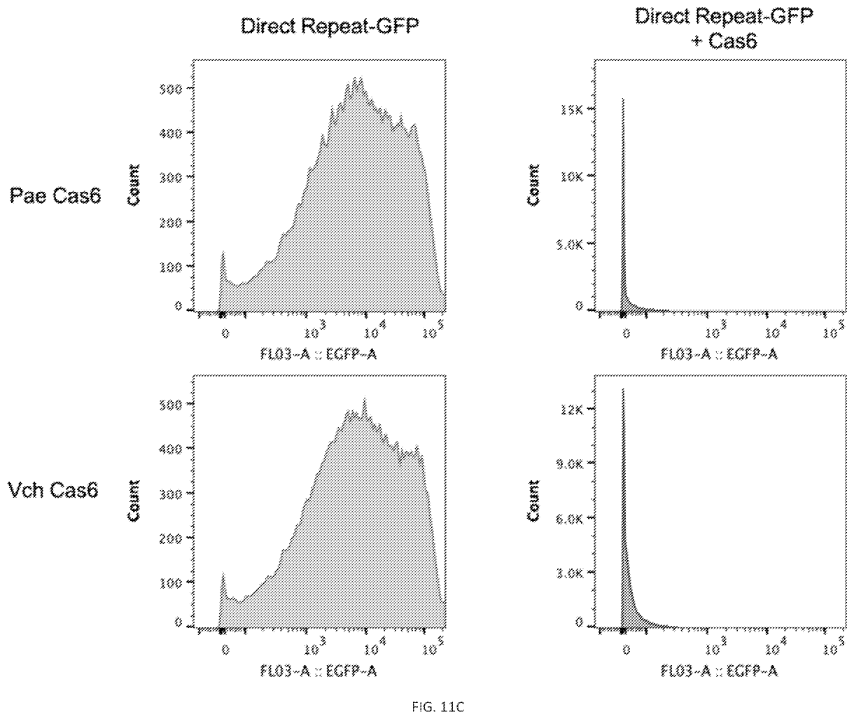

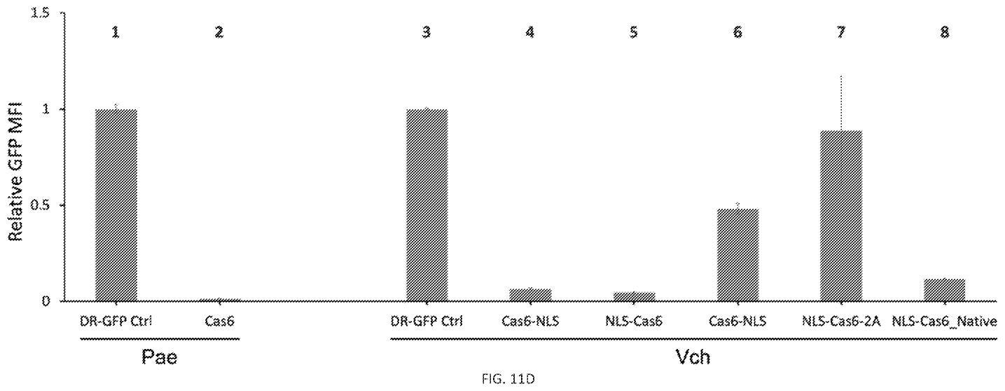

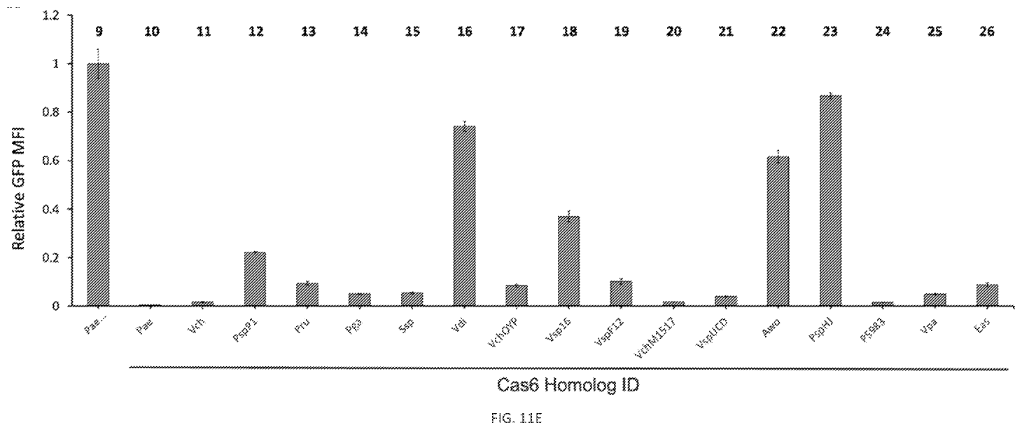

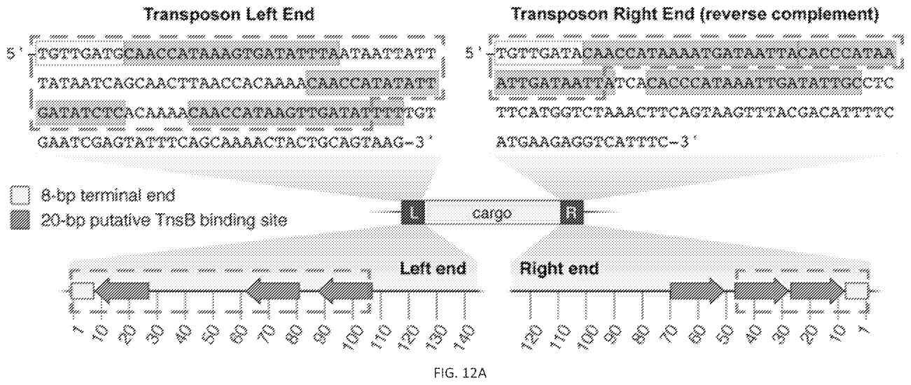

Experimental results presented herein and described in accompanying figures employed a large set of variable gRNA and protein expression vectors, as well as, in some cases, donor DNA and target DNA vectors. Results presented in bar graphs and elsewhere are accompanied by an experimental numeric ID (see FIG. 11, for an example), which is linked with information provided in Table 3, for Examples 5-11. This table provides a key describing the vectors (aka plasmids) that were used, for the same experimental numeric ID. Descriptions of the Plasmids usedin Examples 5-11 are in Tables 4-7. Results presented in Example 12 are accompanied by an experimental numeric ID, which is linked with information provided in Table 8 and descriptions of the Plasmids are in Table 9. Results presented in Example 13 are linked with information provided in Table 10.

### Example 1

Identification and characterization of active Type I-F3 CRISPR-Tn systems

To explore the natural mechanistic variance among CRISPR-Tn, a bioinformatic pipeline was established to identify and prioritize Type I-F3 CRISPR-Tn systems for experimental analysis. Briefly, V. cholerae protein components from Tn6677 were used as a query and iterative rounds of psiBLAST were performed to assemble homolog sets, genomic contigs encoding all protein components were extracted, and left and right transposon boundaries were identified based on their characteristic structure. Enzymatic active sites and CRISPR arrays were manually inspected for a subset of candidate systems, and systems from a range of gammaproteobacterial species whose TnsB transposase proteins are well distributed across a number of clearly distinguishable clades were selected. Species and naming information for each CRISPR-Tn are given in Table 1.

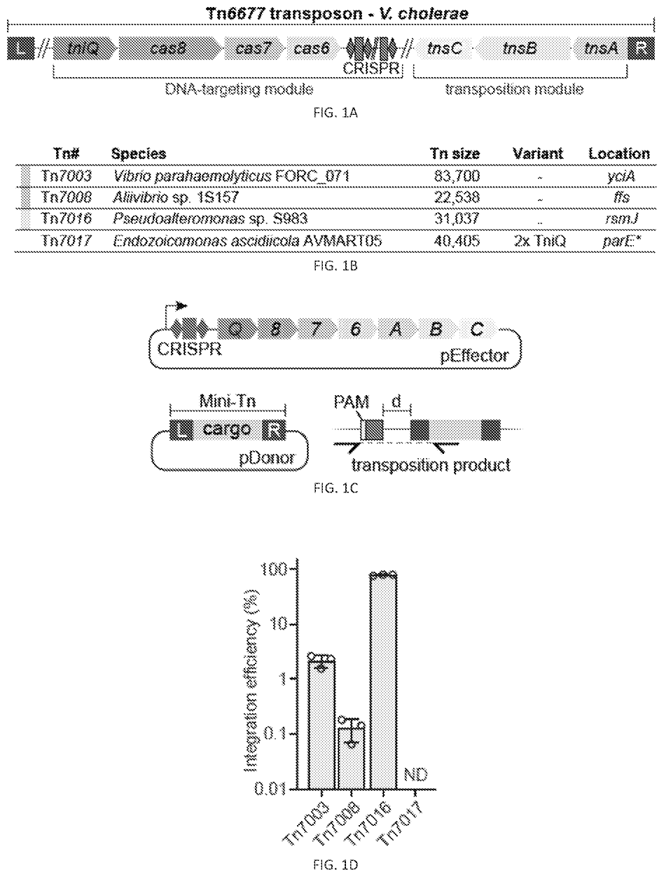

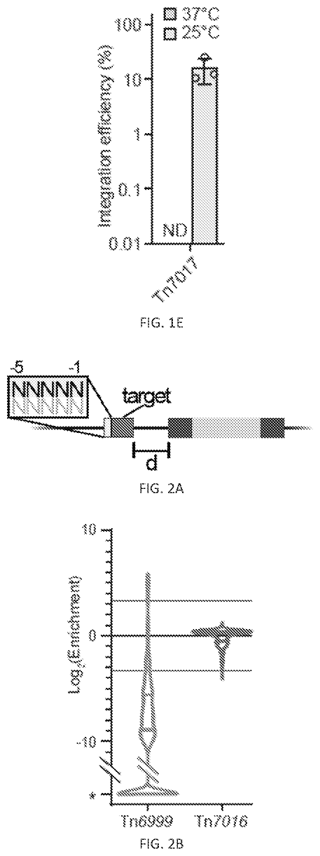

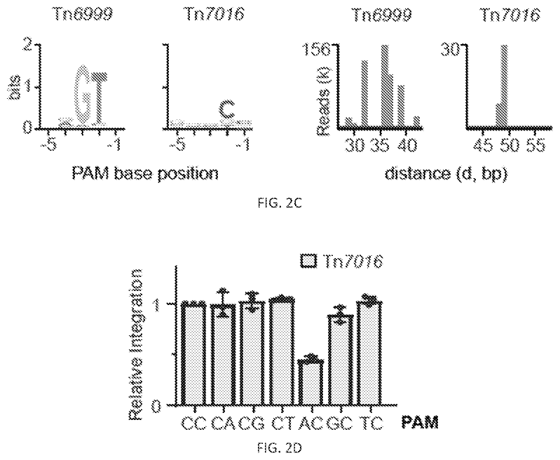

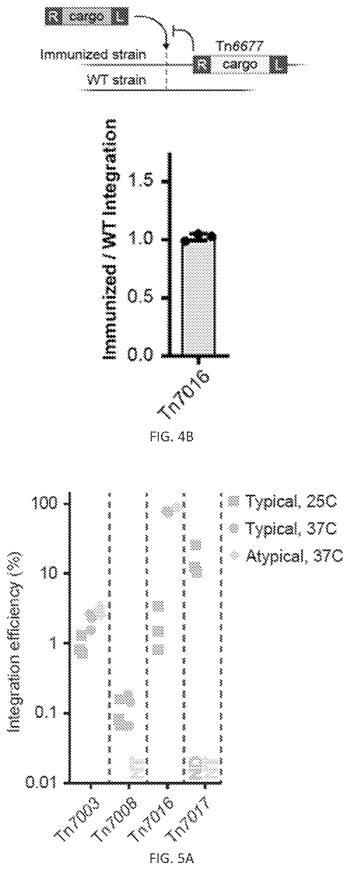

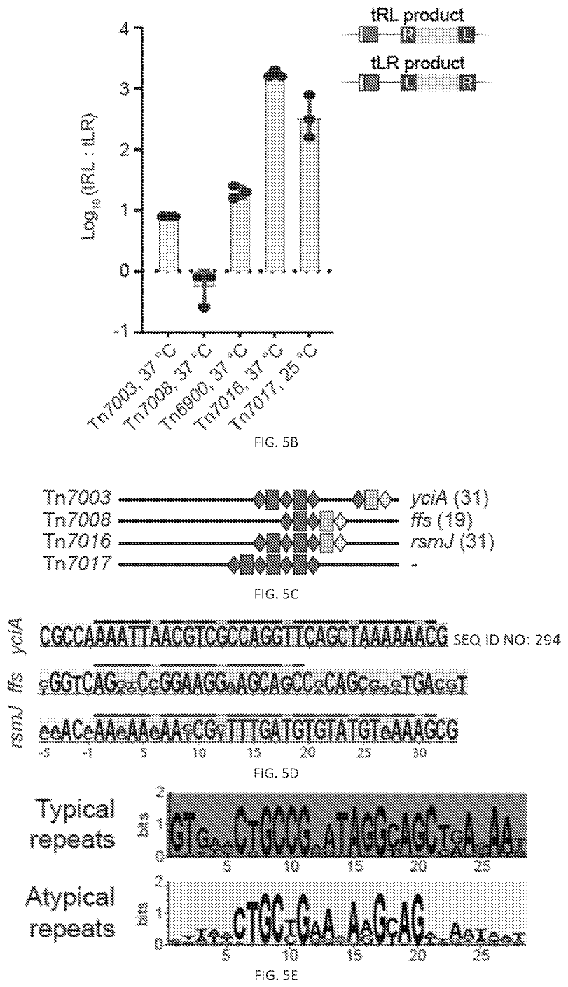

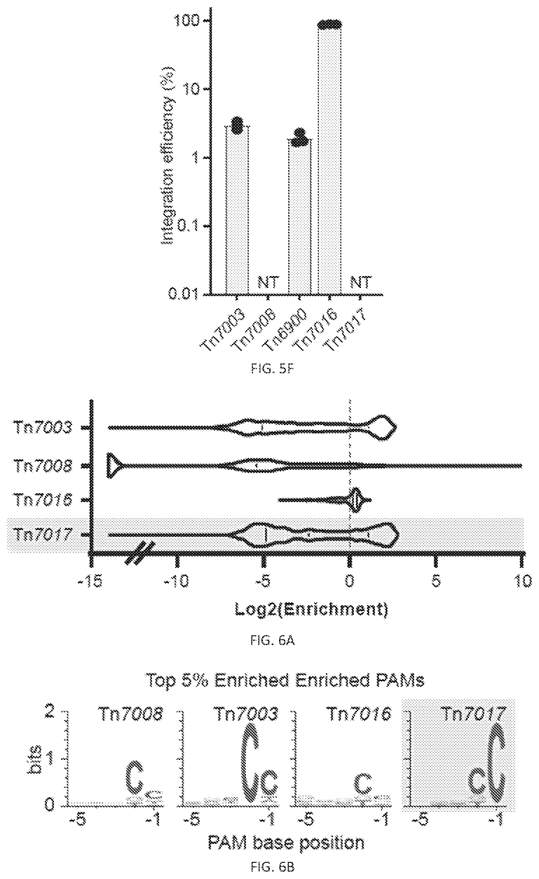

For each system, a donor plasmid (pDonor) was synthesized and cloned encoding the mini-Tn, alongside an effector plasmid (pEffector) that encodes a crRNA and 6-8 protein components. Sequences of these plasmids are given in SEQ ID NO: 67-74. Transposition was assayed in E. coli BL21(DE3) cells using a crRNA targeting lacZ, and integration events in either of two possible orientations were quantified using qPCR (FIG. 1C). The majority of systems were functional at 37° C., albeit with a range of activities, with one catalyzing targeted integration at near 100% efficiency without selection for the insertion event (FIG. 1D). Since many systems derive from species that grow at lower temperatures, the transposition assays were repeated at 25° C. and activity was greatly improved for Tn7017 (FIGS. 1E and 5A). Bidirectional integration was analyzed, finding that most favored one orientation product, with some showing a >10³: 1 preference (FIG. 5B).

In addition to their standard CRISPR arrays, both I-F3 and V-K CRISPR-Tn systems encode atypical CRISPR RNAs that direct homing to specific genomic attachment sites and are characterized by unusual repeats and spacers. In some cases, these atypical crRNAs are differentially regulated, or direct enhanced integration activity when compared to typical crRNAs. The atypical CRISPR arrays for each of the disclosed systems were tested for integration efficiency at the same target site using these atypical repeats with fully matching spacer sequences (FIGS. 5C-5F). Sequences for representative typical and atypical CRISPR arrays were each system, with a crRNA-4 spacer sequence, are given in Table 2.

### Example 2

RNA-guided transposition with I-F3 systems exhibits flexible PAM requirements

Canonical DNA-targeting CRISPR-Cas systems rely on specific recognition of protospacer adjacent motifs (PAMs) for efficient binding and cleavage, and thereby avoid any accidental and lethal self-targeting of the CRISPR array. The PAM requirements of disclosed system were analyzed using a library approach, in which a fully randomized 5-bp sequence is cloned directly adjacent to the target site (FIG. 2A); junction PCR and deep sequencing then allows for selective amplification of successful integration products and comparison of enriched PAM motifs to the starting input library.

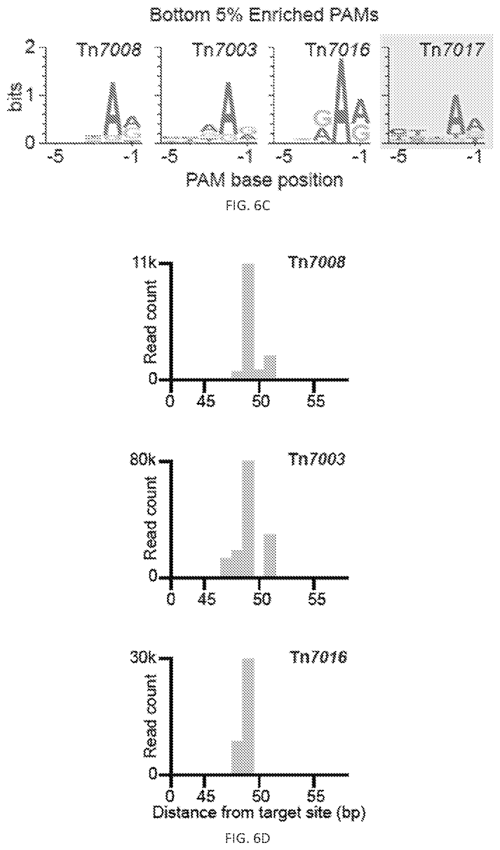

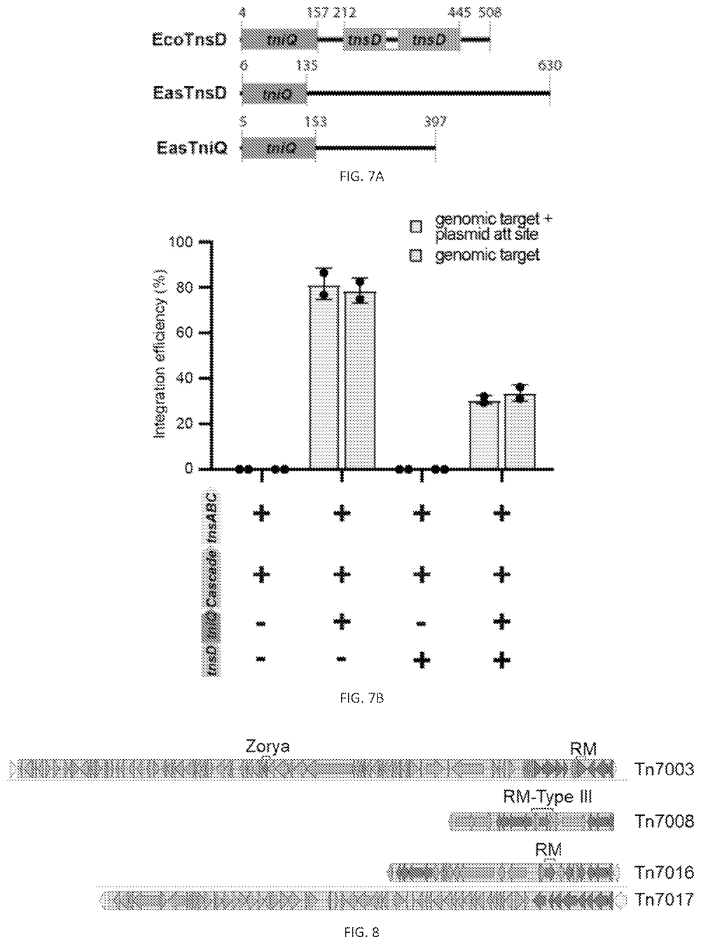

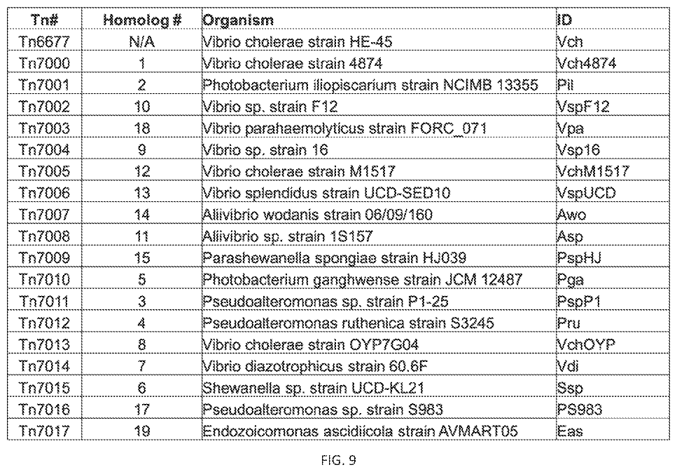

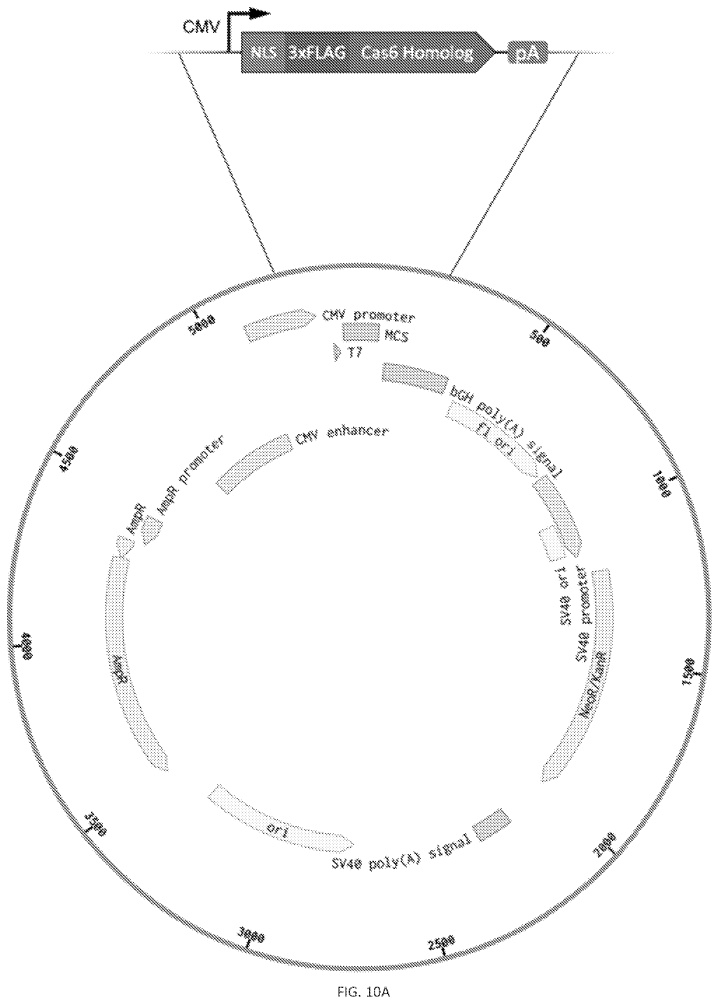

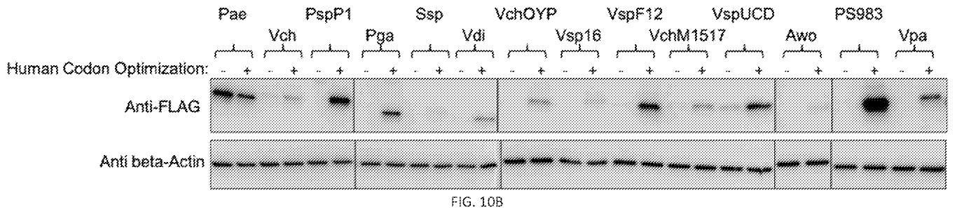

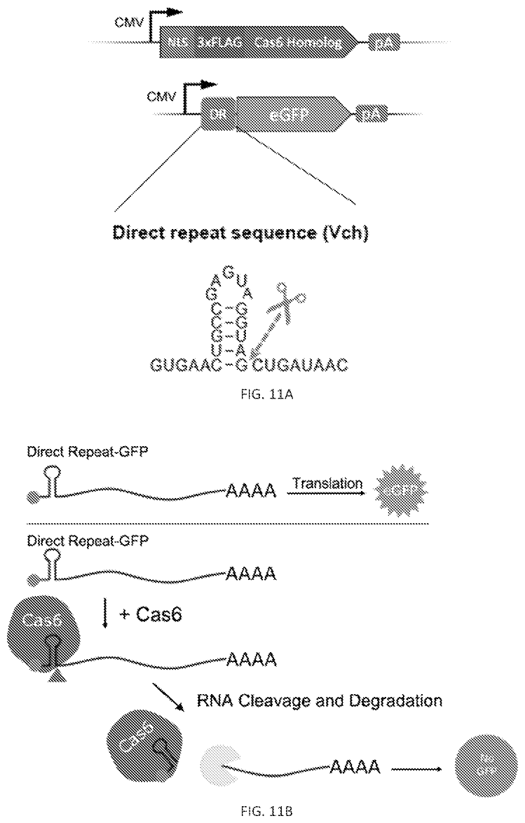

Interestingly, PAM enrichment scores were narrowly distributed and failed to reveal a strongly enriched or depleted group of sequence motifs (FIGS. 2B and 6A). A PAM motif for I-F3 systems was unable to be assessed using standard enrichment thresholds applied for other CRISPR-Cas effectors, and instead sequences found within the top and bottom 5% enriched sequences were analyzed. PAMs enriched in the upper 5% exhibited a clear ‘CN’ preference. Integration events for all CRISPR-Tn homologs occurred 48-52 nts downstream of the target site for substrates bearing a ‘CC’ PAM (FIGS. 2D and 6D). PAM sequences found in the lower 5% exhibited a ‘AN’ motif, which bears similarity to the ‘self’ sequence adjacent to the spacer sequence within these transposon-encoded CRISPR arrays (‘AC’ in most cases) (FIG. 6C). The presence of ‘self” PAMs in the output library suggested that transposition should be able to occur downstream of the CRISPR array itself, albeit at lower efficiency.

To validate these PAM library results, the integration efficiency of Tn7016 was measured for individual ‘CN’ and ‘NC’ PAMs within the same target plasmid context (FIG. 2D). These data revealed that plasmids with any CN PAM could be indistinguishably targeted for transposition, in excellent agreement with the library results. Tn7016 exhibited nearly PAM-less activity, with only a modest 2-fold decrease in activity at the ‘AC’ PAM.

Stringent PAM recognition is thought to accelerate the target search process, as is required during phage infections, rapid targeting kinetics during transposition is less likely to be selected for, whereas more permissive PAM recognition is well-suited to systems and organisms with evolutionary pressures. Flexible PAM recognition largely eliminates target site restrictions and may benefit genome engineering applications, analogously to recently engineered Cas9 variants that exhibit near PAM-less editing activity (See, Gasiunas et al., (2020) Nat Commun 17, 5512).

### Example 3

### Distinct TniQ Proteins Provide a Homing Pathway for Diverged CRISPR-Tn

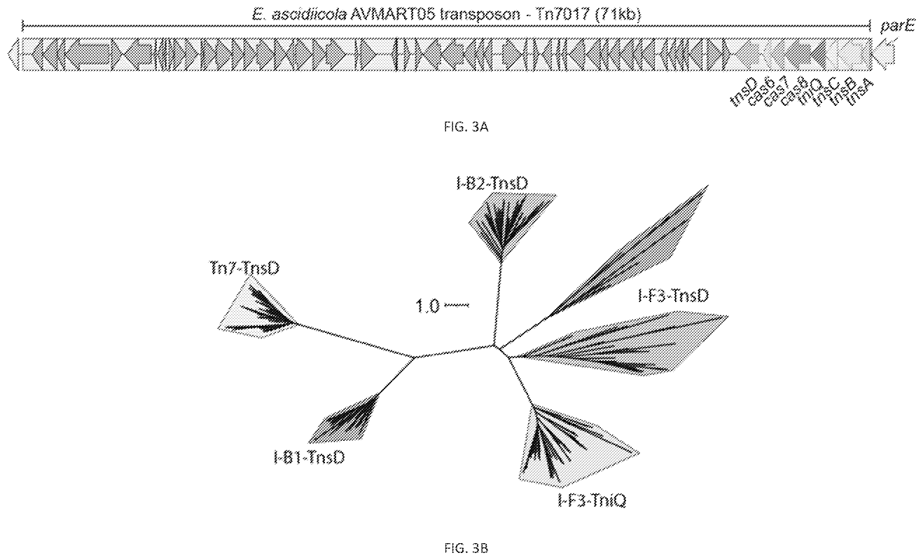

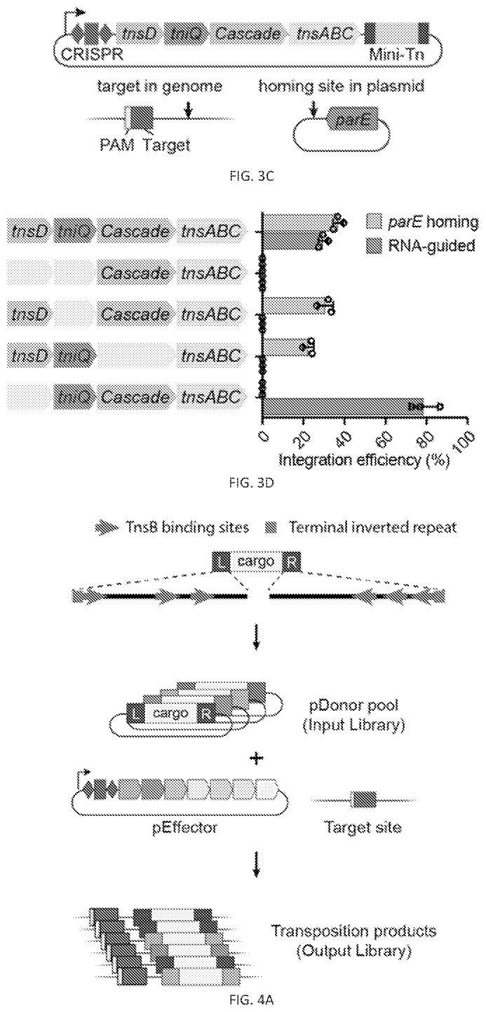

Tn7017 from an Endozoicomonas ascidiicola isolate, unusually included the presence of two distinct tniQ family genes (FIG. 3A). One gene is within the same operon as cas8-cas7-cas6 and encodes a TniQ protein with 397 amino acids, similar to other known TniQ proteins, whereas the other homolog is encoded on its own operon downstream of the CRISPR array and is much larger, 630 aa. Tn7017 may encode two distinct homing pathways that rely on alternative TniQ family proteins: an RNA-dependent pathway that exploits EasTniQ-Cascade for RNA-guided DNA target binding to promote horizontal transmission, and an RNA-independent pathway that exploits EasTnsD for sequence-specific DNA attachment site targeting to promote vertical transmission. Phylogenetic analysis revealed that EasTniQ was more closely related to TniQ proteins involved with RNA-guided transposition (FIG. 3B), while Eas-TnsD showed little sequence homology to TniQs from other RNA-guided CRISPR-Tn. Tn7017 was the only CRISPR-Tn system in the set that lacked an identifiable CRISPR array that could explain the insertion of Tn7017 downstream of the highly conserved parE gene (FIG. 5C).

A target plasmid (pTarget) with the 3′ end of the E. ascidiicola parE gene, which contains the anticipated EasTnsD binding site, was generated and transposition to pTarget (RNA-independent) and a genomic target site (RNA-dependent) was monitored in parallel (FIG. 3C). Transposition was indeed directed to both target sites, with the insertion site downstream of parE recapitulating the native genomic location of Tn7017. Gene deletions showed that integration into pTarget required EasTnsD but proceeded independently of Cascade, demonstrating that TnsABCD constitutes an independent targeting pathway directed at the parE safe harbor locus. In contrast, EasTniQ was necessary for the RNA-guided transposition pathway but functioned only when combined with Cascade. Interestingly, RNA-guided transposition efficiency at the genomic target increased drastically when EasTosD was omitted, whether or not pTarget was present (FIG. 3C), suggesting that EasTnsD may somehow inhibit TniQ-Cascade formation or compete for binding downstream transposase components.

Collectively, these data provide evidence of a type I-F3 CRISPR-Tn system that leverages two ThiQ-family proteins for distinct targeting pathways.

### Example 4

### CRISPR-Tn Systems are Orthogonal

Pooled library transposition assays were performed, in which pEffector plasmids were reacted with 20 pDonor substrates in a single transformation step (FIG. 4A). Successful integration products were then deep sequenced, and comparison to the starting library yielded enrichment scores describing the relative activity between each mini-Tn and the protein components from a given CRISPR-Tn system.

Pooled library transposition results revealed hotspots of integration activity, with most effectors acting upon only a narrow range of mini-Tn substrates. Intriguingly, Tn7017 could not be acted upon by any pEffector in the collection, aside from their cognate pairing, which in this case were not tested because experiments were performed at 37° C.′ and not the more optimal 25° C. As expected, the RNA-guided transposase machinery was most active on its own cognate transposon ends.

Orthogonal CRISPR-Tn systems allow for genomic target sites to be efficiently retargeted for the generation of tandem DNA insertions, without any repressive target immunity-like effect. E. coli Tn7 has been shown to prevent multiple insertions at the same target site through the action of TnsB and TnsC (Stellwagen and Craig, 1997). The integration efficiency of orthogonal CRISPR-Tn systems in E. coli strains that either lacked any pre-existing transposon or contained a mini-transposon derived from Tn6677 downstream of the same site being targeted by the orthogonal system were compared. Unlike the target immunity data with Tn6677, where the efficiency of a second insertion was close to 0%, orthogonal CRISPR-Tn systems generated a second insertion with the same efficiency, regardless of the presence of mini-Tn6677 (FIG. 4B). Transposase-transposon DNA sequence specificity dictated both transposition activity and target immunity effects, thus providing a straightforward opportunity to leverage multiple orthogonal CRISPR-Tn systems for high-efficiency genomic DNA integration in a given bacterial strain without spatial restrictions.

### Example 5

### CRISPR-Tn Systems for Mammalian Expression

A set of CRISPR-Tn systems that encode nuclease-deficient type I-F CRISPR-Cas systems and catalyze robust RNA-guided DNA integration activity in E. coli are outlined in FIG. 9, with the species and strain from which they derive, a numbering system, a numeric Tn#identifier for the native transposon from which the molecular components derive, and a unique ID for labeling purposes. Using these systems, alongside the system encoded by the transposon Tn6677 found in Vibrio cholerae strain HE-45, mammalian expression vectors were generated for the various components (Tables 4-7).

### Example 6

### Guide RNA Processing Activity by Cas6 in Human Cells

A panel of expression vectors were generated for the Cas6 subunit of type I-F Cascade (previously known as Csy4), in which the gene was placed downstream of a human cytomegalovirus (CMV) promoter within the backbone of a pcDNA3.1-derivative vector (FIGS. 10A-10B). Similar expression vectors were generated for Cas6 homologs derived from the additional CRISPR-Tn systems outlined in FIG. 9, and expression vectors encoding either Cas6 using the original gene sequence from the bacterial genomic source (e.g., with native codon usage), or a human codon-optimized gene sequence in which codon optimization was applied for human cell expression were generated. In additional embodiments, nuclear localization signals (NLS) are appended to either the N-terminus of Cas6, the C-terminus of Cas6, or both termini of Cas6 (Table 4).

Cas6, and/or other components, were expressed heterologously in human cells using standard methods. In a typical human cell transfection, approximately 50,000 HEK293T cells (maintained in DMEM media with 10% heat-inactivated FBS and penicillin-streptomycin) were seeded per well in a 24-well tissue culture plate coated with Poly-D-Lysine, 24 hours prior to transfection. The following day, cells were transfected with the desired plasmid(s) and Lipofectamine 2000 (Thermo Fisher) per the manufacturer's instructions. A transfection mix typically has approximately 1 μg of total DNA, with all transfection mixes in a given experiment containing equivalent mass amounts of total plasmid DNA; pUC19 may be used to normalize plasmid amounts, as needed. If analysis via flow cytometry will be performed, a fluorescent expression plasmid was included, which may be BFP, GFP, or mCherry, depending on the assay. This fluorescent plasmid was included as a transfection marker, such that flow-cytometry based gating for transfected cells can be performed before further analysis Cells were cultured at 37° C. with 5% CO₂, the media was replaced approximately 24 hours after transfection, and cells are harvested for analysis 48-72 hours post-transfection.

To test for and optimize Cas6 expression in human cells, HEK293T cells were transfected with various Cas6 expression vectors containing a 3×FLAG tag, cultured cells for 48-72 hours post-transfection, harvested the cell lysate, and used Western Blotting with anti-FLAG antibodies to assess Cas6 expression; anti-beta-actin antibodies are used as loading controls. Representative expression data are shown in FIG. 10B, indicating that native codon usage results in low expression levels across homologs, and codon optimization generates robust Cas6 expression.

Cas6 is a subunit of type I-F Cascade and is known to be a ribonuclease that binds to a stem-loop sequence encoded by the CRISPR repeat and cleaves at the base of the stem, this processing activity generates a mature form of CRISPR RNA (crRNA), or guide RNA, from a precursor form in which the spacer (guide) region is flanked by two copies of the repeat (Sternberg et al., RNA 18, 661-672 (2012)). In order to test for Cas6 ribonuclease activity in human cells, a GFP repression assay was developed, in which Cas6 activity can be directly monitored via a decrease or loss of GFP expression. Starting with a mammalian GFP reporter plasmid, a single copy of the full-length 28-bp CRISPR repeat (derived from the Tn6677-encoded CRISPR array) was introduced into the 5′-untranslated region (UTR), upstream of the GFP start codon but downstream of the transcription start site. Upon transcription, the mRNA will contain a stem-loop within the 5′-UTR recognized by Caso, and upon cleavage, the downstream coding sequence (CDS) for GFP is severed from the 5′-cap structure, leading to rapid degradation of the transcript and loss of GFP expression and fluorescence (FIGS. 11A-11B).

Starting with a representative Cas6 homolog derived from a canonical Type I-F1 CRISPR-Cas system from Pseudomonas aeruginosa (hereafter also referred to as “Pae”), transfection of HEK293T cells with both the Cas6 expression plasmid and GFP reporter plasmid yielded a significant decrease in GFP mean fluorescence intensity (MFI), as shown in FIGS. 11C-11D.

Cas6 derived from V. cholerae HE-45 Tn6677 (VchINTEGRATE) was tested and transfection with both the Vch Cas6 expression plasmid and the GFP reporter plasmid containing a Vch-derived CRISPR repeat yielded a significant decrease in GFP MFI compared to HEK293T cells transfected with only the GFP reporter plasmid (FIG. 11C). Placement of C-terminal motifs (e.g., NLS and/or 2A motifs) dramatically reduced the observed GFP repression, as shown in FIG. 11D.

Additional Cas6 homologs derived from homologous CRISPR-Tn systems were tested using a similar approach, wherein the VchINTEGRATE CRISPR repeat upstream of the GFP reporter gene was replaced with the CRISPR repeat sequence derived from the associated transposon-encoded CRISPR array (Table 4). Using the same flow cytometry assay and analysis, Cas6 variants with codon optimization, which also contained an SV40NLS-3×FLAG sequence appended to the N-terminus, exhibited a range of GFP repression activity (FIG. 11E). Thus, Cas6 homologs encoded by type I-F CRISPR-Tn systems were active for CRISPR repeat cleavage and gRNA processing in human cells.

### Example 7

### Transposon DNA Binding Activity by TnsB

TnsB is a transposase within the DDE retroviral integrase family of enzymes, which catalyzes the transesterification reaction upon integration of the transposon DNA into its target site during transposition. TnsB is also a sequence-specific DNA binding protein that recognizes conserved binding sites present on both ends of Tn7- and Tn5053-like transposons, often referred to as left (L) and right (R) ends. These TnsB binding sites are present in multiple copies on both ends, and are similar but not identical in sequence to each other. Previous studies suggest that formation of a paired-end complex between both transposon ends on the donor DNA molecule, as well as interactions with the targeting machinery on the target DNA molecule, trigger both the nuclease activity of TnsB, which leads to cleavage at the 3′ ends of both strands of transposon DNA, as well as the transesterification activity of TnsB that catalyzes attack of the liberated 3′-hydroxyl ends of the transposon DNA on the phosphate groups of the target DNA. However, in the absence of all of these molecular cues, TnsB still exhibits high-affinity binding to the TnsB binding sites on the transposon ends.

A fluorescence-based mammalian reporter assay was developed in HEK293T cells to study sequence-specific binding of TnsB to its cognate binding sites in mammalian cells. A tdTomato reporter gene was cloned downstream of a minimal CMV promoter, such that the basal expression level of tdTomato was low. When cells were co-transfected with this reporter plasmid and a plasmid encoding a nuclease-dead version of S. pyogenes Cas9 (e.g., dCas9) fused to a transcriptional activation domain, such as VP64, together with a plasmid encoding a guide RNA targeting a DNA sequence immediately upstream of the minimal CMV promoter, the localized transcriptional activation domain led to a potent increase in RNA Polymerase II recruitment and tdTomato transcription. This synthetic transcriptional activation resulted in a quantifiable increase in the tdTomato fluorescence intensity of transfected cells, which is quantified by flow cytometry.

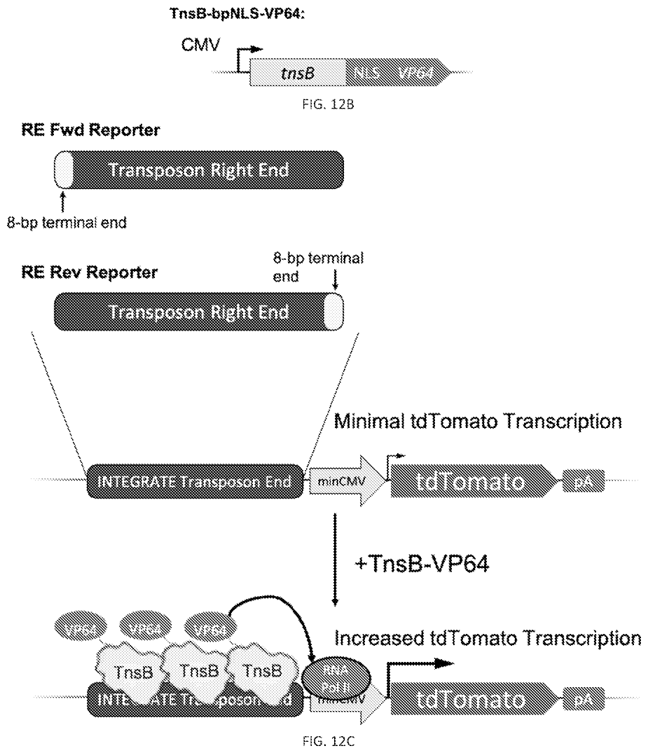

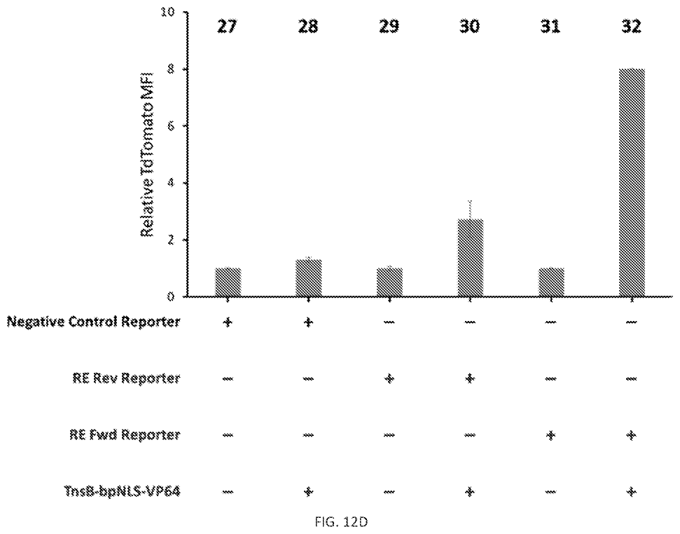

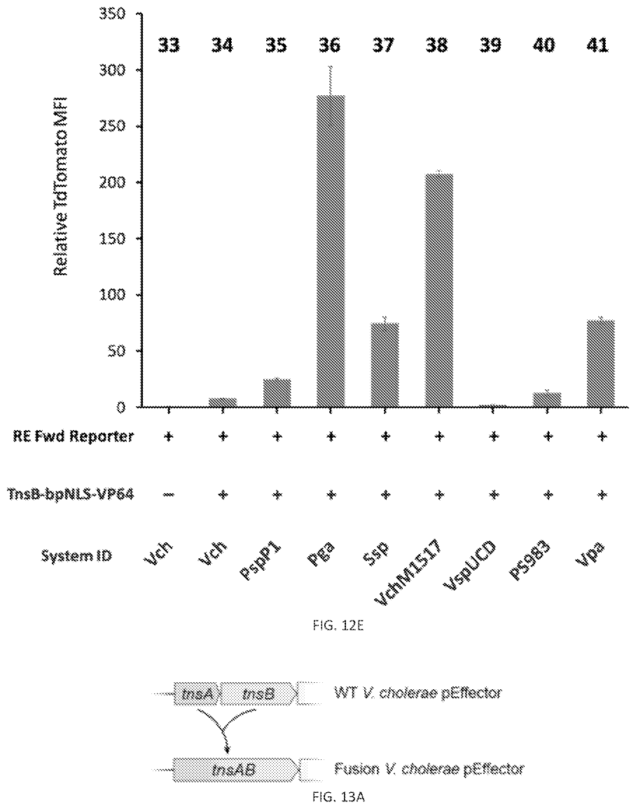

This approach was adapted to monitor TnsB binding by cloning a panel of transposon end substrates derived from Tn6677 (VchINTEGRATE) directly upstream of the minimal CMV promoter on the reporter plasmid, and by cloning a similar VP64 transcriptional activation domain onto the C-terminus of VchInsB (FIGS. 12B-12C; see plasmids in Table 5). A variety of different reporter plasmid constructs were tested, including transposon right end constructs that were inserted in opposite orientations (Fwd and Rev) relative to the minimal CMV promoter. When HEK293T cells were co-transfected with the modified reporter plasmid and the TnsB-VP64 activator plasmid, a robust increase in cellular tdTomato fluorescence was observed, which was strongest for reporter plasmids in which the transposon end was oriented such that the 8-base pair (bp) terminal end was distal to the minimal CMV promoter (FIGS. 12C-12D). In control experiments, this transcriptional activation activity was lost when the transposon end substrate was replaced with a non-targeting sequence, such that no TnsB binding was expected to occur (FIG. 12D).

Additional TnsB homologs derived from CRISPR-Tn systems were tested using a similar approach with transposon end sequences derived from the associated homologous transposon system. Using the same flow cytometry assay and analysis, TnsB variants exhibited a range of tdTomato activation activity, demonstrating that CRISPR-Tn systems encode TnsB proteins with variable DNA binding activity in mammalian cell applications (FIG. 12E).

### Example 8

### TnsA-TnsB Fusion Protein for RNA-Guided DNA Integration

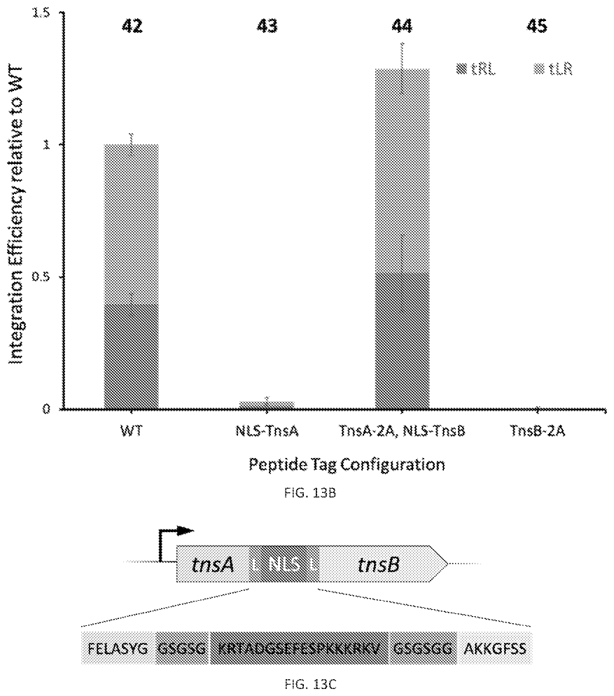

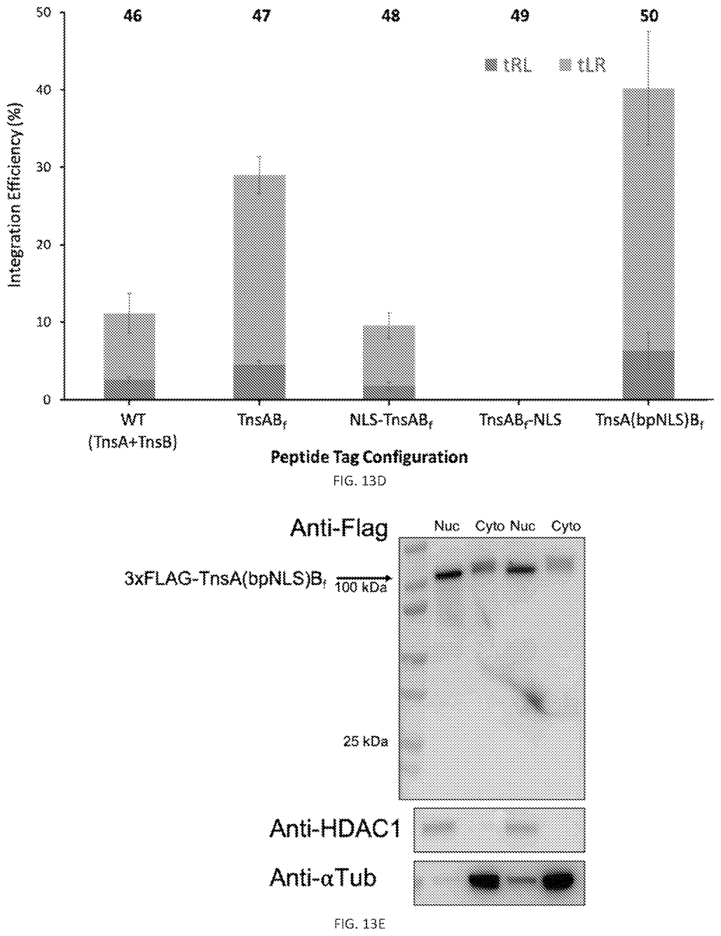

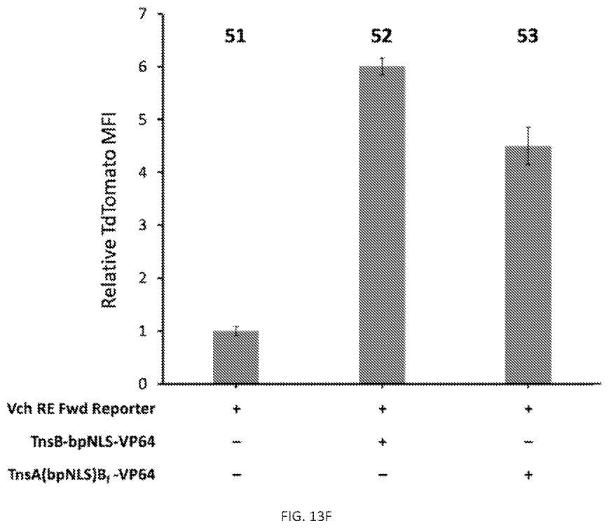

Two of the type I-F CRISPR-Tn systems shown in FIG. 1 encode natural fusion polypeptides between the endonuclease-family TnsA protein and the DDE transposase-family TnsB protein: Tn7007 derived from Aliivibrio wodanis strain 06/09/160 and Tn7009 derived from Parashewanella spongiae strain HJ039. These CRISPR-Tn systems are active for RNA-guided DNA integration in an E. coli host, and based on these natural fusion polypeptides, a functional engineered fusion of TnsA-TnsB derived from Tn6677 from V. cholerae strain HE-45 was designed (FIG. 13A; Vo et al., bioRxiv 1-17 (2021), doi: 10,1101/2021.02.11.430876). This fusion polypeptide, referred to as TnsAB, maintained wild-type RNA-guided DNA integration activity in E. coli, as compared to experiments in which TnsA and TnsB were separately expressed.

In order to leverage TnsABf in mammalian cells for nuclear integration activity, in one embodiment, a nuclear localization signal may be appended to the fusion protein in order to promote nuclear trafficking. In the context of separate expression of TnsA and TnsB, TnsA and TnsB activity were previously shown to be sensitive to terminal NLS tagging. Specifically, when modified variants of VchINTEGRATE were tested in E. coli for genomic RNA-guided DNA integration, either an N-terminal NLS on TnsA, or a C-terminal NLS on TnsB, led to severe reductions in integration efficiency, as compared to their untagged counterparts (FIG. 13B).

A bacterial expression plasmid encoding InsABf with an internal bipartite NLS tag inserted directly in frame with both TnsA and TnsB, in the region in between the native polypeptide sequences, was engineered. In addition, short glycine-serine linkers were also inserted in front of, and behind, the BP-NLS tag. The design is schematized in FIG. 13C, and plasmid descriptions are found in Table 5. The internal NLS tag not only did not adversely impact integration activity, but that it in fact increased total integration efficiency relative to the positive control containing separately encoded TnsA and TnsB (FIG. 13D).

A mammalian expression vector encoding a similarly designed TnsAB_{f} polypeptide but with human codon-optimized gene sequences was designed. An N-terminal epitope tag was added and cells were transfected with the TnsABf expression plasmid. Western blotting confirmed that the TnsAB_{f} fusion polypeptide was highly expressed, successfully trafficked to the nucleus, and persisted in its full-length form, indicating an absence of detectable degradation or proteolysis of the fusion polypeptide (FIG. 13E). To confirm that the TnsABt polypeptide was functional for transposon end binding, similar tdTomato activation assays, as described previously, were employed using a VP64-TnsABf construct, and tdTomato was activated in a TnsB binding site-dependent fashion (FIG. 13F).

### Example 9

### RNA-Guided DNA Integration in Human Cells Using Vch INTEGRATE

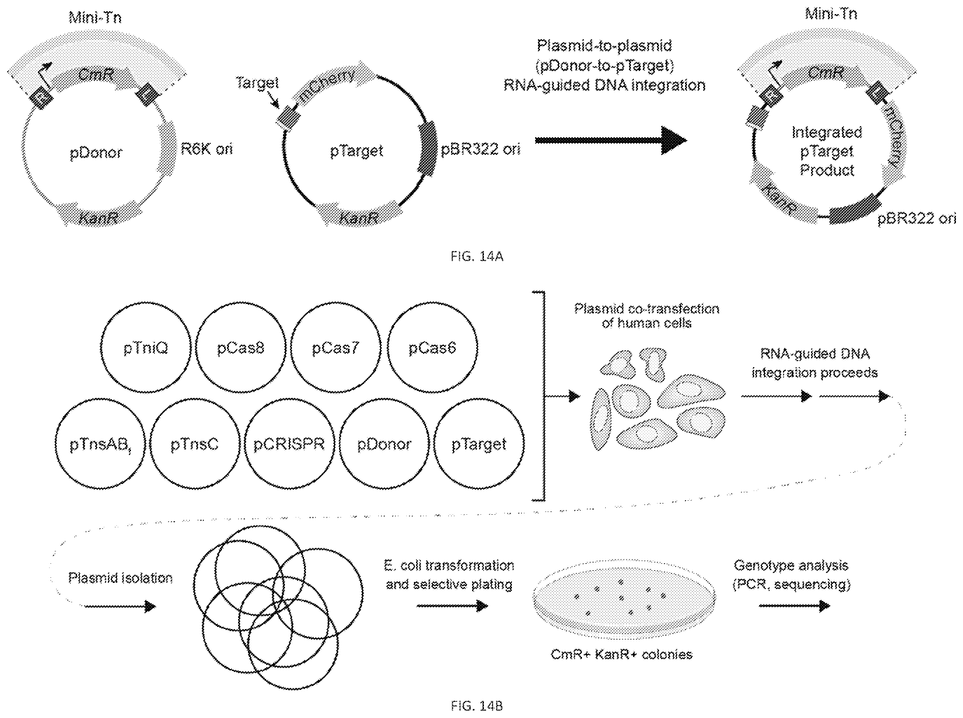

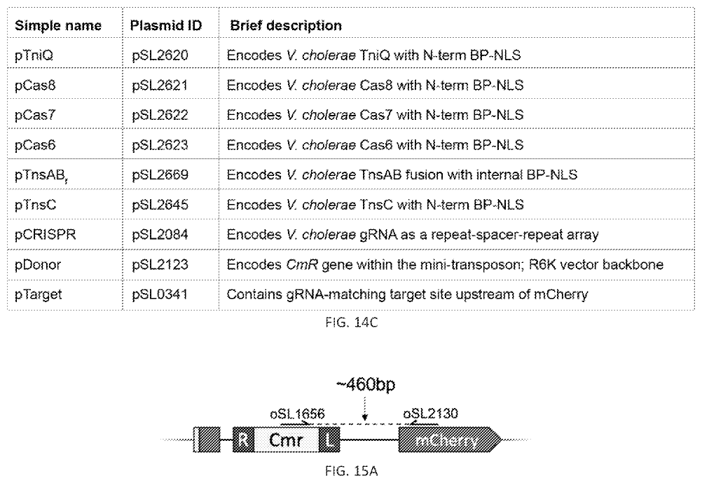

A plasmid-based transposition assay was adapted in order to reconstitute RNA-guided DNA integration in human cells (FIG. 14A) by using the modified expression vectors mentioned elsewhere herein. The assay comprised co-transfection of all of the necessary protein expression vectors (TniQ, Cas8, Cas7, Cas6, TosC, and TnsABf), a vector encoding gRNA, a donor DNA vector (pDonor), and a target DNA vector (pTarget). If cut-and-paste transposition occurred within the transfected cells, a new plasmid in which the mini-transposon present on pDonor is integrated into the pTarget plasmid, downstream of the 32-bp target site complementary to the gRNA sequence would result. Plasmid DNA was isolated from the transfected human cells after 48-72 hours of growth post-transfection and used to transform E. coli; successful transposition events were identified based on the characteristic antibiotic resistance genes present on the backbone and within the mini-transposon donor DNA substrate itself, as described further below. Alternatively to this phenotypic assay, the isolated plasmids may be tested directly for the presence of integrated pTarget product, based on unique and characteristic junction PCR products specific to the expected transposition product. In control experiments, the gRNA sequence was replaced with a non-targeting (scrambled) control; and/or the pTarget plasmid may also be modified to eliminate the target site; and/or one or more expression vectors may be omitted from the transfection mix.

A pDonor variant was cloned onto the non-replicative R6K origin, which can be maintained in a pir+ strain of E. coli, but which fails to replicate and stably transform most standard laboratory E. coli cloning strains. The pDonor encoded a kanamycin resistance gene (KanR) on the backbone, as well as a promoter-driven chloramphenicol resistance gene (CmR) within the mini-transposon itself. The target plasmid contained the same mCherry expression vector, with a gRNA-target site pairing that led to highly efficient TniQ-Cascade and TnsC-based transcriptional activation. pTarget also encoded a standard KanR gene on the backbone, and the remaining protein and gRNA expression plasmids encoded a standard ampicillin resistance gene (AmpR) on the backbone. The plasmid mixture obtained from transfected human cells—which contained unreacted pDonor and pTarget, as well as integrated pTarget product DNA—was isolated, commercial NEB 10-beta E. coli electrocompetent cells were transformed, and the cells were plated on LB-agar plates containing either chloramphenicol alone (25 μg/mL) or both chloramphenicol (25 μg/mL) and kanamycin (50 μg/mL). Because pDonor cannot replicate in 10-beta E. coli cells, due to the R6K backbone, the primary source of kanamycin-and chloramphenicol-resistant colonies were cells that were transformed with pTarget (KanR) which also received the mini-transposon encoding CmR. The overall strategy is outlined in FIGS. 14A-14B.

HEK293T cells were transfected with the plasmid mixtures shown in FIG. 14C using Lipofectamine 2000 and standard protocols. Cells were cultured at 37° C. with 5% CO₂, the media was replaced approximately 24 hours after transfection, and cells were harvested for analysis 48-72 hours post-transfection. The transfected plasmids were purified using the Qiagen Miniprep kit per the manufacturer's instructions, and further concentrated using the Qiagen MinElute column. Of this final purified plasmid mixture, 1 μl was used to electroporate NEB 10-beta electrocompetent E. coli cells (NEB) per the manufacturer's instructions. After recovery at 37° C., cells were plated onto LB-agar plates containing chloramphenicol. Chloramphenicol-resistant colonies were then replated onto new LB-agar plates containing both chloramphenicol and kanamycin. Chloramphenicol and kanamycin-resistant colonies were then harvested for genotypic analyses.

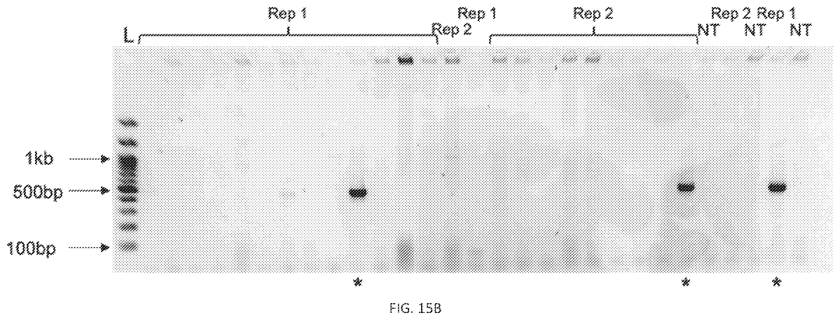

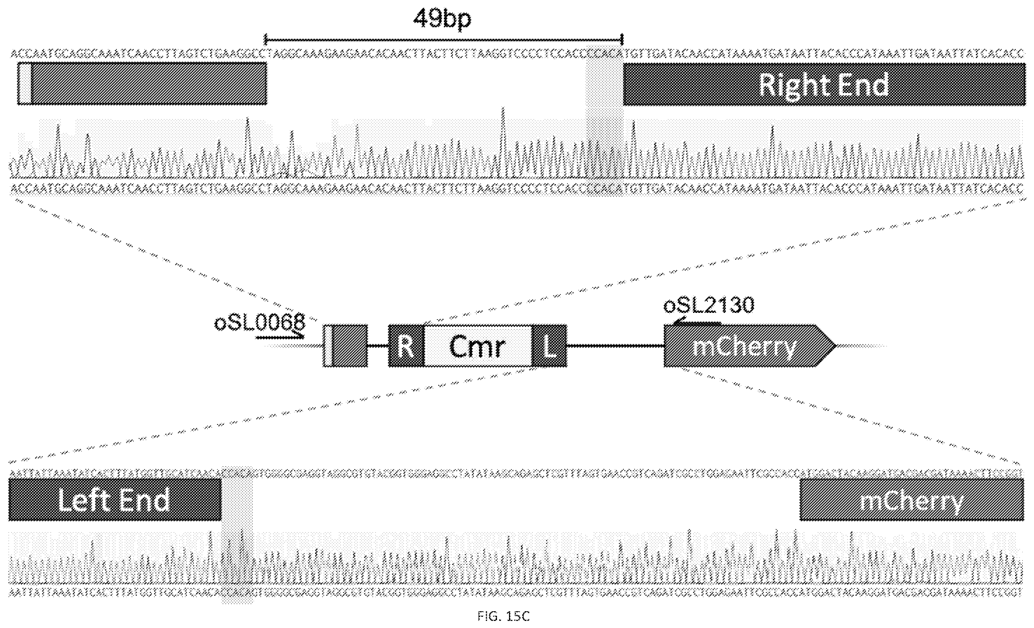

A low level of background CmR+ colonies were observed in experiments using a non-targeting gRNA, which were negative for donor DNA integration events. However, two biological replicates of transfection experiments using a targeting gRNA matching pTarget, after plasmid isolation and E. coli transformation, yielded an increased number of CmR+ colonies. Analytical PCR on biological material isolated from these colonies was completed using a primer pair in which one primer was specific to a region within the mini-transposon itself, and a second primer was specific to a constant region within the pTarget backbone, proximal to the anticipated integration site (FIG. 15A). PCR reactions were performed using NEB OneTaq DNA Polymerase, and reactions were analyzed by agarose gel electrophoresis. Three distinct colonies across the two biological replicates yielded robust amplicons, with DNA bands migrating at the expected size (˜460 bp) for the anticipated junction PCR product (FIGS. 15A-15B). One of the colonies that produced a junction PCR product amplicon underwent Sanger sequencing analysis with primers that would read across both junctions within pTarget. The resulting sequencing chromatograms clearly revealed the presence of bona fide integration products, in which the mini-Tn was present 49-bp downstream of the 3′ edge of the target site (FIG. 15C) Furthermore, when comparing sequencing information on both junctions, a precise duplication of 5-bp was found, in line with the 5-bp target-site duplication (TSD) generated by transposition events with Tn7-like transposons (FIG. 15C).

### Example 10

### Alternative Guide RNA Expression Vectors for RNA-Guided DNA Integration

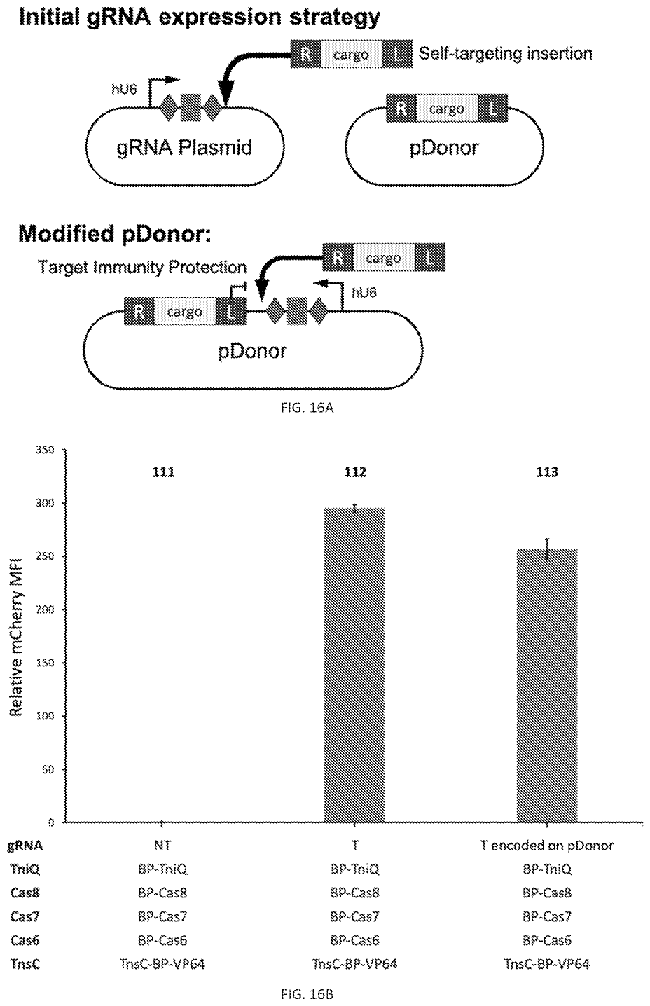

Canonical approaches for exploiting CRISPR-Cas systems for genome editing, including the vast majority of CRISPR-Cas9 methods, encode the guide RNA downstream of an RNA Polymerase III U6 promoter. Within the context of CRISPR-Tn systems such as VchINTEGRATE, expression of the guide RNA on a separate plasmid separate from the mini-transposon donor DNA leads to a risk of self-targeting, as previously described (Vo et al., Nature Biotechnology 39, 480-489 (2021)). Self-targeting could reduce the efficiency of the overall system by inactivating a select pool of expression vectors, and could also lead to undesirable integration events. In order to avoid this, a new donor DNA plasmid (pDonor) was designed that encodes the guide RNA downstream of an RNA Polymerase III U6 promoter immediately adjacent to the mini-transposon donor itself (FIG. 16A). This approach leverages the natural mechanism of target immunity to ‘privilege’ the CRISPR array and prevent self-targeting, leading to proper RNA-guided DNA integration at the intended genomic target site. To verify that this strategy could be similarly adopted in mammalian cells, gRNA function was tested in the context of transcriptional activation assays relying on TnsC—BP-VP64 fusion proteins (FIG. 16B). Targeting gRNA encoded on pDonor led to nearly indistinguishable levels of transcriptional activation, as the exact same gRNA encoded on its own plasmid separate from pDonor.

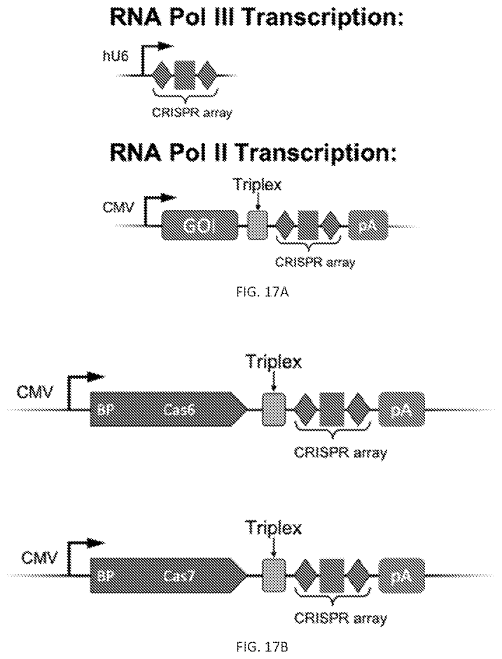

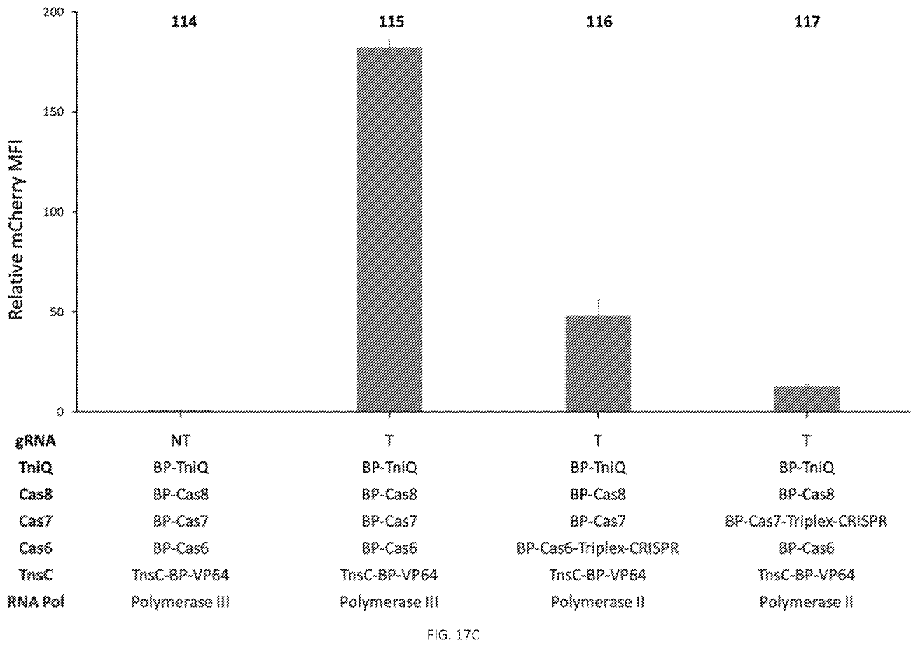

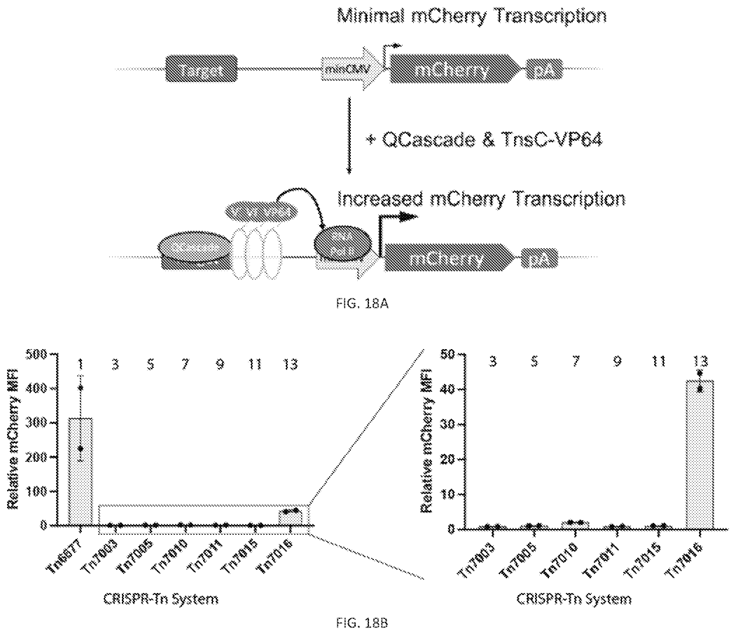

Vectors were designed in which both a VchINTEGRATE protein component and guide RNA were encoded as a type of polycistronic construct on the same RNA molecule, controlled by an RNA Pol II promoter. This strategy reduced the number of separate plasmids required for transfection in order to reconstitute the full INTEGRATE system, and it also promoted cytoplasmic TniQ-Cascade complex formation by exporting the gRNA to the cytoplasm where protein components are initially expressed and localized, prior to nuclear trafficking (FIG. 17A). Cytoplasmic assembly of TniQ-Cascade also obviated the need to place NLS tags on every single protein subunit, since a select few NLS tags on the multi-subunit TniQ-Cascade complex would be sufficient for the entire complex to efficiently traffic to the nucleus. A 110-bp fragment from the MALAT1 locus, previously shown to stabilize mRNA transcripts lacking a PolyA tail (Nissim et al., Mol Cell 54, 698-710 (2014)), was designed and encoded downstream of a gene of interest, in between the stop codon and the CRISPR array. In this context, the CRISPR array was found within the 3′-UTR. Cas6 processing of the pre-crRNA leads to cleavage of the fusion mRNA-crRNA species, but the triplex structure protects the protein-coding mRNA from 3′ exonuclease-based degradation once the poly(A) tag has been severed from the rest of the transcript. Two constructs were designed, in which the MALAT1 triplex sequence and CRISPR array were encoded within the 3′ UTR of either a BP NLS-tagged Cas6 or Cas7, and the ability of these modified gRNA expression cassettes to function for RNA-guided DNA targeting and synthetic transcriptional activation was measured using TnsC—BP-VP64 activators (FIG. 17B). These alternative gRNA expression contexts were functional for transcriptional activation, albeit with slightly reduced efficiency as compared to a separate plasmid encoding the gRNA on a Pol III transcript (FIG. 17C). The CRISPR array may be placed within other 3′-UTRs, such as drug resistance of fluorescence reporter protein genes, and the protein machinery may be further modified in order to optimize the formation of TniQ-Cascade in the cytoplasm.

### Example 11

### Cas7 as Mediator of Efficiency

To test if modifying the relative concentrations of each plasmid that is co-transfected for TnsC-based transcriptional activation may further improve RNA-guided targeting, and subsequent integration, various ratios of components were tested in a transcriptional activation assay. Various permutations of Cas7, including multiple tandem BP NLS tags, and/or combinations of NLS tags and 3×FLAG epitope tags were tested and transcriptional activation activity was substantially increased when only Cas7 was switched from an SV40 NLS to a BP NLS, and that a 2×BP-NLS tag slightly increased transcriptional activation. In contrast, the addition of more BP-NLS tags led to a decrease of transcriptional activation.

The relative concentration of a Cas7 expression plasmid was increased compared to all other components, and a dose-dependent increase in activation was seen using a similar transcriptional activation assay. Increases in the relative concentration of other subunits resulted in limited increases in transcriptional activation, and in some cases a reduction in transcriptional activation.

TABLE 3
Table of plasmids used for the transformation
and transfection experiments

| Expt. | Type of | |
|---|---|---|
| ID | experiment | Plasmid(s) Used |
| 1 | Transfection | pSL1657, pSL2278 |
| 2 | Transfection | pSL1657, pSL2278, pSL2281 |
| 3 | Transfection | pSL1657, pSL2277, pSL2069 |
| 4 | Transfection | pSL1657, pSL2277, pSL1490 |
| 5 | Transfection | pSL1657, pSL2277, pSL2067 |
| 6 | Transfection | pSL1657, pSL2277, pSL2307 |
| 7 | Transfection | pSL1657, pSL2277, pSL1198 |
| 8 | Transfection | pSL1657, pSL2277, pSL2283 |
| 9 | Transfection | pSL1657, pSL2278, pSL1061 |
| 10 | Transfection | pSL1657, pSL2278, pSL2311 |
| 11 | Transfection | pSL1657, pSL2277, pSL2067 |
| 12 | Transfection | pSL1657, pSL2327, pSL2508 |
| 13 | Transfection | pSL1657, pSL2328, pSL2509 |
| 14 | Transfection | pSL1657, pSL2329, pSL2510 |
| 15 | Transfection | pSL1657, pSL2330, pSL2511 |
| 16 | Transfection | pSL1657, pSL2331, pSL2512 |
| 17 | Transfection | pSL1657, pSL2335, pSL2513 |
| 18 | Transfection | pSL1657, pSL2333, pSL2514 |
| 19 | Transfection | pSL1657, pSL2334, pSL2515 |
| 20 | Transfection | pSL1657, pSL2335, pSL2516 |
| 21 | Transfection | pSL1657, pSL2336, pSL2517 |
| 22 | Transfection | pSL1657, pSL2337, pSL2518 |
| 23 | Transfection | pSL1657, pSL2448, pSL2519 |
| 24 | Transfection | pSL1657, pSL2392, pSL2520 |
| 25 | Transfection | pSL1657, pSL2393, pSL2521 |
| 26 | Transfection | pSL1657, pSL2449, pSL2522 |
| 27 | Transfection | pSL0303, pSL2621, pSL2533 |
| 28 | Transfection | pSL0303, pSL2679, pSL2533 |
| 29 | Transfection | pSL2550, pSL2621, pSL2533 |
| 30 | Transfection | pSL2550, pSL2679, pSL2533 |
| 31 | Transfection | pSL2561, pSL2621, pSL2533 |
| 32 | Transfection | pSL2561, pSL2679, pSL2533 |
| 33 | Transfection | pSL2561, pSL2621, pSL2533 |
| 34 | Transfection | pSL2561, pSL2679, pSL2533 |
| 35 | Transfection | pSL2792, pSL2802, pSL2533 |
| 36 | Transfection | pSL2793, pSL2803, pSL2533 |
| 37 | Transfection | pSL2794, pSL2804, pSL2533 |
| 38 | Transfection | pSL2797, pSL2808, pSL2533 |
| 39 | Transfection | pSL2798, pSL2809, pSL2533 |
| 40 | Transfection | pSL2800, pSL2810, pSL2533 |
| 41 | Transfection | pSL2801, pSL2811, pSL2533 |
| 42 | Transformation | pSL0527, pSL0828, pSL0283 |
| 43 | Transformation | pSL0527, pSL0828, pSL1054 |
| 44 | Transformation | pSL0527, pSL0828, pSL1055 |
| 45 | Transformation | pSL0527, pSL0828, pSL1482 |
| 46 | Transformation | pSL0527, pSL0828, pSL0283 |
| 47 | Transformation | pSL0527, pSL0828, pSL1738 |
| 48 | Transformation | pSL0527, pSL0828, pSL2096 |
| 49 | Transformation | pSL0527, pSL0828, pSL2097 |
| 50 | Transformation | pSL0527, pSL0828, pSL2542 |
| 51 | Transfection | pSL2561, pSL2621, pSL2533 |
| 52 | Transfection | pSL2561, pSL2679, pSL2533 |
| 53 | Transfection | pSL2561, pSL2825, pSL2533 |
| 111 | Transfection | pSL0302, pSL0341, pSL2620, pSL2621, |
| | | pSL2622, pSL2623, pSL2783, pSL1409 |
| 112 | Transfection | pSL0302, pSL0341, pSL2620, pSL2621, |
| | | pSL2622, pSL2623, pSL2783, pSL2084 |
| 113 | Transfection | pSL0302, pSL0341, pSL2620, pSL2621, |
| | | pSL2622, pSL2623, pSL2783, pSL2945 |
| 114 | Transfection | pSL2533, pSL0341, pSL2620, pSL2621, |
| | | pSL2622, pSL2623, pSL2783, pSL1409 |
| 115 | Transfection | pSL2533, pSL0341, pSL2620, pSL2621, |
| | | pSL2622, pSL2623, pSL2783, pSL2084 |
| 116 | Transfection | pSL2533, pSL0341, pSL2620, pSL2621, |
| | | pSL2622, pSL2869, pSL2783 |
| 117 | Transfection | pSL2533, pSL0341, pSL2620, pSL2621, |
| | | pSL2871, pSL2623, pSL2783 |

TABLE 4
Description of plasmids for Cas6 expression
and activity assays in mammalian cells

| Plasmid | |
|---|---|
| ID | Plasmid name |
| pSL2333 | pcDNA5/FRT-DR-eGFP-D2PEST-NLS Homologue 9 |
| pSL2334 | pcDNA5/FRT-DR-eGFP-D2PEST-NLS Homologue 10 |
| pSL2335 | pcDNA5/FRT-DR-eGFP-D2PEST-NLS Homologue 12 |
| pSL2336 | pcDNA5/FRT-DR-eGFP-D2PEST-NLS Homologue 13 |
| pSL2337 | pcDNA5/FRT-DR-eGFP-D2PEST-NLS Homologue 14 |
| pSL2392 | pcDNA5/FRT-DR-eGFP-D2PEST-NLS Homologue 17 |
| pSL2393 | pcDNA5/FRT-DR-eGFP-D2PEST-NLS Homologue 18 |
| pSL2448 | pcDNA5/FRT-DR-eGFP-D2PEST-NLS Homologue 15 |
| pSL2449 | pcDNA5/FRT-DR-eGFP-D2PEST-NLS Homologue 19 |
| pSL2508 | pcDNA3.1_NLS_FLAG_HCO_Cas6_PspP1_Hom3 |
| pSL2509 | pcDNA3.1_NLS_FLAG_HCO_Cas6_Pru_Hom4 |
| pSL2510 | pcDNA3.1_NLS_FLAG_HCO_Cas6_Pga_Hom5 |
| pSL2511 | pcDNA3.1_NLS_FLAG_HCO_Cas6_Ssp_Hom6 |
| pSL2512 | pcDNA3.1_NLS_FLAG_HCO_Cas6_Vdi_Hom7 |
| pSL2513 | pcDNA3.1_NLS_FLAG_HCO_Cas6_VchOYP_Hom8 |
| pSL2514 | pcDNA3.1_NLS_FLAG_HCO_Cas6_Vsp16_Hom9 |
| pSL2515 | pcDNA3.1_NLS_FLAG_HCO_Cas6_VspF12_Hom10 |
| pSL2516 | pcDNA3.1_NLS_FLAG_HCO_Cas6_VchM1517_Hom12 |
| pSL2517 | pcDNA3.1_NLS_FLAG_HCO_Cas6_VspUCD_Hom13 |
| pSL2518 | pcDNA3.1_NLS_FLAG_HCO_Cas6_Awo_Hom14 |
| pSL2519 | pcDNA3.1_NLS_FLAG_HCO_Cas6_PspHJ_Hom15 |
| pSL2520 | pcDNA3.1_NLS_FLAG_HCO_Cas6_PS983_Hom17 |
| pSL2521 | pcDNA3.1_NLS_FLAG_HCO_Cas6_Vpa_Hom18 |
| pSL2522 | pcDNA3.1_NLS_FLAG_HCO_Cas6_Eas_Hom19 |

TABLE 5
Description of plasmids for TnsB expression
and activity assays in mammalian cells

| Plasmid | |
|---|---|
| ID | Plasmid name |
| pSL0283 | pCOLA_Vch_TnsA_TnsB_TnsC |
| pSL0303 | SP-cas9 human reporter 1 + 100-tdtomato |
| pSL0527 | pUC19_Vch_Tn7R_CmR_Tn7L |
| pSL0828 | pCDF_Vch_TniQ_Cascade_CRISPR(Target4_lacZ-690) |
| pSL1054 | pCOLA_Vch_TnsABC, NLS-TnsA |
| pSL1055 | pCOLA_Vch_TnsABC, TnsA-T2A, NLS-TnsB |
| pSL1482 | pCOLA_Vch_TnsABC, TnsB-T2A |
| pSL1738 | pCOLA_Vch_TnsAB(fusion)_TnsC |
| pSL2096 | pCOLA_Vch_NLS-TnsAB(fusion)_TnsC |
| pSL2097 | pCOLA_Vch_TnsAB(fusion)-NLS_TnsC |
| pSL2533 | p6A_Macrolab CMV_acGFP_noORI |
| pSL2542 | pCOLA_Vch_TnsAB(fusion)_internal-bpNLS_TnsC |
| pSL2550 | gRNA_tdTomato reporter 1-Transposon End Right |
| pSL2561 | gRNA_tdTomato reporter 1-Transposon Right End |
| pSL2621 | pcDNA3.1_hCO_Vch_BP-NLS-Cas8 |
| pSL2679 | pcDNA3.1_hCO_Vch_TnsB-BP-NLS_VP64 |
| pSL2792 | gRNA_tdTomato reporter 1-Transposon Right End_Hom3 |
| | PspP1 |
| pSL2793 | gRNA_tdTomato reporter 1-Transposon Right End_Hom5 |
| | Pga |
| pSL2794 | gRNA_tdTomato reporter 1-Transposon Right End_Hom6 |
| | Ssp |
| pSL2797 | gRNA_tdTomato reporter 1-Transposon Right End_Hom12 |
| | VchM1517 |
| pSL2798 | gRNA_tdTomato reporter 1-Transposon Right End_Hom13 |
| | VspUCD |
| pSL2800 | gRNA_tdTomato reporter 1-Transposon Right End_Hom17 |
| | PS983 |
| pSL2801 | gRNA_tdTomato reporter 1-Transposon Right End_Hom18 |
| | Vpa |
| pSL2802 | pcDNA3.1(+)_Hom3_P1-25_TnsB-BPNLS-VP64 |
| pSL2803 | pcDNA3.1(+)_Hom5_JCM12487 TnsB-BPNLS-VP64 |
| pSL2804 | pcDNA3.1(+)_Hom6_UCD-KL21_TnsB-BPNLS-VP64 |
| pSL2808 | pcDNA3.1(+)_Hom12_M1517_TnsB-BPNLS-VP64 |
| pSL2809 | pcDNA3.1(+)_Hom13_UCD-SED10_TnsB-BPNLS-VP64 |
| pSL2810 | pcDNA3.1(+)_Hom17_S983_TnsB-BPNLS-VP64 |
| pSL2811 | pcDNA3.1(+)_Hom18_FORC_071_TnsB-BPNLS-VP64 |
| pSL2825 | pcDNA3.1_hCO_Vch_TnsA_BP-NLS_TnsB-VP64 |

TABLE 6
Description of plasmids for TuiQ-Cascade and InsC
expression and activity assays in mammalian cells

| Plasmid ID | Plasmid name |
|---|---|
| pSL0302 | CAGG-eBFP2 |
| pSL0341 | mCherry reporter for CRISPRa |
| pSL1061 | pcDNA3.1_hCO_Vch_NLS-TnsA |
| pSL1198 | pcDNA3.1_hCO_Vch_NLS-Cas6-T2A |
| pSL1409 | p6A_Vch_hU6_CRISPR(tSL0105) |
| pSL1490 | pcDNA3.1_hCO_Vch_Cas6 |
| pSL2084 | p6A_Vch_hU6_CRISPR((SL0264) |
| pSL2533 | p6A_Macrolab CMV_acGFP_noORI |
| pSL2620 | pcDNA3.1_hCO_Vch_BP-NLS-TniQ |
| pSL2621 | pcDNA3.1_hCO_Vch_BP-NLS-Cas8 |
| pSL2622 | pcDNA3.1_hCO_Vch_BP-NLS-Cas7 |
| pSL2623 | pcDNA3.1_hCO_Vch_BP-NLS-Cas6 |
| pSL2783 | p6A_hCO_Vch_TnsC_BP-NLS-VP64 |

TABLE 7
Description of Plasmids for RNA Polymerase
II-based expression of INTEGRATE guide RNAs

| Plasmid | |
|---|---|
| ID | Plasmid name |
| pSL2869 | pcDNA3.1(+)_BP- |
| | NLS_VchCas6_Triplex_VchCRISPR_tSL0264 |
| pSL2871 | pcDNA3.1(+)_BP- |
| | NLS_VchCas7_Triplex_VchCRISPR_tSL0264 |
| pSL2945 | pUC19-RF-CMVe/p-PuroR-T2A-eGFP-BGH-LF_U6 |
| | tSL0264 |

### Example 12

### RNA-Guided DNA Integration in Human Cells

A plasmid-based transposition assay was adapted in order to reconstitute RNA-guided DNA integration in human cells, using the modified expression vectors mentioned elsewhere. The strategy relies on co-transfection of all of the necessary protein expression vectors (TniQ, Cas8, Cas7, Cas6, TnsC, and TnsABf), a vector encoding gRNA, a donor DNA vector (pDonor), and a target DNA vector (pTarget); cut-and-paste transposition occurs within the transfected cells, resulting in a new plasmid in which the mini-transposon present on pDonor is integrated into the pTarget plasmid, downstream of the 32-bp target site complementary to the gRNA sequence. TnsABf refers to an engineered fusion protein in which the polypeptide sequences for TnsA and InsB are fused and connected with a linker sequence that also encodes a nuclear localization signal. Isolated DNA may be tested directly for the presence of integrated pTarget product, based on unique and characteristic junction PCR products specific to the expected transposition product. In control experiments, the gRNA sequence was replaced with a non-targeting (scrambled) control; and/or the pTarget plasmid may also be modified to eliminate the target site; and/or one or more expression vectors may be omitted from the transfection mix; and/or one or more expression vectors may contain point mutations in the amino acid sequence of a necessary protein that will lead to an inability for the CRISPR-Tn system to enzymatically perform transposition.

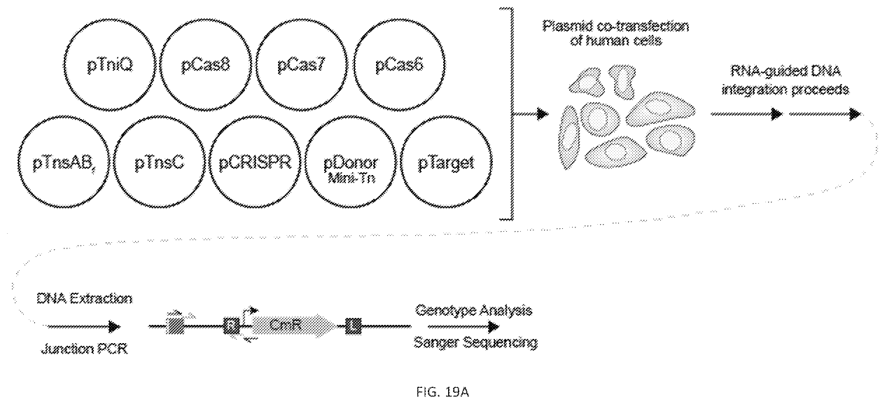

To assess RNA-guided DNA transposition activity in human cells, HEK293T cells were transfected with plasmid mixtures using Lipofectamine 2000 and standard protocols. Plasmid sequences are described in Table 8, and plasmid combinations used in transfections are described in Table 9. Cells were cultured at 37° C. with 5% CO₂, the media was replaced approximately 24 hours after transfection, and cells were harvested for analysis 72 hours post-transfection. DNA was harvested from HEK293T cells using QuickExtract DNA Extraction Solution (Lucigen) and standard protocols. Various PCR reactions were then performed on genomic lysates. In order to increase the sensitivity of the PCR reactions, nested PCR in which a small aliquot of a completed PCR reaction is carried over to a new PCR reaction in which new primers are used that anneal within the expected amplicon from the original PCR may be used. FIG. 19 describes the associated workflow to detect RNA-guided DNA integration.

When all requisite expression vectors, a gRNA expression vector that targets the same DNA sequence as used for TnsC-based transcriptional activation, and both pDonor and pTarget were co-transfected, evidence of RNA-guided transposition with Tn7016 based on the presence of junction amplicons via nested PCR was obtained. These amplicons were not produced when a gRNA expression vector was used that encoded a non-targeting (scrambled) sequence. When the amplicons from duplicate biological transfections were sequenced using a primer that anneals to the right end of the Tn7016 mini-Tn, the expected genotype was observed in which the primary product from the population contained the mini-Tn integrated 49-bp downstream of the target sequence matching the gRNA spacer.

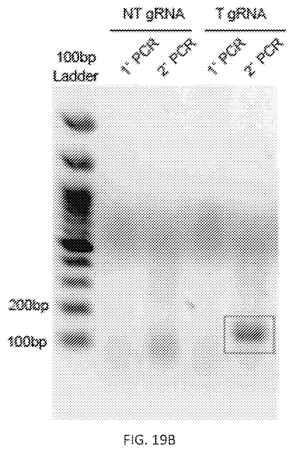

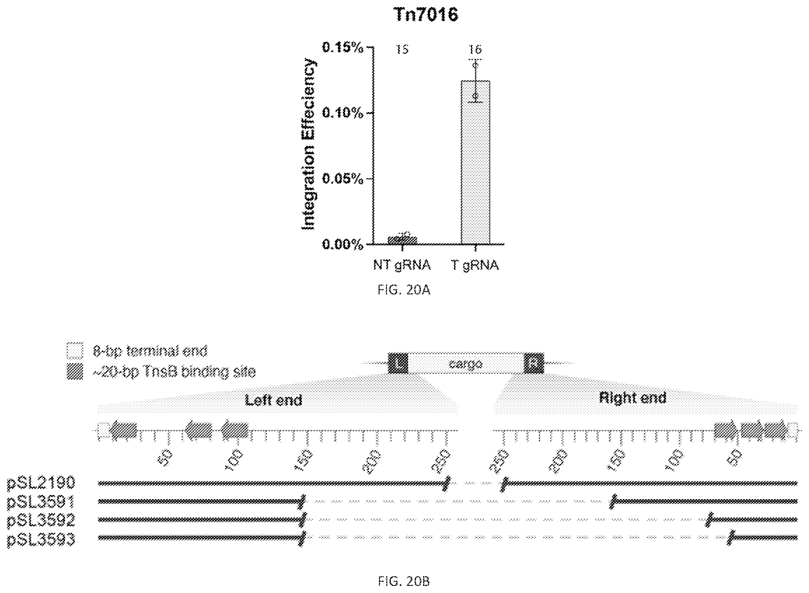

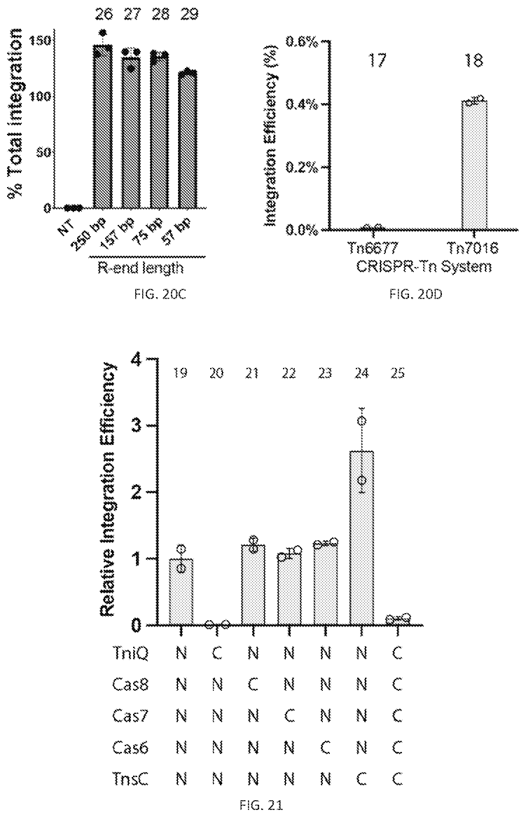

Primers and probes were designed to selectively amplify, and therefore quantify, insertion events via quantitative real-time PCR. By comparing the amplification of insertion events to the amplification of a region of the target plasmid that does not contain insertion events, an editing efficiency was estimated to range from 0.1-0.4% (FIGS. 20A and 20D), representing an approximately 50× increase relative to the system from Tn6677 tested under similar conditions. This value also represents a lower estimate since there was no selection for transfected cells in these experiments.

In order to streamline the donor DNA construct, the transposon ends of Tn7016 were rationally truncated, as was previously done with Tn6677 (Klompe et al., Nature 571, 219-225 (2019)). These designs were tested in both bacterial cells and human cells for RNA-guided DNA integration activity. Starting pDonor designs contained 250-bp derived from the E. ascidiicola genome at both transposon ends, despite knowledge from prior work that these sequences encompass both the minimal transposon ends as well as additional transposon sequence that is not important for transposase-transposon DNA recognition. During rational engineering of the transposon ends, the left end was truncated to a length of 145 base pairs (bp), counting from the terminal 5′-TG directly at the genome-transposon junction), and the right end was truncated to lengths of either 157 bp, 75 bp, or 57 bp (FIG. 20B) Relative to the starting pDonor that contained 250-bp at both ends, the truncated variants were equivalently active in E. coli for RNA-guided DNA integration (FIG. 20C).

Using the same truncated pDonor designs, but with vectors used for RNA-guided DNA integration in human cells, integration events were genotyped using the primers to amplify both Tn6677 and Tn7016 integration products for quantitative real-time PCR analysis. Biological duplicate integration assays were performed in which either Tn6677 or Tn7016 mobilized their respective mini-Tn substrates on pDonor to pTarget using the exact same 32-nt gRNA spacer sequence. Quantitative PCR analysis revealed that Tn7016 exhibited approximately 50× higher integration efficiency compared to Tn6677 (FIG. 20D), with the truncated transposon end pDonor construct.

Tn7016 components may exhibit optimal performance with NLS tag placement that is distinct from the optimal placement observed with components from Tn6677. Previous integration assays using Tn7016 protein components contained an N-terminal NLS tag, except for TnsAB_{f}, which contained an internal NLS tag at the junction of TnsA and TnsB. Whether relocation of the NLS tag to the C-terminus of certain proteins would increase the overall integration efficiency was tested. In order to investigate potential tolerance towards C-terminal NLS tags, NLS tags were individually relocated from the N-terminus to the C-terminus in each component, and then its impact on transposition efficiency while all other protein components maintained N-terminal NLS tags was analyzed. As shown in FIG. 21, Tn7016 is notably tolerant to various C-terminal NLS placements, wherein migrating the NLS tag to the C-terminal end of Cas8, Cas7, and Cas6 showed no drop in integration efficiency relative to the condition in which all N-terminal termini were tagged. Additionally, these experiments demonstrated that switching the NLS tag from the N-terminus to the C-terminus of TnsC resulted in a marked increase in integration efficiency. This demonstrates that protein components from Tn7016 show unique preference/allowance for terminal tagging.

Proteins which show permissiveness towards C-terminal tagging may be tagged with additional epitope tags, and/or “ribosomal skipping” 2A peptides. In certain embodiments, the inclusion of C-terminal 2A peptide tags enabled the construction of polycistronic expression vectors, wherein multiple protein components are encoded on a single fusion mRNA transcript but translated as distinct polypeptides. This allowed reduction in the total number of individual plasmids that need to be delivered for expression of all the necessary components. In embodiments where mRNA is delivered directly to cells, in lieu of plasmid DNA, the same strategy enabled delivery of fewer distinct mRNA molecules. For example, rather than delivery Cas6, Cas7, and Cas8 mRNA separately, a mRNA encoding Cas6-2A-Cas7-2A-Cas8 could be delivered, whereby the 2A sequence leads to termination and translation initiation in cells, such that individual Cas6, Cas7, and Cas8 polypeptides are generated.

TABLE 8
Sequence and description of plasmids used in RNA-guided DNA
targeting and/or integration experiments in eukaryotes

| Plasmid ID | Plasmid name |
|---|---|
| pSL0341 | pTarget(mCherry reporter for CRISPRa and pTarget) |
| pSL1409 | pCRISPR-NT [p6A_Vch_hU6_CRISPR(tSL0105)] |
| pSL2084 | pCRISPR_T [p6A_Vch_hU6_CRISPR(tSL0264)] |
| pSL2123 | Tn6677_pDonor (pR6K_Vch_TnR(57bp)_Pcat_CmR_ToL) |
| pSL2190 | Tn7016_pDonor(pUC57_pDonor_TnR(250bp)_TnL(250bp)) |
| pSL2620 | pTniQ (pcDNA3.1_hCO_Vch_BP-NLS-TniQ) |
| pSL2621 | pCas8 (pcDNA3.1_hCO_Vch_BP-NLS-Cas8) |
| pSL2622 | pCas7 (pcDNA3.1_hCO_Vch_BP-NLS-Cas7) |
| pSL2623 | pCas6 (pcDNA3.1_hCO_Vch_BP-NLS-Cas6) |
| pSL2645 | pTnsC (pcDNA3.1_hCO_Vch_BP-NLS-TnsC) |
| pSL2669 | pTnsABf (pcDNA3.1_hCO_Vch_TnsA_BP-NLS_TnsB) |
| pSL2783 | p6A_hCO_Vch_TnsC_BP-NLS-VP64 |
| pSL2880 | pThiQ (pcDNA3.1_BP-NLS_ThiQ Tn7011 (Homolog 3)) |
| pSL2881 | pCas8 (pcDNA3.1_BP-NLS_Cas8 Tn7011 (Homolog 3)) |
| pSL2882 | pCas7 (pcDNA3.1_BP-NLS_Cas7 Tn7011 (Homolog 3)) |
| pSL2883 | pCas6 (pcDNA3.1_BP-NLS_Cas6 Tn7011 (Homolog 3)) |
| pSL2884 | pTnsC (pcDNA3.1_BP-NLS_TnsC Tn7011 (Homolog 3)) |
| pSL2885 | p6A_Tn7011_hU6_CRISPR_NT |
| pSL2886 | p6A_Tn7011_hU6_CRISPR_tSL0264 |
| pSL2887 | TnsC-VP64_Tn7011 |
| pSL2888 | pTniQ (pcDNA3.1_BP-NLS_TniQ Tn7010 (Homolog 5)) |
| pSL2889 | pCas8 (pcDNA3.1_BP-NLS_Cas8 Tn7010 (Homolog 5)) |
| pSL2890 | pCas7 (pcDNA3.1_BP-NLS_Cas7 Tn7010 (Homolog 5)) |
| pSL2891 | pCas6 (pcDNA3.1_BP-NLS_Cas6 Tn7010 (Homolog 5)) |
| pSL2892 | pTnsC (pcDNA3.1_BP-NLS_TnsC Tn7010 (Homolog 5)) |
| pSL2893 | p6A_Tn7010_hU6_CRISPR_NT |
| pSL2894 | p6A_Tn7010_hU6_CRISPR_tSL0264 |
| pSL2895 | TnsC-VP64_Tn7010 |
| pSL2896 | pTniQ (pcDNA3.1_BP-NLS_TniQ Tn7015 (Homolog 6)) |
| pSL2897 | pCas8 (pcDNA3.1_BP-NLS_Cas8 Tn7015 (Homolog 6)) |
| pSL2898 | pCas7 (pcDNA3.1_BP-NLS_Cas7 Tn7015 (Homolog 6)) |
| pSL2899 | pCas6 (pcDNA3.1_BP-NLS_Cas6 Tn7015 (Homolog 6)) |
| pSL2900 | pTnsC (pcDNA3.1_BP-NLS_TnsC Tn7015 (Homolog 6)) |
| pSL2901 | p6A_Tn7015_hU6_CRISPR_NT |
| pSL2902 | p6A_Tn7015_hU6_CRISPR_tSL0264 |
| pSL2903 | TnsC-VP64_Tn7015 |
| pSL2904 | pTniQ (pcDNA3.1_BP-NLS_TniQ Tn7005 (Homolog 12)) |
| pSL2905 | pCas8 (pcDNA3.1_BP-NLS_Cas8 Tn7005 (Homolog 12)) |
| pSL2906 | pCas7 (pcDNA3.1_BP-NLS_Cas7 Tn7005 (Homolog 12)) |
| pSL2907 | pCas6 (pcDNA3.1_BP-NLS_Cas6 Tn7005 (Homolog 12)) |
| pSL2908 | pTnsC (pcDNA3.1_BP-NLS_TnsC Tn7005 (Homolog 12)) |
| pSL2909 | p6A_Ta7005_hU6_CRISPR_NT |
| pSL2910 | p6A_Tn7005_hU6_CRISPR _tSL0264 |
| pSL2911 | TnsC-VP64_Tn7005 |
| pSL2912 | pTniQ (pcDNA3.1_BP-NLS_ThiQ Tn7016 (Homolog 17)) |
| pSL2913 | pCas8 (pcDNA3.1_BP-NLS_Cas8 Tn7016 (Homolog 17)) |
| pSL2914 | pCas7 (pcDNA3.1_BP-NLS_Cas7 Tn7016 (Homolog 17)) |
| pSL2915 | pCas6 (pcDNA3.1_BP-NLS_Cas6 Tn7016 (Homolog 17)) |
| pSL2916 | pTnsC (pcDNA3.1_BP-NLS_TnsC Tn7016 (Homolog 17)) |
| pSL2917 | p6A_Tn7016_hU6_CRISPR_NT |
| pSL2918 | p6A_Tn7016_hU6_CRISPR_tSL0264 |
| pSL2919 | TnsC-VP64_Tn7016 |
| pSL2920 | pTniQ (pcDNA3.1_BP-NLS_TniQ Tn7003 (Homolog 18)) |
| pSL2921 | pCas8 (pcDNA3.1_BP-NLS_Cas8 Tn7003 (Homolog 18)) |
| pSL2922 | pCas7 (pcDNA3.1_BP-NLS_Cas7 Tn7003 (Homolog 18)) |
| pSL2923 | pCas6 (pcDNA3.1_BP-NLS_Cas6 Tn7003 (Homolog 18)) |
| pSL2924 | pTnsC (pcDNA3.1_BP-NLS_TnsC Tn7003 (Homolog 18)) |
| pSL2925 | p6A_Tn7003_hU6_CRISPR_NT |
| pSL2926 | p6A_Tn7003_hU6_CRISPR_tSL0264 |
| pSL2927 | TnsC-VP64_Tn7003 |
| pSL3402 | pTnsAB(pcDNA3.1_TnsA _BP-NLS_TnsB Tn7016 (Homolog 17)) |
| pSL3430 | Tn7016_pDonor (pR6K_Tn7016_TnR_Pcat_CmR_TnL) |
| pSL3628 | pTniQ (pcDNA3.1_hCO_Tn7016_TniQ-BP-NLS) |
| pSL3629 | pCas8 (pcDNA3.1_hCO_Tn7016_Cas8-BP-NLS) |
| pSL3630 | pCas7 (pcDNA3.1_hCO_Tn7016_Cas7-BP-NLS) |
| pSL3631 | pCas6 (pcDNA3.1_hCO_Tn7016_Cas6-BP-NLS) |
| pSL3632 | pTnsC (pcDNA3.1_hCO_Tn7016_TnsC-NLS-BP-NLS) |
| pSL3591 | pUC57_pDonor_I-Fv_Pseudoalteromonas_sp.S983(Tn7016)_RE-157bp_LE-145bp |
| pSL3592 | pUC57_pDonor_I-Fv_Pseudoalteromonas_sp.S983(Tn7016)_RE-75bp_LE-145bp |
| pSL3593 | pUC57_pDonor_I-Fv_Pseudoalteromonas_sp.S983(Tn7016)_RE-57bp_LE-145bp |
| pSL2158 | pCDF_pCQT(tSL0004)_I-Fv_Pseudoalteromonas_sp.S983(Tn7016) |

TABLE 9
Table of plasmids used in transformation and/or transfection experiments

| Transfection | |
|---|---|
| ID # | Plasmids used in experiment |
| 1 | pSL0341, pSL2084, pSL2620, pSL2621, pSL2622, pSL2623, pSL2783 |
| 2 | pSL0341, pSL1409, pSL2620, pSL2621, pSL2622, pSL2623, pSL2783 |
| 3 | pSL0341, pSL2920, pSL2921, pSL2922, pSL2923, pSL2927, pSL2926 |
| 4 | pSL0341, pSL2920, pSL2921, pSL2922, pSL2923, pSL2927, pSL2925 |
| 5 | pSL0341, pSL2904, pSL2905, pSL2906, pSL2907, pSL2911, pSL2910 |
| 6 | pSL0341, pSL2904, pSL2905, pSL2906, pSL2907, pSL2911, pSL2909 |
| 7 | pSL0341, pSL2888, pSL2889, pSL2890, pSL2891, pSL2895, pSL2894 |
| 8 | pSL0341, pSL2888, pSL2889, pSL2890, pSL2891, pSL2895, pSL2893 |
| 9 | pSL0341, pSL2880, pSL2881, pSL2882, pSL2883, pSL2887, pSL2886 |
| 10 | pSL0341, pSL2880, pSL2881, pSL2882, pSL2883, pSL2887, pSL2885 |
| 11 | pSL0341, pSL2896, pSL2897, pSL2898, pSL2899, pSL2903, pSL2902 |
| 12 | pSL0341, pSL2896, pSL2897, pSL2898, pSL2899, pSL2903, pSL2901 |
| 13 | pSL0341, pSL2912, pSL2913, pSL2914, pSL2915, pSL2919, pSL2918 |
| 14 | pSL0341, pSL2912, pSL2913, pSL2914, pSL2915, pSL2919, pSL2917 |
| 15 | pSL0341, pSL2912, pSL2913, pSL2914, pSL2915, pSL2916, pSL2917, pSL3402, pSL3430 |
| 16 | pSL0341, pSL2912, pSL2913, pSL2914, pSL2915, pSL2916, pSL2918, pSL3402, pSL3430 |
| 17 | pSL0341, pSL2084, pSL2123, pSL2620, pSL2621, pSL2622, pSL2623, pSL2645, pSL2669 |
| 18 | pSL0341, pSL2912, pSL2913, pSL2914, pSL2915, pSL2916, pSL2918, pSL3402, pSL3593 |
| 19 | pSL0341, pSL2912, pSL2913, pSL2914, pSL2915, pSL2916, pSL2918, pSL3402, pSL3593 |
| 20 | pSL0341, pSL3628, pSL2913, pSL2914, pSL2915, pSL2916, pSL2918, pSL3402, pSL3593 |
| 21 | pSL0341, pSL2912, pSL3629, pSL2914, pSL2915, pSL2916, pSL2918, pSL3402, pSL3593 |
| 22 | pSL0341, pSL2912, pSL2913, pSL3630, pSL2915, pSL2916, pSL2918, pSL3402, pSL3593 |
| 23 | pSL0341, pSL2912, pSL2913, pSL2914, pSL3631, pSL2916, pSL2918, pSL3402, pSL3593 |
| 24 | pSL0341, pSL2912, pSL2913, pSL2914, pSL2915, pSL3632, pSL2918, pSL3402, pSL3593 |
| 25 | pSL0341, pSL3628, pSL3629, pSL3630, pSL3631, pSL3632, pSL2918, pSL3402, pSL3593 |
| 26 | pSL2158, pSL2190 |
| 27 | pSL2158, pSL3591 |
| 28 | pSL2158, pSL3592 |
| 29 | pSL2158, pSL3593 |

### Example 13

### RNA-Guided DNA Integration in Human Cells

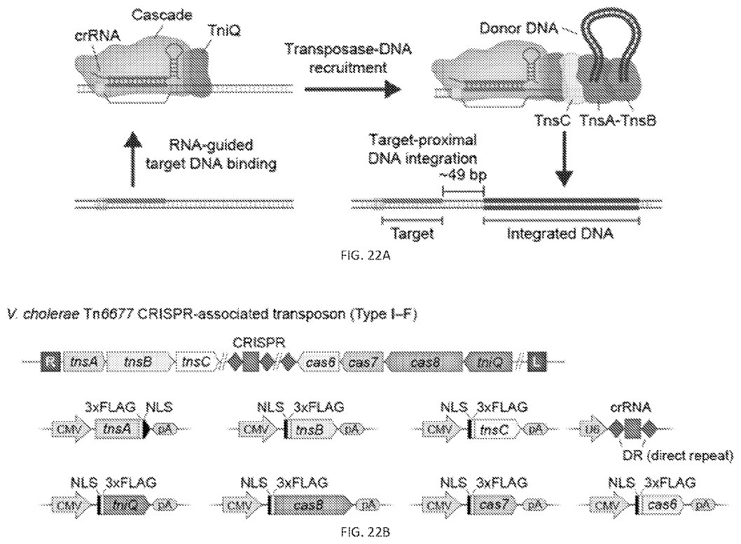

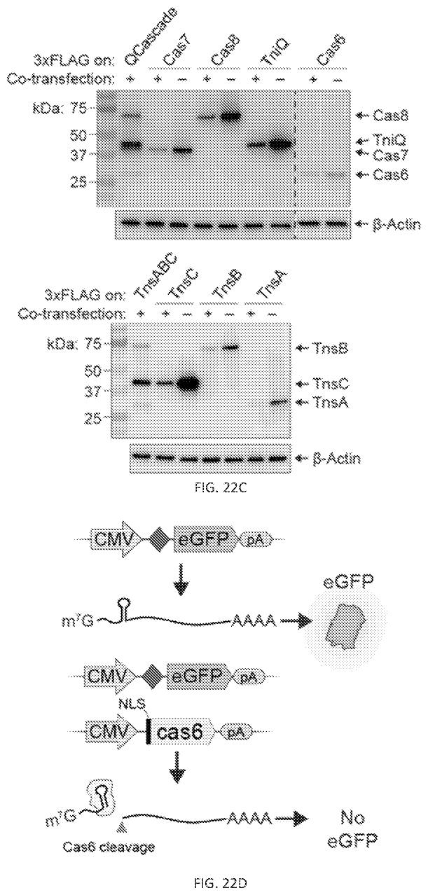

Using a Type I-F system derived from Vibrio cholerae Tn6677, DNA insertions were demonstrated in multiple bacterial species that exhibited exquisite genome-wide specificity and could be easily reprogrammed to user-defined sites with single-bp accuracy. Long-read whole-genome sequencing confirmed the purity of integration products, and additional heterologous reconstitution experiments demonstrated autonomous enzymatic function independent of obligate recombination factors. RNA-guided transposases were leveraged for targeted DNA integration in mammalian cells, despite the formidable obstacle of reconstituting a complex, multi-component pathway that depends on a donor DNA, guide CRISPR RNA (crRNA), and assembly of seven distinct proteins, many of which function in an oligomeric state (FIGS. 22A and 22B).

Bacterial Tn7-like transposons have co-opted at least three distinct types of nuclease-deficient CRISPR-Cas systems for RNA-guided transposition (I—B, I—F, and V-K), with each exhibiting unique features. Fidelity and programmability parameters for experimentally characterized CRISPR-transposon systems, alongside recently described Cas9-transposase fusion approaches, were carefully reviewed. Type I-F V. cholerae CRISPR-associated transposon (VchINTEGRATE, or VchINT) was of particular focus because of its optimal integration efficiency, specificity, and absence of cointegrates. Within this system, a ribonucleoprotein complex comprising TniQ and Cascade (VchQCascade, with stoichiometry Cas8₁-Cas7₆-Cas6₁-crRNA₁-TniQ₂) performs RNA-guided DNA targeting, thereby defining sites for transposon DNA insertion. Excision and integration reactions are catalyzed by the heteromeric TnsA-TnsB transposase, but only after prior recruitment of the AAA+ ATPase, TnsC. Although the stoichiometry of TnsABC in the final holo-transpososome is not known, ˜6 copies of a InsAB heterodimer and 7 or more copies of TnsC are likely optimal.

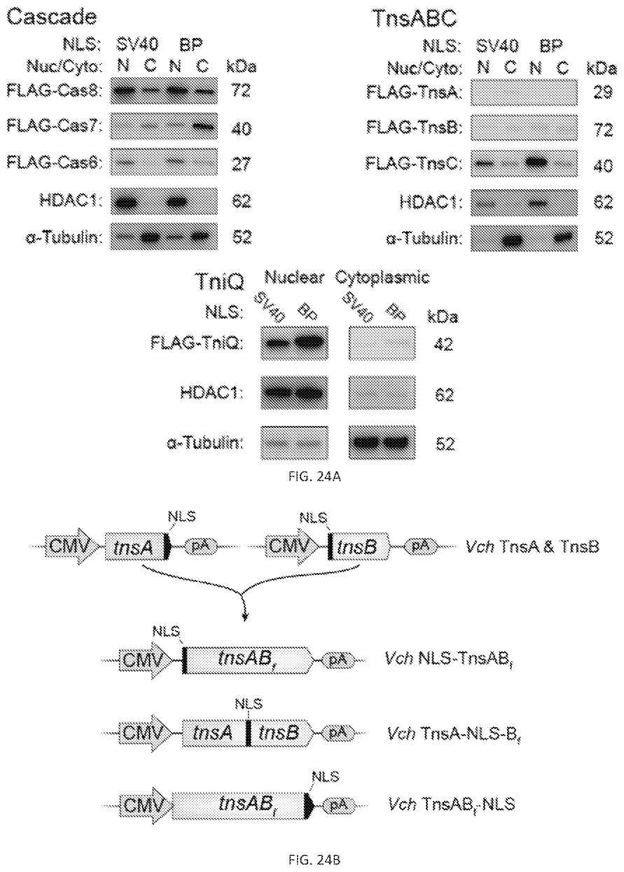

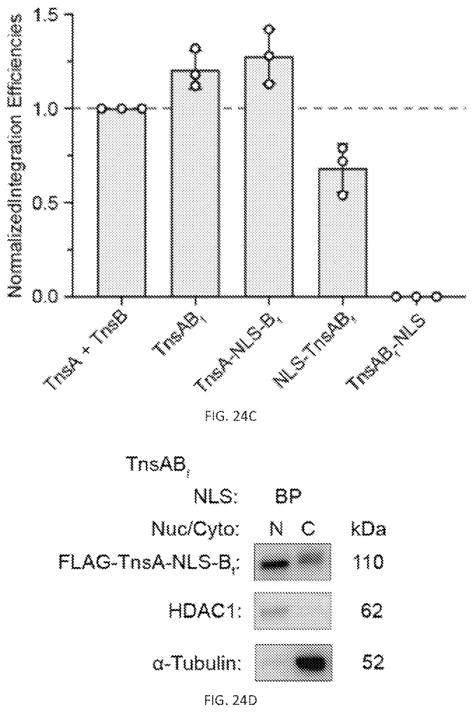

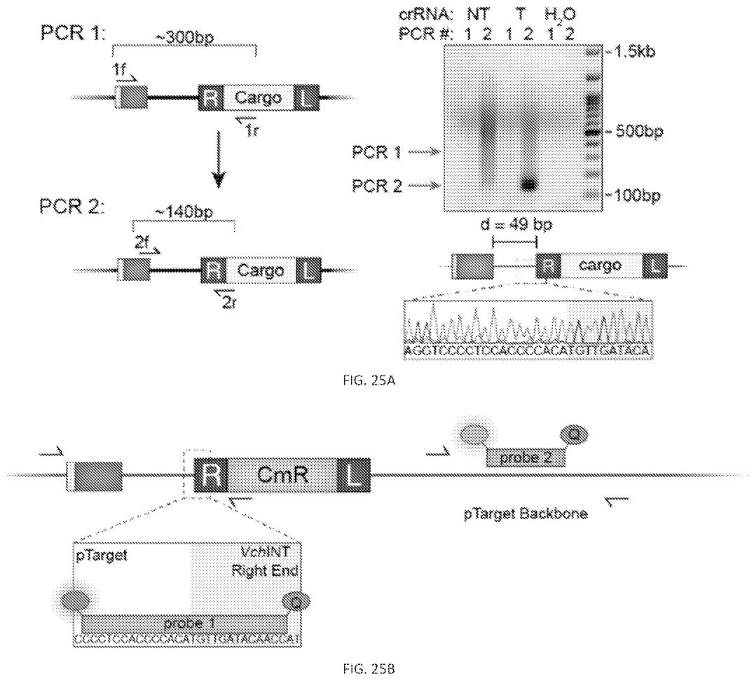

A methodical, bottom-up approach was adopted to port VchINT into human cells. Whether the component parts were being efficiently expressed, each protein-coding gene was cloned onto a standard mammalian expression vector with an N- or C-terminal nuclear localization signal (NLS) and 3×FLAG epitope tag (FIG. 22B). Using Western blotting, robust heterologous protein expression, both individually and when all INTEGRATE proteins were co-expressed, was observed (FIG. 22C). Cellular fractionation provided evidence of nuclear trafficking, and efficient expression and trafficking of an engineered TnsAB fusion protein (TnsAB_{f}) that was previously shown to retain wild-type activity was also demonstrated (FIG. 24).

To assess guide RNA expression, a previously developed approach to monitor crRNA biogenesis within the 5′ untranslated region (UTR) of a messenger RNA encoding GFP was adapted. Cas6 is a ribonuclease subunit of Cascade that cleaves the CRISPR repeat sequence in most Type I CRISPR-Cas systems, which in the assay would sever the 5′ cap from the GFP open reading frame and thus lead to fluorescence knockdown (FIG. 22D). A near-total loss of GFP fluorescence was observed when the reporter plasmid was co-transfected with cognate VchCas6, but not when the reporter encoded a non-cognate CRISPR repeat or lacked a repeat altogether (FIG. 22E). Interestingly, GFP knockdown was substantially reduced when Cas6 contained a C-terminal NLS or 2A peptide (FIG. 22E), indicating a sensitivity to terminal tagging that could not be easily explained by the cryoEM structure. Collectively, these experiments verified expression of all protein and RNA components from VchINT, leading us to next focus on functional reconstitution of RNA-guided DNA targeting by QCascade.

A promoter-driven chloramphenicol resistance cassette (CmR) was cloned within the mini-transposon of a donor plasmid (pDonor), and the same sequence on the mCherry reporter plasmid (pTarget) that was used in transcriptional activation experiments was targeted. Upon successful transposition in HEK293T cells, integrated pTarget products will carry both CmR and KanR drug markers and can thus be selected for by transforming E. coli with plasmid DNA isolated from transfected cells (FIG. 14A). In these experiments a pDonor backbone that cannot be replicated in standard E. coli strains was used, reducing background from unreacted plasmids. A TnsAB fusion protein (InsABf) that contains an internal bipartite NLS and maintains wild-type activity in E. coli (FIG. 24C) was also used, thereby reducing the number of unique protein components.

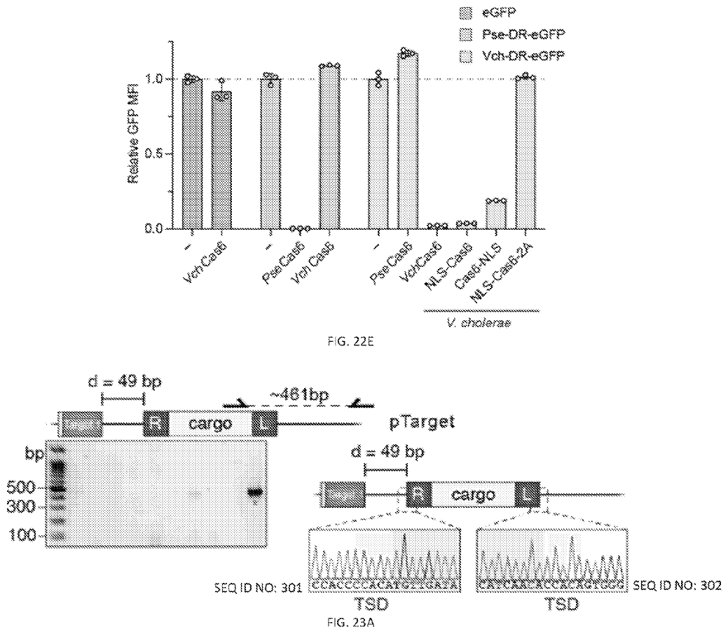

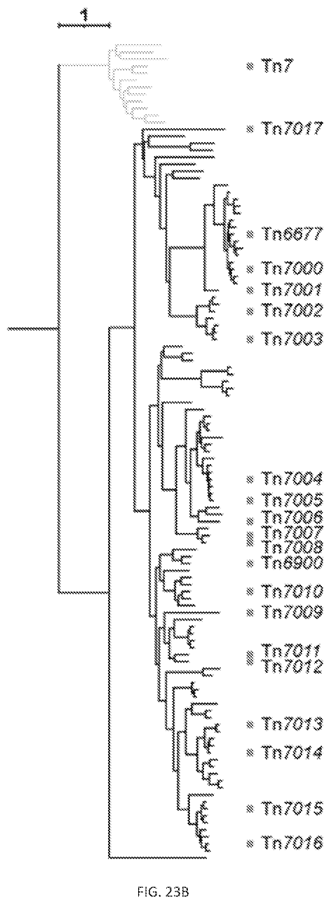

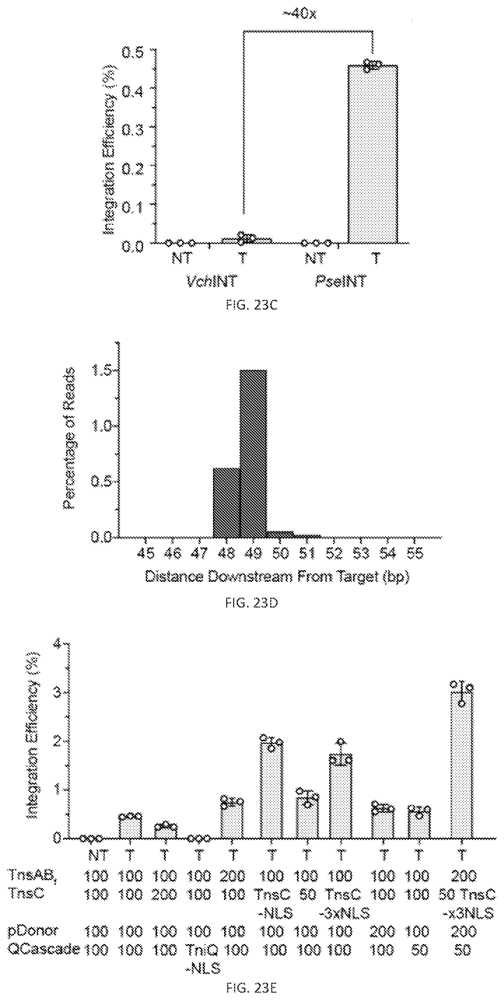

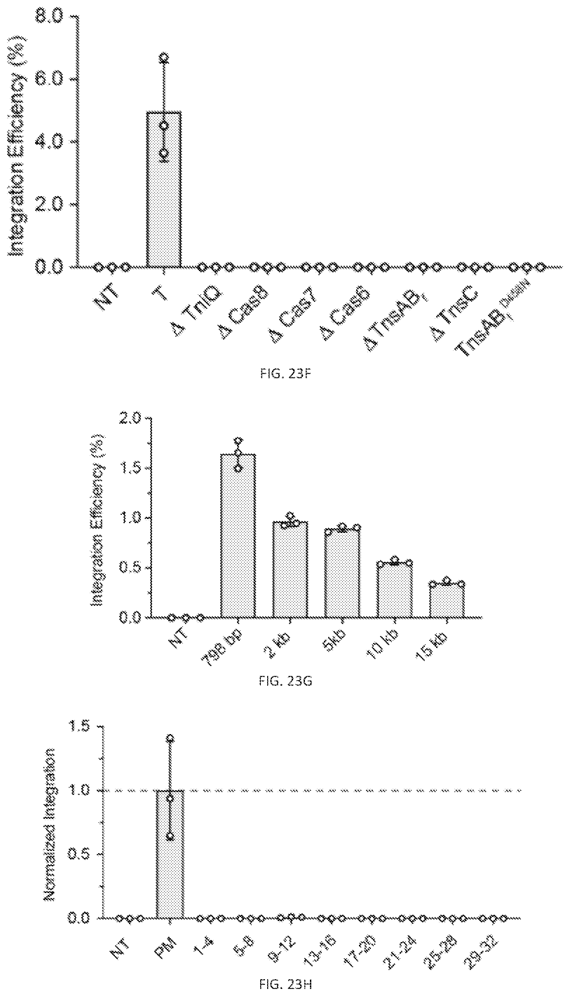

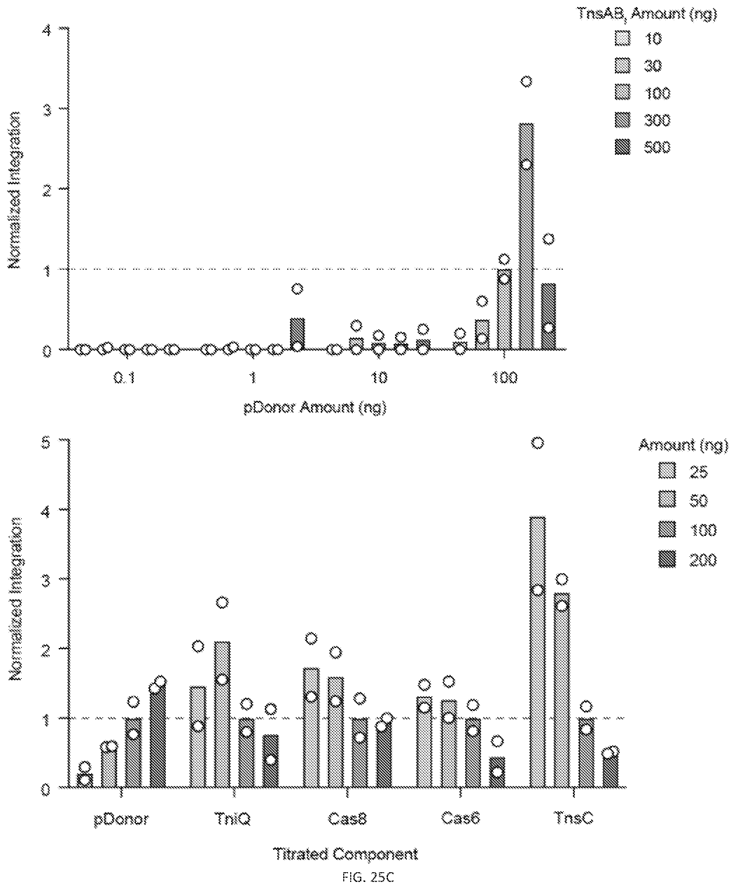

After transfecting HEK293T cells with pDonor, pTarget, and all protein-crRNA expression plasmids, purifying the plasmid mixture from cells, and using the mixture to transform E. coli, the emergence of colonies that were chloramphenicol and kanamycin resistant were observed, which outnumbered the corresponding colonies obtained in non-targeting control experiments. Junction PCR was performed on select colonies and bands of the expected size were obtained, which subsequent Sanger sequencing confirmed were integration products arising from DNA transposition 49-bp downstream of the target site (FIG. 23A). The same products were detected by nested PCR directly from HEK293T cell lysates (FIG. 25A), and a sensitive Taqman probe-based qPCR strategy was developed to quantify integration events from lysates by detecting site-specific, plasmid-transposon junctions (FIG. 25B). Using this approach, an initial optimization screen was performed by varying the relative amounts of expression and pDonor plasmids and efficiencies were greatest with low levels of pTnsC and high levels of pTnsAB_{f} and pDonor (FIG. 25C) Absolute efficiencies of plasmid-to-plasmid transposition were <1%.

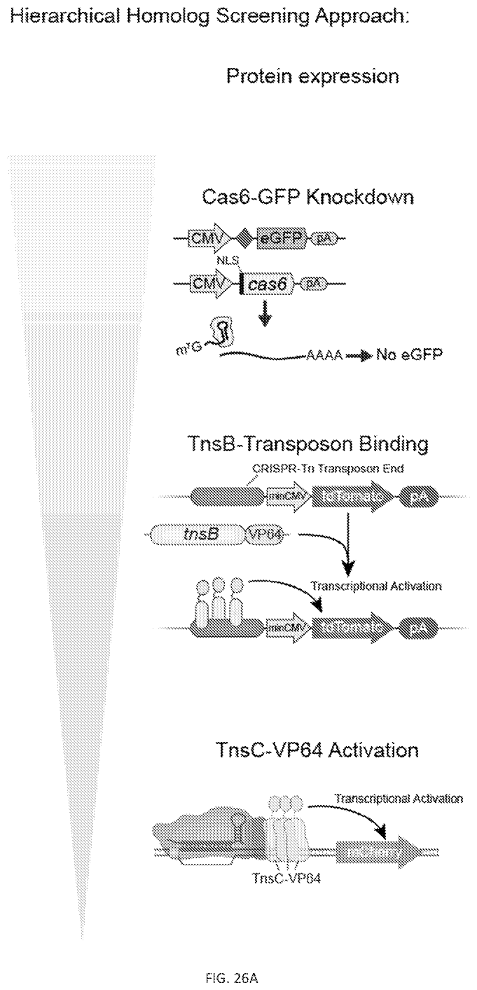

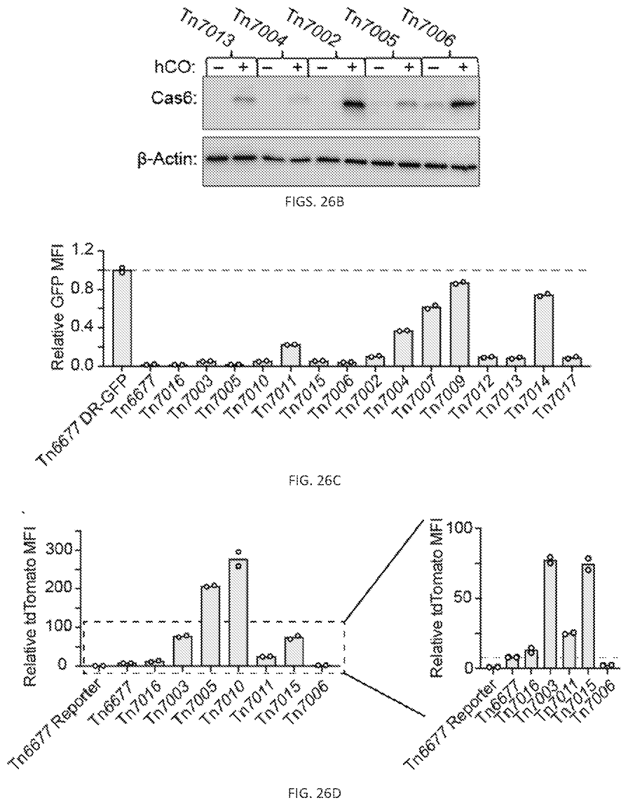

Bioinformatic mining and experimental characterization identified 18 new Type I-F3 CRISPR-associated transposons (Tn7000-Tn7017), many of which exhibit high-efficiency and high-fidelity RNA-guided DNA integration in E. coli. A hierarchical screening approach was used to uncover variants with improved activity in human cells (FIG. 26A). Briefly, the screening approach involved filtering based on robust activity in three key areas: (i) crRNA biogenesis by Cas6, assessed using the GFP knockdown assay; (ii) transposon DNA binding by TnsB, assessed using a tdTomato reporter assay; and (iii) transcriptional activation by TnsC-VP64, assessed using the mCherry reporter assay. In all cases, genes were human codon optimized, which often facilitated strong expression (FIG. 26B), and tagged with NLS sequences on the same termini as for Tn6677 (VchINT). The majority of systems exhibited efficient crRNA biogenesis and transposon DNA binding activity that was similar to that observed with Tn6677 (FIGS. 26C and 26D). Tn7016 showed reproducible induction of mCherry expression, albeit at levels ˜8-fold lower than Tn6677 (FIG. 26E). Tn7016, a 31-kb transposon from Pseudoalteromonas sp. S983, hereafter PseINT, was investigated for its RNA-guided DNA integration activity.

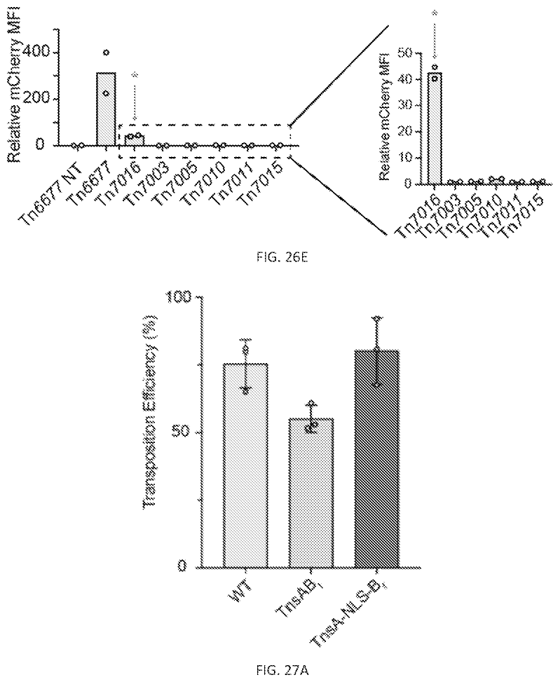

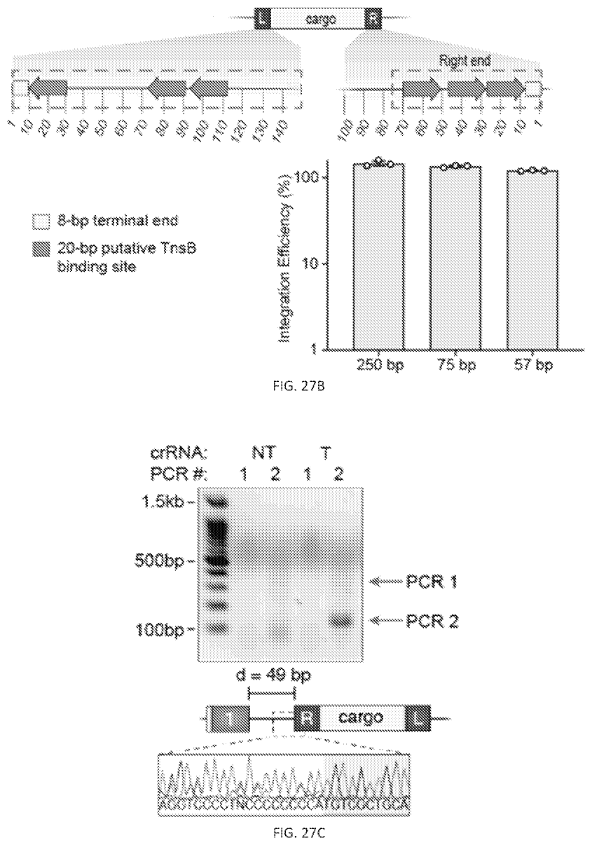

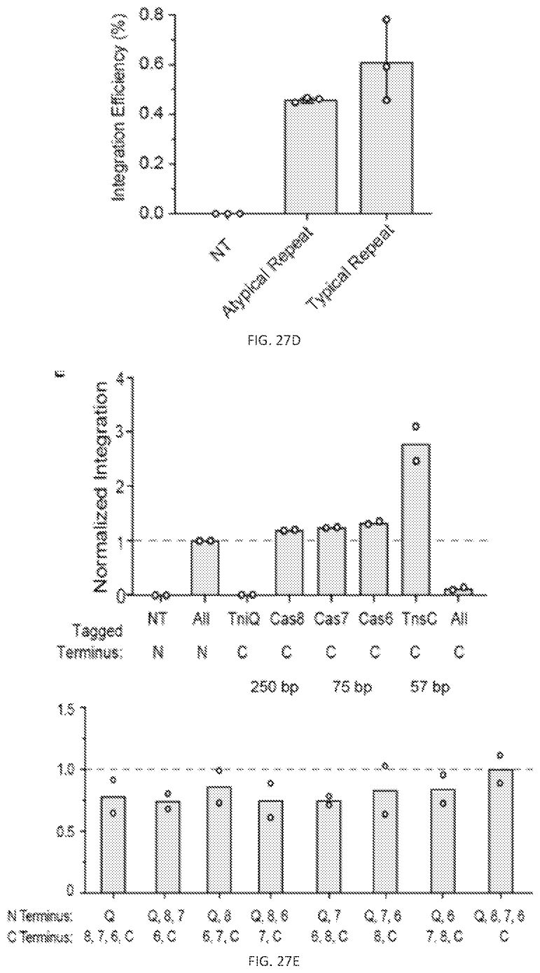

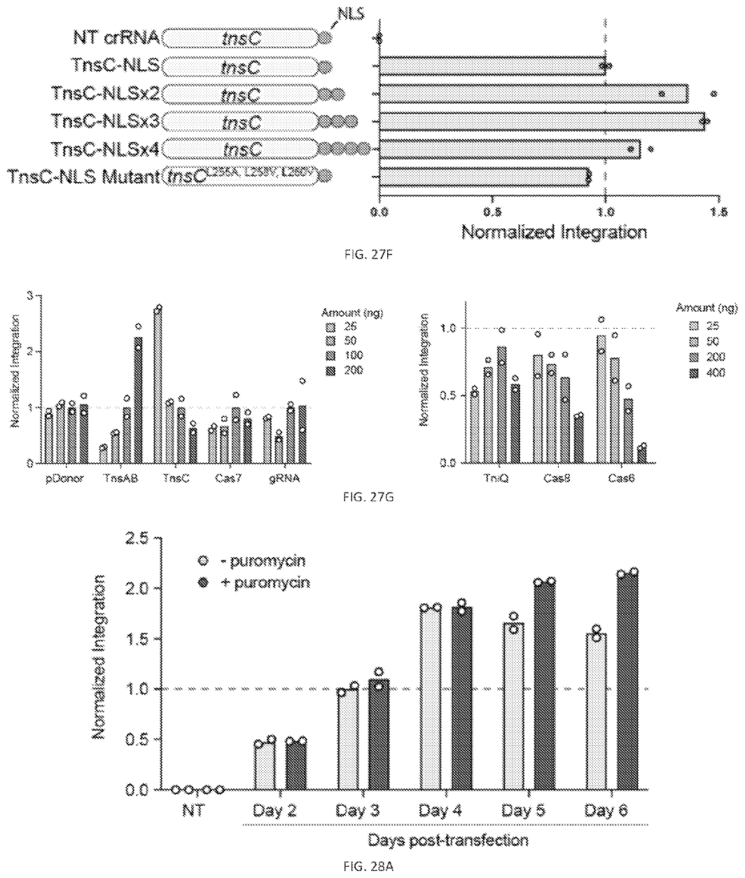

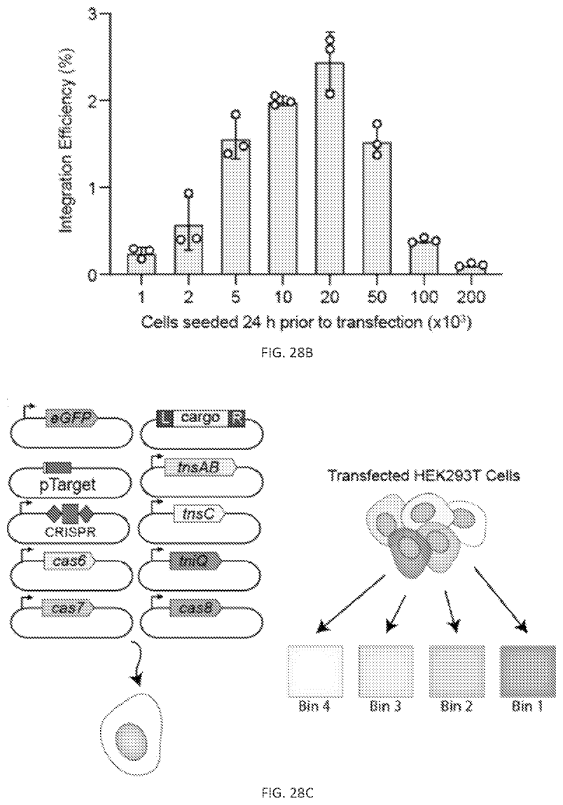

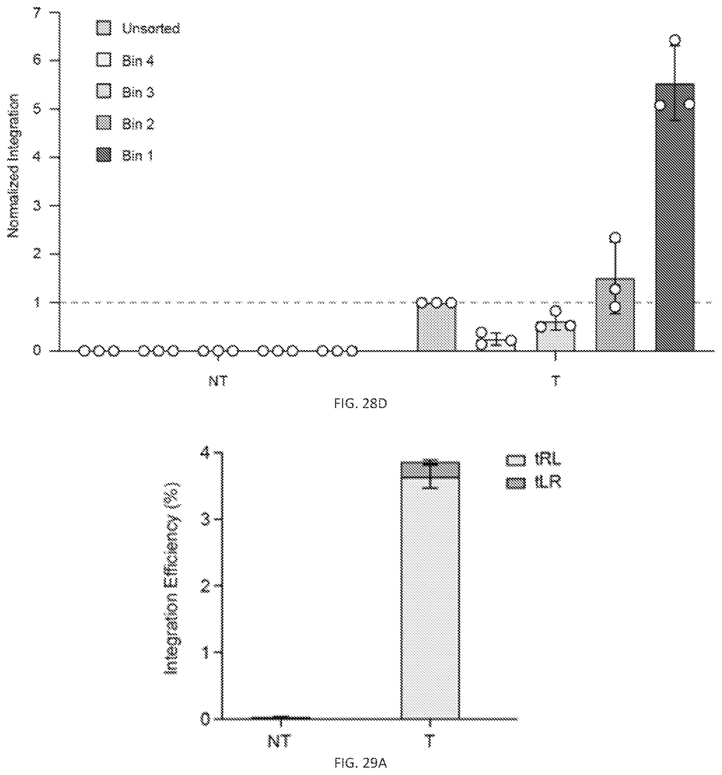

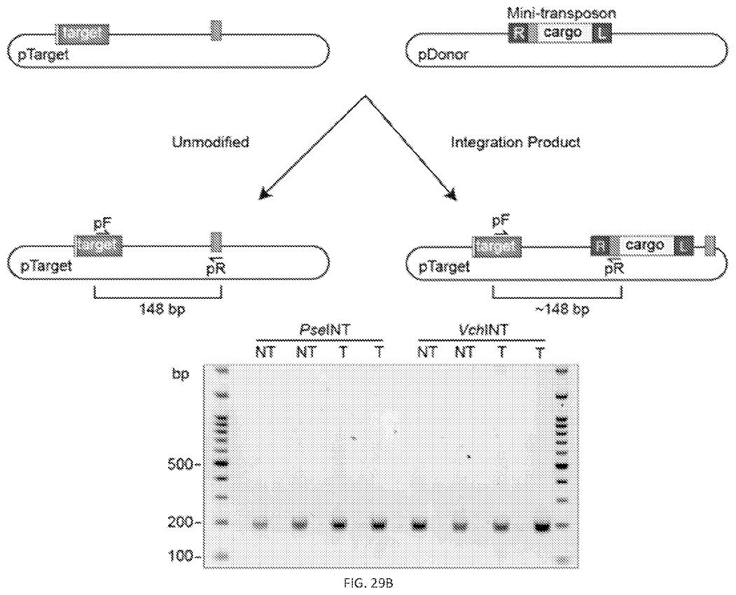

After verifying that fusing TnsA and TnsB from PseINT with an internal NLS retained function, and optimizing the length of left and right transposon ends (FIGS. 27A and 27B), plasmid-to-plasmid transposition assays were repeated in HEK293T cells. PseINT was ˜40-fold more active than the most optimized version of VchINT when tested under unoptimized conditions, and PCR followed by Sanger or Illumina sequencing analysis confirmed the expected site of integration 49-bp downstream of the target (FIGS. 23C, 23D, and 27C). To further improve integration efficiencies, the design of the crRNA, location of NLS tags, and relative amounts of each expression plasmid, were systematically varied which collectively yielded a further ˜10-fold improvement to reach levels of 3-5% integration (FIGS. 23E and 27, FIGS. 27D-27G). In the course of these experiments, peak integration occurred 4-6 days post-transfection, and the integration efficiency was sensitive to cell density (FIGS. 28A and 28B). Since the experimental approach thus far involved co-transfection of nine distinct plasmids, that activity could vary considerably based on not only the stoichiometry of the transfected plasmids but also the range of plasmid amounts received across the population of cells. To test this, a GFP transfection marker was co-transfected and the top 20% brightest cells were into four bins based on their fluorescence level and then separately analyzed for integration. The integration efficiency increased concomitantly with GFP expression, with the top bin exhibiting >5-fold higher activity than the unsorted cell population (FIGS. 28C and 28D).

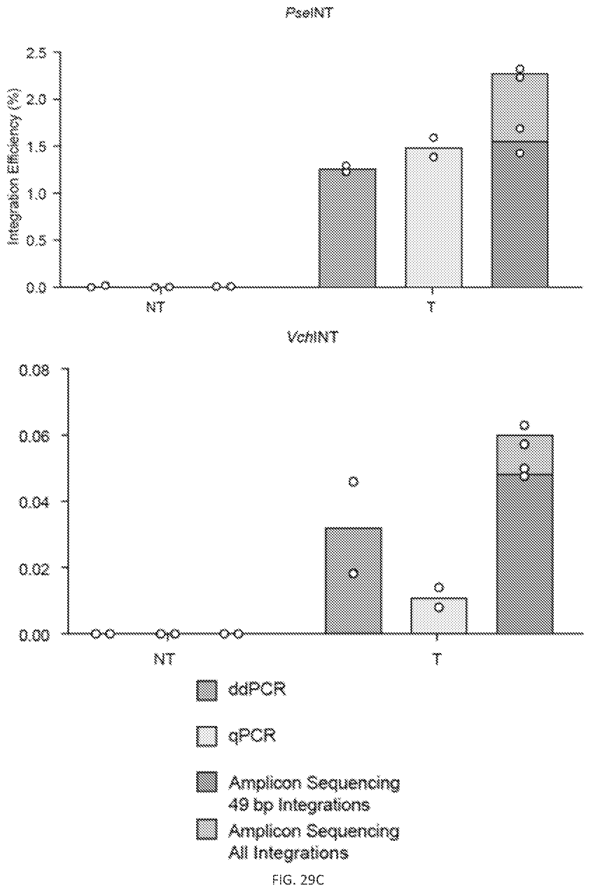

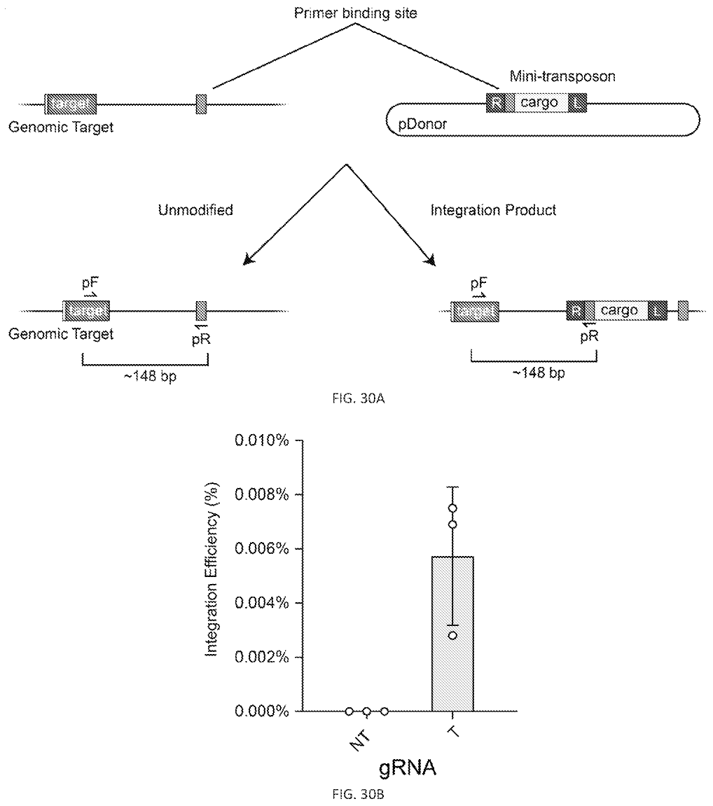

Transposition was conditional on a targeting crRNA and the presence of all protein components, including an intact TusB active site (FIG. 23F), and functioned with genetic payloads spanning 1-15 kb in size, albeit with a ˜3-fold decrease in efficiency with larger payloads (FIG. 23G). A panel of mismatched crRNAs was generated in which mutations were tiled along the length of the 32-nt guide, and activity was found to be ablated regardless of the location (FIG. 23H), indicating a greater degree of discrimination than that observed in activation experiments or in E. coli. Finally, an alternative qPCR approach was used to confirm that integration orientation for PseINT was highly biased towards tRL, and both droplet digital PCR (ddPCR) and amplicon sequencing were performed to further corroborate the quantitative data obtained from Taqman qPCR (FIG. 29).

TABLE 10

| Plasmid ID | Plasmid name |
|---|---|
| pSL0341 | mCherry reporter for CRISPRa |
| pSL0454 | pcDNA3.1 hCO pse_Cascade-Cas7-VP64 |
| pSL0532 | 6A U6-I-E_pse_CRISPR(Hsa07-2) |
| pSL0534 | 6A_hU6_I-E_PseS-6-2_CRISPR(non-targeting) |
| pSL2276 | Pse I-E_DR-eGFP |
| pSL2277 | Tn6677_DR-eGFP |
| pSL2279 | Pse I-E pCas6 |
| pSL812 | Vch stuffer crRNA |
| pSL2645 | Vch pTnsC |
| pSL3617 | Pse stuffer crRNA |
| pSL1567 | pCDF_Vch_PT7_CRISPR(Target4)_QCascade_TnsABC_T7Term w/all Permissive |
| | Eukaryotic Terminal Tags |
| pSL2912 | Pse pTniQ |
| pSL2913 | Pse pCas8 |
| pSL2914 | Pse pCas7 |
| pSL2915 | Pse pCas6 |
| pSL3713 | Pse pTnsC-3xNLS |
| pSL3402 | Pse pTnsA-NLS-Bf |
| pSL2620 | pcDNA3.1_hCO_Vch_BP-NLS-TniQ |
| pSL2621 | pcDNA3.1_hCO_Vch_BP-NLS-Cas8 |
| pSL2622 | pcDNA3.1_hCO_Vch_BP-NLS-Cas7 |
| pSL2623 | pcDNA3.1_hCO_Vch_BP-NLS-Cas6 |
| pSL3626 | Vch pDonor |
| pSL3637 | Pse pDonor |
| pSL2669 | pcDNA3.1_hCO_Vch_TnsA_BP-NLS_TnsB |
| pSL2693 | pcDNA3.1_hCO_VP64_Vch_BP-NLS-Cas7 |
| pSL2783 | p6A_hCO_Vch_TnsC_BP-NLS-VP64 |
| pSL1236 | pDonor |
| pSL1014 | pQCascade, NT |
| pSL1478 | pQCascade, NLS-Cas8 |
| pSL1479 | pQCascade, Cas8-T2A |
| pSL1051 | pQCascade, NLS-Cas7 |
| pSL1480 | pQCascade, Cas7-T2A |
| pSL2282 | pQCascade, NLS-Cas6 |
| pSL1053 | pQCascade, Cas6-T2A |
| pSL1419 | pQCascade, NLS-TniQ |
| pSL1477 | pQCascade, TniQ-T2A |
| pSL1483 | pTnsABC, NLS-TnsC |
| pSL1484 | pTnsABC, TnsC-T2A |
| pSL1021 | pEffector, No tags, NT |
| pSL1022 | pEffector, No tags, WT |

Plasmid construction. Genes were human codon-optimized and synthesized by Genscript, and plasmids were generated using a combination of restriction digestion, ligation, Gibson assembly, and inverted (around-the-horn) PCR. All PCR fragments for cloning were generated using Q5 DNA Polymerase (NEB).

The CRISPR array sequence (repeat-spacer-repeat) for VchINT is as follows: 5′-GTGAACTGCCGAGTAGGTAGCTGATAAC-N₃₂-GTGAACTGCCGAGTAGGTAGCTGATAAC-3′, where N₃₂ represents the 32-nt guide region.

The sequence of the mature crRNA is as follows: 5′-CUGAUAAC-N₃₂-GUGAACUGCCGAGUAGGUAG-3′.

The CRISPR array sequence (repeat-spacer-repeat) for PseINT is as follows: 5′-GTGACCTGCCGTATAGGCAGCTGAAAAT-N₃₂-GTGACCTGCCGTATAGGCAGCTGAAAAT-3′, where N₃₂ represents the 32-nt guide region.

The sequence of the mature crRNA is as follows: 5′-CUGAAAAU-N₃₂-GUGACCUGCCGUAUAGGCAG-3′.

‘Atypical’ repeats were used for PseINT (unless otherwise mentioned) to reduce the likelihood of recombination during cloning. For these variant CRISPR arrays, the repeat-spacer-repeat sequence is as follows: 5′-GTGACCTGCCGTATAGGCAGCTGAAGAT-N₃₂-TAATTCTGCCGAAAAGGCAGTGAGTAGT-3′, where N₃₂ represents the 32-nt guide region.

The sequence of the mature crRNA is as follows: 5′-CUGAAGAU-N₃₂-UAAUUCUGCCGAAAAGGCAG-3′.

E. coli culturing and general transposition assays. Chemically competent E. coli BL21(DE3) cells carrying pDonor, pDonor and pTnsABC, or pDonor and pQCascade, were prepared and transformed with 150-250 ng of pEffector, pQCascade, or pTnsABC, respectively. Transformations were plated on agar plates with the appropriate antibiotics (100 μg/ml spectinomycin, 100 μg/ml carbenicillin, 50 ng/ml kanamycin) and 0.1 mM IPTG. For bacterial transposition assays investigating PseINT activity, cells were co-transformed with pEffector and pDonor. Cells were incubated for 18-20 h at 37° C. and typically grew as densely spaced colonies, before being scraped, resuspended in LB medium, and prepared for subsequent analysis.

E. coli qPCR analysis of transposition products. The optical density of resuspended colonies from the transposition assays was measured at 600 nm, and approximately 3.2×10⁸ cells (the equivalent of 200 μl of OD600=2.0) were pelleted by centrifugation at 4,000×g for 5 min. The cell pellets were resuspended in 80 μl of H₂O, before being lysed by incubating at 95° C. for 10 min in a thermal cycler. The cell debris was pelleted by centrifugation at 4,000×g for 5 min, and 5 μl of lysate supernatant was removed and serially diluted in water to generate 20- and 500-fold lysate dilutions for qPCR analysis. Integration in the tRL orientation was measured by qPCR by comparing Cq values of a tRL-specific primer pair (one transposon- and one genome-specific primer) to a genome-specific primer pair that amplifies an E. coli reference gene (rssA). Transposition efficiency was then calculated as 2^{ΔCq}, in which ΔCq is the Cq difference between the experimental reaction and the reference reaction. qPCR reactions (10 μl) contained 5 μl of SsoAdvanced Universal SYBR Green Supermix (BioRad), 1 μl H₂O, 2 μl of 2.5 μM primers, and 2 μl of 500-fold diluted cell lysate. Reactions were prepared in 384-well clear/white PCR plates (BioRad), and measurements were performed on a CFX384 Real-Time PCR Detection System (BioRad) using the following thermal cycling parameters: polymerase activation and DNA denaturation (98° C. for 3 min), and 35 cycles of amplification (98° C. for 10 s, 59° C. for 1 min).

Mammalian cell culture and transfections. HEK293T cells were cultured at 37° C. and 5% CO₂. Cells were maintained in DMEM media with 10% FBS and 100 U/mL of penicillin and streptomycin (Fisher Scientific). The cell line was authenticated by the supplier and tested negative for mycoplasma. Cells were typically seeded at approximately 100,000 cells per well in a 24-well plate (Eppendorf or Fisher Scientific) coated with PDL (Fisher Scientific), 24 hours prior to transfection. Cells were transfected with DNA mixtures and 2 μl of Lipofectamine 2000 (Fisher Scientific), per the manufacturer's instructions.

Western imnumoblotting and nuclear cytoplasmic fractionation. Cells were transfected with epitope-tagged protein expression plasmids. Approximately 72 hours after transfection, cells were washed with PBS and harvested using Cell Lysis Buffer (150 mM NaCl, 0.1% Triton X-100, 50 mM Tris-HCl pH 8.0, Protease inhibitor (Sigma Aldrich)). For nuclear and cytoplasmic fractionation experiments, cells were harvested using Cell Lysis Buffer (Thermo Fisher Scientific) per the manufacturer's instructions. Proteins were separated by SDS-PAGE and transferred to a PVDF membrane (Fisher Scientific). The membrane was then washed with TBS-T (50 mM Tris-Cl, pH 7.5, 150 mM NaCl, 1% Tween-20) and blocked with blocking buffer (TBS-T with 5% w/v BSA). Membranes were then incubated with primary antibodies overnight at 4° C. in blocking buffer. Membranes were then washed and incubated with secondary antibodies at room temperature for one hour. Membranes were again washed and then developed with SuperSignal West Dura (Thermo Fisher).

HEK293T fluorescent reporter assays and flow cytometry analysis and sorting. HEK293T cells were seeded at approximately 50,000 cells per well in a 24-well plate coated with PDL 24 hours prior to transfection. For Cas6-mediated RNA processing assays, cells were co-transfected with 300 ng of GFP-reporter plasmid, 300 ng of Cas6 expression plasmid, and 10 ng of an mCherry expression plasmid (as a transfection marker). In negative control experiments, cells were transfected with 300 ng of a dCas9 expression plasmid instead of a Cas6 expression plasmid to control for possible expression burden or squelching. For transcriptional activation assays, cells were co-transfected with 60 ng of reporter plasmid, 20 ng of a plasmid encoding an orthogonal fluorescent protein (as a transfection marker), and the additional indicated plasmids. In separately wells, cells were transfected with 100 ng of Cas9-based transcriptional activators and 50 ng of either a non-targeting or targeting sgRNA as positive controls.

DNA mixtures were transfected using 2 μl of Lipofectamine 2000 (Fisher Scientific), per the manufacturer's instructions. Approximately 72-96 hours after transfection, cells were collected for assay by flow cytometry. Transfected cells were analyzed by gating based on fluorescent intensity of the transfection marker relative to a negative control. For assays that involved cell sorting, cells were transfected with a GFP expression plasmid and collected 4 days after transfection. A BD FACS Aria flow cytometer was used to sort cells and obtain flow cytometry data. Cells with the top 20% brightest GFP fluorescence were sorted by 5% increments into 4 bins. Cells were immediately harvested after sorting, as detailed below.

HEK293T genomic activation and RT-qPCR analysis. HEK293T cells were seeded at approximately 50,000 cells per well in a 24-well plate coated with PDL 24 hours prior to transfection. Cells were co-transfected as described above, with the following VchINT components: 100 ng pTnsABt, 50 ng pTnsC-VP64, 50 ng pTniQ, 50 ng pCas6, 250 ng pCas7, 50 ng pCas8, and 62.5 ng each of 4 targeting crRNAs for TTN, MIAT, and ASCL1 (or 83.3 ng each of 3 targeting crRNAs for ACTC1) (pCRISPR). In control experiments, cells were co-transfected with 100 ng of either pdCas9-VP64 or pdCas9-VPR plasmid, 62.5 ng each of 4 targeting sgRNAs for TTN (psgRNA), and a pUC19 plasmid to standardize transfected DNA amounts. Cells were harvested 72 hours after transfection using the RNeasy Plus Mini Kit (Qiagen), according to the manufacturer's instructions. cDNA was subsequently synthesized using the iScript cDNA Synthesis Kit (BioRad) using 1000 ng of RNA in a 20 uL reaction. Gene-specific qPCR primers were designed to amplify an approximately 180-250 bp fragment to quantify the RNA expression of each gene, and a separate pair of primers was designed to amplify ACTB (beta-actin) reference gene for normalization purposes.

qPCR reactions (10 μl) contained 5 μl of SsoAdvanced Universal SYBR Green Supermix (BioRad), 2 μl H₂O, 1 μl of 5 μM primer pair, and 2 μl of cDNA diluted 1:4 in H₂O. Reactions were prepared in 384-well white PCR plates (BioRad), and measurements were performed on a CFX384 Real-Time PCR Detection System (BioRad) using the following thermal cycling parameters: polymerase activation and DNA denaturation (98° C. for 2 min), 40 cycles of amplification (95° C. for 10 s, 60° C. for 30 s), and terminal melt-curve analysis (65-95° C. in 0.5° C. per Ss increments). Each condition was analyzed using three biological replicates, and two technical replicates were run per sample. Normalized gene activation was calculated as the ratio of the 2^{-ΔCq} of the targeting samples to the non-targeting samples, in which ΔCq is the Cq difference between the experimental gene primer pair and the reference gene primer pair.

HEK293T plasmid-to-plasmid integration assays. For assays in which plasmids were isolated and used to transform bacteria, HEK293T cells were transfected with requisite VchINT expression plasmids, a pDonor that contained a non-replicative origin of replication (R6K), a pTarget plasmid, and a crRNA expression plasmid (pCRISPR) that either encoded a non-targeting crRNA or a crRNA targeting pTarget. 72 hours after transfection, cells were thoroughly washed with PBS, harvested using TrypLE (Fisher Scientific), neutralized with culture media, and pelleted. After removal of supernatant, transfected plasmids were harvested using Qiagen Miniprep columns per the manufacturer's instructions, and further concentrated using the Qiagen MinElute column. Of this final purified plasmid mixture, 1 μl was used to electroporate NEB 10-beta electrocompetent E. coli cells (NEB) per the manufacturer's instructions. After recovery at 37° C., cells were plated onto LB-agar plates containing chloramphenicol. Chloramphenicol-resistant colonies were then replated onto LB-agar plates containing both chloramphenicol and kanamycin, and doubly-resistant colonies were harvested for genotypic analyses.

For all other integration assays, HEK293T cells were counted using a Countess 3 Cell Counter and seeded at 20,000 cells per well, unless otherwise specified, in a 24-well plate coated with PDL 24 hours prior to transfection. Cells were transfected using plasmid DNA mixtures and 2 μl of Lipofectamine 2000, per the manufacturer's instructions. For VchINT transposition assays, HEK293T cells were transfected with the following VchINT components, unless otherwise stated: 100 ng each of pTnsAB_{f}, pTnsC, pTniQ, pCas6, pCas7, pCas8, pDonor, pTarget, and 50 ng of a targeting or non-targeting crRNA (pCRISPR). For PseINT transposition assays, HEK293T cells were transfected with the following PseINT components, unless otherwise specified: 200 ng of pTnsAB, 50 ng each of pTnsC, pTniQ, pCas6, pCas7, and pCas8, 200 ng of pDonor, and 100 ng of pTarget and a targeting or non-targeting crRNA (pCRISPR).

Unless otherwise stated, cells were cultured for 4 days after transfection. Cells were washed with DPBS with no calcium or magnesium (Fisher Scientific), harvested using TrypLE (Fisher Scientific), and neutralized with culture media. 20% of the resuspended cells were pelleted by centrifugation at 300×g for 5 minutes, and the supernatant was aspirated. Cell pellets were resuspended in 50 μL of Quick Extract (Lucigen), and genomic DNA was prepared per the manufacturer's instructions.

For assays that utilized puromycin selection, HEK293T cells were transfected as described above with PseINT component plasmids and an additional 50 ng of puromycin resistance expression plasmid (as a transfection marker). Media was changed 24 hours after transfection, and selection with 1 μg/mL of puromycin was started on half of the samples. Cells were harvested using Quick Extract (Lucigen) per the manufacturer's instructions beginning at 2 days after transfection until 6 days after transfection, with or without puromycin selection. For assays that utilized cell sorting, HEK293T cells were transfected as described above with PseINT component plasmids and an additional 5 ng of GFP expression plasmid (as a transfection marker).

For assays that utilized cargo sizes ranging from 798 bp to 15 kb, HEK293T cells were transfected as described above with PseINT component plasmids, except the 5 kb, 10 kb, and 15 kb pDonor plasmids were transfected in molar equivalents to the 798 bp pDonor (˜406 fmol), to account for the size difference between donor plasmids. For assays that utilized amplicon deep sequencing, HEK293T cells were transfected as described above, with a pDonor plasmid that contained a primer binding site immediately downstream of the right transposon end that matched a primer binding site present in the unedited pTarget plasmid. Cells were harvested 4 days after transfection.

Nested PCR analysis of transposition assays. DNA amplification was performed by PCR using Q5 Hot Start High-Fidelity DNA Polymerase (NEB) following the manufacturer's protocol. In brief, 1 μL of cell lysate was added to a 25 μL PCR reaction. Thermocycling conditions were as follows: 98° C. for 45 seconds, 98° C. for 15 seconds, 66° C. for 15 seconds, 72° C. for 10 seconds, 72° C. for 2 minutes, with steps 2-4 repeated 24 times. The annealing temperature was adjusted depending on primers used. 1 μL of the first PCR reaction served as the template for a second 25 μL PCR reaction that was run under the same thermocycling conditions. Primer pairs contained one pTarget-specific primer and one transposon-specific primer, and the primers used in the second PCR reaction generated a smaller amplicon than the first reaction. PCR amplicons were resolved by 1-2% agarose gel electrophoresis and visualized by staining with SYBR Safe (Thermo Scientific). Negative control samples were always analyzed in parallel with experimental samples to identify mis-priming products, some of which presumably result from the analysis being performed on crude cell lysates that still contain the pDonor and pTarget.

qPCR analysis of plasmid-to-plasmid transposition products. Transposition-specific qPCR primers were designed to amplify a ˜140-bp fragment to quantify transposition efficiency. Primer pairs were designed to span a transposition junction, with the forward primer annealing to pTarget and the reverse primer annealing within the transposon. Additionally, a custom 5′ FAM-labeled, ZEN/3′ IBFQ probe (IDT) was designed to anneal to the plasmid-transposon junction. A separate pair of primers and a SUN-labeled, ZEN/3′ IBFQ probe (IDT) were designed to amplify a distinct segment of the target plasmid for efficiency calculation purposes.

Probe-based qPCR reactions (10 uL) contained 5 uL of Taqman Fast Advanced Master Mix, 0.5 uL of each 18 uM primer pair, 0.5 uL of each 5 uM probe, 1 uL of H₂O, and 2 uL of ten-fold diluted cell lysate. Reactions were prepared in 384-well white PCR plates (BioRad), and measurements were performed on a CFX384 Real-Time PCR Detection System (BioRad) using the following thermal cycling parameters: polymerase activation (95° C. for 10 minutes) and 50 cycles of amplification (95° C. for 15 seconds, 59.5° C. for 1 minute). Each condition was analyzed using either two or three biological replicates, and two technical replicates were run per sample. Baseline threshold ratios were manually adjusted to be 1:1 for the reference primer pair to the transposition primer pair. Transposition efficiency was calculated as a percentage as 2^{-ΔCq} times 100 in which ΔCq is the Cq difference between the reference primer pair and the transposition primer pair.

To analyze the frequency of left-right insertion (tLR) versus right-left insertion (tRL) of the PseINT transposon, transposition-specific qPCR primers were designed to span the tLR transposition junction, in addition to the primer pairs used for tRL integration and the reference amplicon in the probe-based qPCR analysis described above. qPCR reactions (10 μL) contained 5 μl of SsoAdvanced Universal SYBR Green Supermix (BioRad), 2 μl H₂O, 1 μl of 5 μM primer pair, and 2 μl of ten-fold diluted cell lysate. Reactions were prepared in 384-well white PCR plates (BioRad), and measurements were performed on a CFX384 Real-Time PCR Detection System (BioRad) using the following thermal cycling parameters: polymerase activation and DNA denaturation (98° C. for 2 min), 50 cycles of amplification (95° C. for 10 s, 59.5° C. for 20 s), and terminal melt-curve analysis (65-95° C. in 0.5° C. per 5 s increments). Each condition was analyzed using three biological replicates, and two technical replicates were run per sample.

ddPCR analysis of plasmid-to-plasmid transposition products. During harvesting of HEK293T transposition assays, 50% of the resuspended cells were reserved during lysate generation. 500 μL of resuspended cells were pelleted by centrifugation at 300×g for 5 minutes. The supernatant was aspirated, and DNA was extracted from cell pellets using the Qiagen DNeasy Blood and Tissue Kit (Qiagen). DNA was eluted in H₂O and diluted to a concentration of 2.5 ng/μL. ddPCR was performed with the same primers and probes as detailed above for plasmid-to-plasmid transposition analysis. ddPCR reactions (20 μL) contained 10 μL of ddPCR Supermix for Probes (Biorad), 1 μL of each 5 μM probe, 1 μL of each 18 μM primer pair, 5 units of HindIII (NEB), 4.13 μL of H₂O, and 2 μL of 2.5 ng/μL DNA. Reactions were assembled at room temperature, and droplets were generated using the Biorad QX200 Droplet Generator according to the manufacturer's instructions. Thermocycling was performed on a Biorad C1000 Touch Thermocycler with the following parameters: enzyme activation (95° C. for 10 minutes), 40 cycles of amplification (94° C. for 30 second, 61.5° C. for 1 minute) and enzyme deactivation (98° C. for 10 minutes). After thermocycling, droplets were hardened at 4° C. for 2 hours. Droplets were analyzed using the QX200 Droplet Reader according to the manufacturer instructions. Transposition percentages were calculated as the number of FAM positive molecules divided by the number of SUN/VIC positive molecules times 100.

Preparation of amplicons for NGS analysis. PCR-1 products were generated as described above, except primers contained universal Illumina adaptors as 5′ overhangs and the cycle number was reduced to 20. These products were then diluted 20-fold into a fresh polymerase chain reaction (PCR-2) containing indexed p5/p7 primers and subjected to 10 additional thermal cycles using an annealing temperature of 65° C. After verifying amplification by analytical gel electrophoresis, barcoded reactions were pooled and resolved by 2% agarose gel electrophoresis, DNA was isolated by Gel Extraction Kit (Qiagen), and NGS libraries were quantified by qPCR using the NEBNext Library Quant Kit (NEB). Illumina sequencing was performed using the NextSeq platform with automated demultiplexing and adaptor trimming (Illumina).

To determine the integration site distribution for a given sample, junction sequences consisting of 10-bp genomic/pTarget and 8-bp transposon end sequences were tallied for integration events 45-55 bp downstream of the PAM-distal end of the target sequence. Histograms were plotted after compiling these distances across all the reads within a given library.

### Example 14

RNA-Guided DNA Integration into Endogenous Human Genomic Target Sites

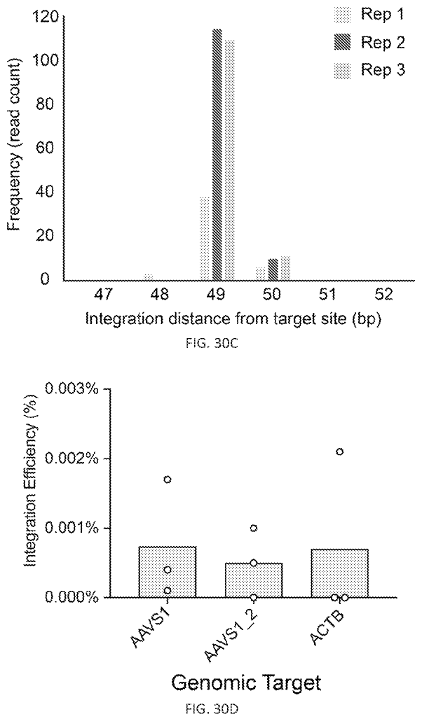

To demonstrate that RNA-guided DNA integration could be directed to target sites present endogenously in the human genome, additional guide RNAs targeting numerous genomic target sites were designed. Protein and guide RNA components were delivered via plasmid transfection, and the mini-transposon donor DNA was delivered via plasmid transfection. To verify the presence of successful integration events, and to improve the overall sensitivity for detection, a next generation sequencing (NGS) strategy was employed. Specifically, the strategy involved amplifying both the wild-type (unedited) and edited (integration-positive) alleles in a single step, such that analysis of the resulting amplicon-seq data would allow us to calculate overall integration efficiencies. To achieve this, a short sequence (approximately 20 nucleotides) was cloned within the mini-transposon on pDonor immediately inside the right transposon end; this sequence is identical to a genomic sequence downstream of the target site targeted by the CRISPR gRNA. Thus, when PCR is performed with two genome-specific primers, one primer-binding site will be present on both the unedited chromosome as well as the edited chromosome within the integrated mini-transposon, e.g., the second genome-specific primer anneals to a sequence that is present both in the donor DNA and the WT locus. With this strategy, the unedited (WT) allele and the integration-product alleles are amplified simultaneously (FIG. 30A). Using custom code for the ensuing NGS analysis, amplicons that contain a right transposon end can be differentiated from the unedited (WT) locus, integration efficiencies can be calculated, and the distance between the target site and the integration site can additionally be extracted.

Using this method, genomic integration events were reproducibly detected and quantified at a target site within the AAVS1 locus, when using a crRNA that targeted the endogenous sequence 5′-ACAGTGGGGCCACTAGGGACAGGATTGGTGAC-3′ (SEQ ID NO: 293) (FIG. 30B). When the target site distribution was analyzed, a preference for insertion events occurring 49-bp downstream of the target site was observed (FIG. 30C), similar to what has been previously observed for plasmid-to-plasmid transposition events in human cells, and for genomic transposition events in E. coli (Klompe et al., Nature 571, 219-225 (2019)).

This strategy can be broadly applied to detect integration activity at additional human genomic target sites. As expected, integration was detected and quantified at two additional target sites, including another site within the AAVS1 locus (denoted AAVS1_2) and a target site within the ACTB locus (FIG. 30D). This approach can be adopted to any additional target sites to enable highly sensitive detection and quantification of INTEGRATE-mediated transposition events.

### Example 15

### Modified Donor DNA Formulations for RNA-Guided DNA Integration

In many embodiments, the mini-transposon donor DNA is delivered to eukaryotic cells within the context of a circular DNA molecular, termed pDonor. Type I-F CRISPR-transposon systems encode the necessary enzymatic machinery to excise the mini-transposon through cleavage of both strands at both ends, via the combined action of TnsA (an endonuclease-family protein) and TusB (a DDE transposase-family protein), as was experimentally determined using long-read sequencing (Vo et al., Mob DNA 12, 13 (2021)). Because of this mechanism, the mini-transposon may also be delivered to cells within alternative contexts, since the desired genetic payload is excised through TnsA-TnsB cleavage, and the flanking (vector) DNA sequences are degraded in the cell.

In another embodiment, the mini-transposon is delivered to cells in a linear, covalently closed donor DNA form (lccDNA). This embodiment limits the amount of extraneous DNA being delivered to the cell and obviates the need to include bacterial origin and antibiotic resistance sequences that are necessary for standard plasmid cloning procedures. In addition to removing unwanted prokaryotic elements, which can enhance immunocompatibility within host eukaryotic cells, these minimized transgene vector are also smaller in size and may exhibit improved extracellular and intracellular availability, leading to improve integration (Nafissi and Slavcev. Microb. Cell Fact. 11, 154-13 (2012)). To generate lecDNA constructs, novel starting pDonor plasmids are designed and cloned, in which the mini-transposon—comprising a desired genetic payload flanked by right and left transposon end sequences, specific to the CRISPR-transposon machinery being used—is flanked on both sides with a 56-bp sequence that is recognized by the TelN protelomerase enzyme; an example of such pDonor sequence is given by SEQ ID NO: 270. Subsequently, after isolating the modified pDonor constructs from bacteria, they are incubated with the TelN enzyme (NEB), thereby generating covalently closed donor DNA. lccDNA donor molecules are separated away from unreacted pDonor and from the flanking vector backbone by gel electrophoresis, or other separation methods. The lccDNA donor molecules are then combined with standard delivery of the CRISPR-transposon protein and RNA machinery, which may be encoded by plasmids (in the case of plasmid transfection), or delivered as mRNA and gRNA, or delivered as purified protein and ribonucleoprotein complexes. lccDNA donor molecules may also be generated using alternative methods and enzymes that are standard in the field.

In other embodiments, lccDNA donor molecules are pre-complexed with the TsB transposase, such that preformed transposase-DNA co-complexes are delivered in a single step, which may be performed together with the delivery of the TniQ-Cascade complex and other transposase components (e.g., TnsA and TnsC). In other embodiments, lccDNA donor molecules are pre-complexed with the fusion TnsA-TnsB polypeptide, such that preformed transposase-DNA co-complexes are delivered in a single step; this may be performed together with the delivery of the TniQ-Cascade complex and other transposase components (e.g., TnsC). These delivery strategies, involving pre-complexing of the donor DNA with purified transposase components, may also be applied to any other donor DNA formulation, including but not limited to circular plasmid donor DNAs, lccDNA donor DNAs, simple linear donor DNAs, and linear donor DNAs with chemically modified ends. These chemically modified ends may include biotin modifications, phosphorothioate modifications, and other modifications that prevent or restrict the extent of enzymatic degradation within eukaryotic cells.

In another embodiment, mini-transposon donor DNAs are delivered to eukaryotic cells in a minimized format through the generation of minicircle DNA. Many studies have shown that minicircle DNAs can enhance transgene expression in a variety of cell types and organs, and importantly, minicircle donor DNAs also eliminate undesired prokaryotic components such as bacterial origin and antibiotic resistance sequences (Munye et al., Sci Rep 6, 23125 (2016)). Minicircle DNA substrates can also be generated in a supercoiled form. Minicircle donor DNA substrates for CRISPR-transposon based RNA-guided DNA integration applications are generated using standard methods, in which the insertion of recombination sequences flanking the mini-transposon is used, together with engineered strains of E. coli, to produce minicircles prior to the harvesting of cells and isolation of the desired DNA. The DNA may be isolated by a variety of analytical separation techniques, and the placement and identity of the recombination sequences may be optimized for greatest minicircle DNA yield, while ensuring that DNA integration activity with the CRISPR-transposon machinery is maintained within cells.

In other embodiments, minicircle donor molecules are pre-complexed with the TosB transposase, such that preformed transposase-DNA co-complexes are delivered in a single step, which may be performed together with the delivery of the TniQ-Cascade complex and other transposase components (e.g., TnsA and TnsC). In other embodiments, minicircle donor molecules are pre-complexed with the fusion TnsA-TnsB polypeptide, such that preformed transposase-DNA co-complexes are delivered in a single step; this may be performed together with the delivery of the TniQ-Cascade complex and other transposase components (e.g., TnsC).

### Example 16

### RNA-Guided DNA Integration Using Modified Guide CRISPR RNAS

Type I-F CRISPR-transposon systems typically encode CRISPR arrays that, when transcribed into pre-crRNA and then processed via the Cas6 ribonuclease, produce a 60-nucleotide RNA species containing an 8-nucleotide 5′ “handle,” a 32-nucleotide “spacer”, and a 20-nucleotide 3′ “handle” that contains a stem-loop structure. However, type I-F CRISPR-associated transposons have been shown to encode “atypical” crRNA sequences in which the 5′ and 3′ repeat sequences may encode mutations, and in which the spacer sequence is not strictly 32-nucleotides in length (Petassi et al., Cell 183, 1757-1771.e18 (2020); Klompe et al,. Mol Cell 82, 616-628.e5 (2022)). In addition, it is well known within the CRISPR field that spacer length across CRISPR arrays may be somewhat variable, depending on the CRISPR-Cas system and the CRISPR array itself, and that spacer length variation may be tolerated by the effector complexes specific to a given system.

We explored whether crRNA guides containing variable length spacer sequences would still function with PseINT, and more generally, whether alternative spacer lengths would be tolerated by CRISPR-transposon systems. It has been previously demonstrated that some variable lengths are tolerated, when increased or decreased the spacer length in 6-nt increments (Klompe et al., Nature 571, 219-225 (2019)), but here it was further investigated whether perturbations that were smaller in size would still be tolerated. Working with the PseINT system (e.g., derived from Tn7016), CRISPR arrays were generated in which the spacer contained a targeting sequence of variable length, such that the resulting mature crRNA guide would have the fixed 8-nt 5′-handle and 20-nt 3′ handle, but an intervening spacer of variable length. Within this embodiment, the spacer was varied from 20-nt to 44-nt in length, with single-nt variations tested in the length range from 30-34 (FIG. 31). Using these modified pCRISPR plasmids, RNA-guided DNA integration was tested in human cells using a plasmid-to-plasmid transposition assay, in which pDonor, pTarget, and the necessary protein and RNA expression plasmids were delivered via transfection. After culturing cells for multiple days post-transfection and then harvesting the DNA, integration was quantified using qPCR and it was found that multiple spacer lengths supported targeted, RNA-guided DNA integration. In particular, the results demonstrate that a spacer length of 33-nt functions as well, if not better, than the spacer length of 32-nt that is most commonly observed in native CRISPR arrays for Type I-F CRISPR-transposon systems (FIG. 31).

These modified crRNA guides may be used in the context of other transposition experiments, including experiments targeting human genomic sites for DNA integration. Modified crRNAs containing a 33-nt spacer may also be used for recombinant expression and purification of Cascade and/or TniQ-Cascade complexes in E. coli, such that the modified crRNA guides are delivered to mammalian cells as pre-formed, purified RNP complexes, together with the necessary transposase and donor DNA components.

### Example 17

### Streamlined Polycistronic Expression Vectors Encoding the TniQ-Cascade Complex

When investing the sensitivity of VchINT (e.g., derived from Tn6677) to the placement of epitope tags on various termini, a significant ablation of RNA-guided DNA integration activity was observed when multiple components possessed a C-terminal tag. This limited opportunities to condense the number of independent mRNA transcripts required to express the system in mammalian cells using ribosome skipping sequences known as “2A peptides.” Despite the great extent to which 2A peptides have been used in biotechnology application, the peptide that induces premature termination and reinitiation of protein synthesis on the downstream ORF remains as an obligate peptide sequence tag on the C-terminus of the upstream protein. Thus, this strategy is unavailable when upstream proteins to not tolerate C-terminal appendages.

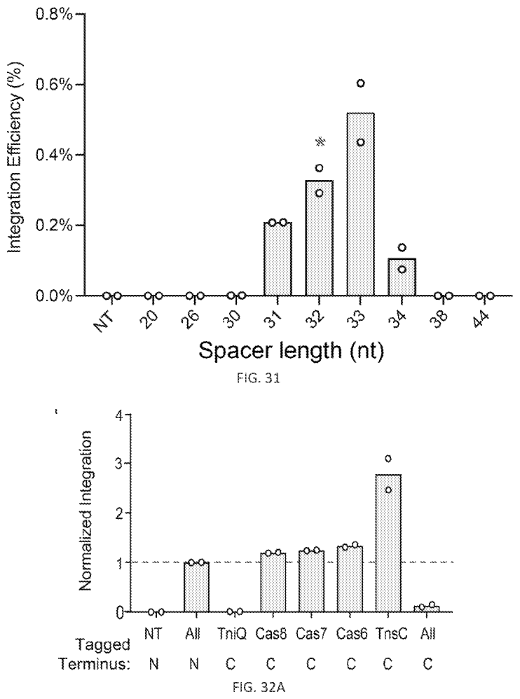

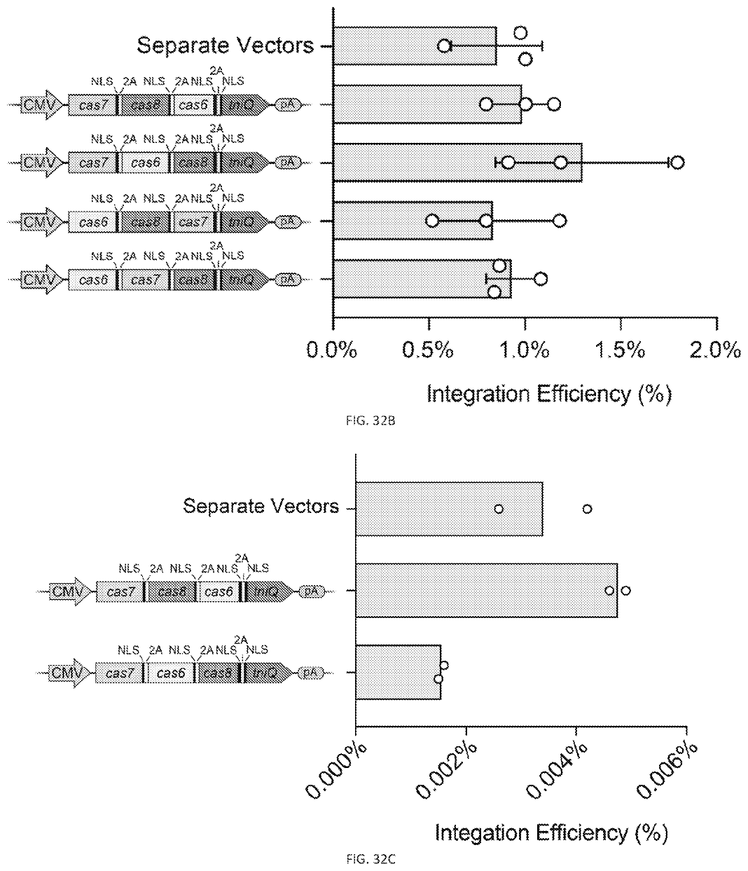

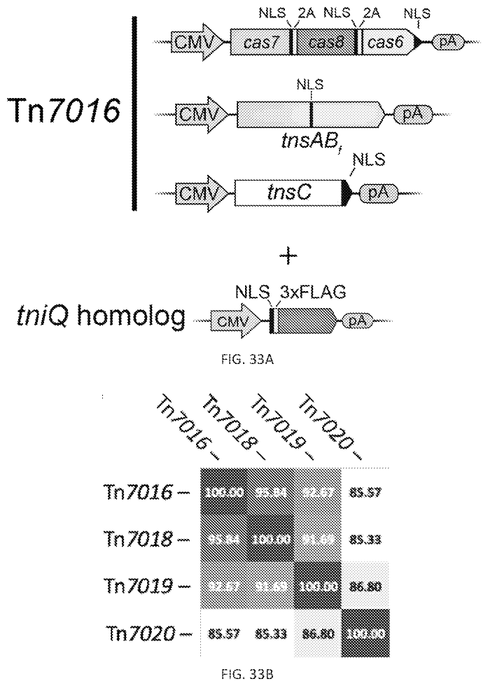

When the NLS tag sensitivity of PseINT (e.g., derived from Tn7016), which is a homologous Type I-F CRISPR-transposon system was investigated, C-terminal tags on TnsC were preferred over N-terminal tags, but that more generally, C-terminal tags were broadly tolerated across all of the protein components of the Cascade complex (e.g., Cas6, Cas7, and Cas8); however, TniQ still functioned best with an N-terminal tag, and did not tolerate C-terminal tags (FIG. 32). Thus, in certain embodiments, alternative expression vectors for the PseINT TniQ-Cascade complex were explored, in which ribosomal skipping 2A peptides were reintroduced within the context of polycistronic designs, thus allowing multiple proteins to be produced from fewer promoter-driven expression constructs. Specifically, several polycistronic vectors were designed in which all protein components of the TniQ-Cascade complex (e.g., Cas6, Cas7, Cas8, and TniQ) were encoded on a single mRNA transcript. Given the strong preference for N-terminal appendages on TniQ, all four constructs tested encoded TiQ as the final component with an N-terminal NLS tag; the remaining Cas6, Cas7, and Cas8 components were tested in various order arrangements, and in each case, contained tandem C-terminal NLS and 2A peptide tags, enabling both nuclear localization and ribosome skipping (FIG. 22.3B). Within the context of these strategies, where multiple protein-coding genes are arrayed and separated by 2A peptides, prior studies have shown that upstream protein components are generally expressed more strongly than downstream protein components (Liu et al., Sci Rep 7, 2193 (2017)).

Polycistronic vectors were screened via plasmid-to-plasmid transposition assays, in which protein and RNA expression plasmids were delivered to human cells together with pDonor and pTarget via transfection, and similar integration efficiencies were observed across all constructs, with slightly higher efficiencies when Cas7 was the first protein translated in the mRNA transcript (FIG. 32B). Genomic integration efficiencies were also investigated with polycistronic vectors encoding Cas7 first and observed higher DNA integration activity when the TniQ-Cascade complex was expressed in the order of Cas7-Cas8-Cas6-TniQ (FIG. 32C). In both plasmid- and genome-targeting DNA integration assays, the integration activity of the CRISPR-transposon systems was as high, or higher, using polycistronic vector designs for the TniQ-Cascade complex, as when each of the protein components was encoded on its own individual vector. This condensing of expression vectors reduced the number of transfected plasmids from 8 to 5 in order to carry out genomic integration.

In other embodiments, the protein components for the ToiQ-Cascade complex (e.g., TniQ, Cas6, Cas7, and Cas8) are delivered to cells via mRNA, in which the proteins may each be encoded on individual capped and polyadenylated mRNAs, or in which the proteins are similarly encoded within single capped and polyadenylated mRNAs that contain NLS and 2A peptide sequences separating each of the 4 ORF sequences.

In other embodiments, the CRISPR array may be encoded within the same polycistronic TniQ-Cascade vector, by placing an additional U6 promoter-driven element elsewhere on the plasmid. Within this embodiment, a single vector contains all the genetic instructions to express the protein and RNA components of the TniQ-Cascade complex.

In other embodiments, the CRISPR array is cloned directly within the 3′ UTR of the polycistronic vector design, optionally with stabilizing sequences upstream of the first repeat. Within this embodiment, the mature crRNA is processed directly from the capped and polyadenylated mRNA through the enzymatic action of Caso, and the stabilizing sequence upstream of the first repeat prevents rapid degradation of the protein-coding portion of the mRNA. This modified strategy allows for a single mRNA to serve as both the genetic instructions to express the protein components and guide crRNA, and thereby facilitates delivery and expression in target eukaryotic cells.

### Example 18

### Homologous CRISPR-Transposon Systems for RNA-Guided DNA Integration

As disclosed herein, PseINT, derived from Tn7016, exhibited higher RNA-guided DNA integration efficiencies in human cells when compared to VchINT, derived from Th6677. The initial set of homologs screened were highly diverse, and only sampled a small proportion of existing Type I-F CRISPR-associated transposons. In other embodiments, many other homologs are tested that are derived from this collection of potential Type I-F CRISPR-transposon systems, and these systems are screened for their ability to direct RNA-guided DNA integration activity in eukaryotic cells, either using the complete intact system, or by mixing and matching components from various systems to find a combination that optimizes expression, stability, cross-reactivity, genome-wide specificity, and integration efficiency.

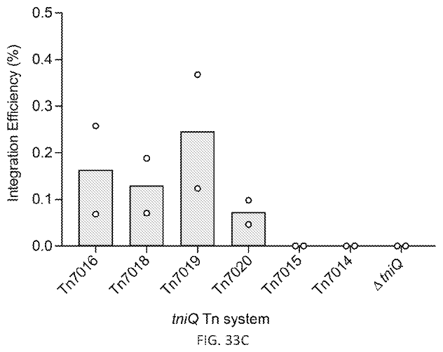

In one embodiment, additional CRISPR-transposon systems were specifically screened to investigate whether TniQ homologs would be able to function together with the other protein, RNA, and donor DNA components from PseINT (e.g., derived from Tn7016). More specifically, cells were transfected with PseINT (e.g., Tn7016) components—including a polycistronic vector encoding Cas7, Cas8, and Cas6, a vector encoding the TnsA-TnsB fusion polypeptide, a vector encoding the TnsC protein, a pCRISPR vector encoding the crRNA guide, and a pDonor vector encoding the mini-transposons—and then the system was complemented with either the cognate TniQ expression vector where the gene was derived from the same Tn70176 CRISPR-transposon system, or from a homologous CRISPR-transposon system (FIGS. 33A and 33B). These vectors were all combined with pTarget, and DNA integration was determined for plasmid-to-plasmid transposition in human cells As controls, TniQ proteins derived from Tn7015, Tn7014, and a transfection in which no TniQ was included, as all of these should exhibit no integration activity. TniQ proteins from Tn7014 and Tn7015, as well as the absence of TniQ altogether, led to a complete loss of integration activity, whereas the 3 nearby homologs tested (derived from CRISPR-associated transposons hereafter referred to Tn7018, Tn7019, and Tn7020) exhibited successful RNA-guided integration (FIG. 33C). Tn7018 is derived from Pseudoalteromonas sp. SG43-3; Tn7019 is derived from Pseudoalteromonas sp. P1-13-Ja, and Tn7020 is derived from Pseudoalteromonas arabiensis.

In other embodiments, the protein components from Tn7016 are combinatorially tested with protein, RNA, and donor DNA components from Tn7018, Tn7019, and Tn7020 in other permutations, or from other homologous CRISPR-transposon systems, in order to optimize for expression, specificity, and efficiency. In additional embodiments, structure-guided protein engineering is used to generate modified variants and/or chimeric sequences that leverage the most optimal performance of each component.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A system for RNA-guided DNA integration in a eukaryotic cell, comprising:
an engineered Clustered Regularly Interspaced Short Palindromic Repeats (CRISPR)-CRISPR associated (Cas) transposon (CRISPR-Tn) system or one or more nucleic acids encoding the engineered CRISPR-Tn system, wherein the CRISPR-Tn system comprises at least one or both of:
a) at least one Cas protein;
b) at least one transposon-associated protein; and
c) a guide RNA (gRNA) complementary to at least a portion of a target nucleic acid sequence;
wherein one or more of the at least one Cas protein and the at least one transposon-associated protein comprises a nuclear localization signal (NLS).

2. The system of claim 1, wherein one or more of the at least one Cas protein and the at least one transposon-associated protein comprises two or more NLSs.

3. The system of claim 1 or claim 2, wherein the NLS is at an N-terminus, a C-terminus, embedded in the one or more of the at least one Cas protein and the at least one transposon-associated protein or a combination thereof.

4. The system of any of claims 1-3, wherein the NLS is a monopartite sequence.

5. The system of any of claims 1-3, wherein the NLS is a bipartite sequence.

6. The system of claim 5, wherein the NLS comprises a sequence having at least 70% similarity to KRTADGSEFESPKKKRKV (SEQ ID NO:89).

7. The system of any of claim 1-6, wherein the at least one Cas protein is derived from a Type-I CRISPR-Cas system.

8. The system of any of claim 1-7, wherein the at least one Cas protein comprises Cas5, Cas6, Cas7, and Cas8.

9. The system of any of claim 1-8, wherein the at least one Cas protein comprises a Cas8-Cas5 fusion protein.

10. The system of any of claims 1-9, wherein the at least one transposon protein is derived from a Tn7 or Tn7-like transposon system.

11. The system of any of claims 1-10, wherein the at least one transposon-associated protein comprises TnsA, TnsB, TnsC, or a combination thereof.

12. The system of any of claims 1-11, wherein the at least one transposon protein comprises a TnsA-TnsB fusion protein.

13. The system of claim 12, wherein the TnsA-TnsB fusion protein further comprises an amino acid linker between TnsA and TnsB.

14. The system of claim 13, wherein the linker is a flexible linker.

15. The system of claim 13 or claim 14, wherein the linker comprises at least one glycine-rich region.

16. The system of any of claims 13-15, wherein the linker comprises a NLS sequence.

17. The system of claim 16, wherein the linker comprises a NLS sequence flanked on each end by a glycine rich region.

18. The system of any of claims 1-17, wherein the at least one transposon-associated protein comprises TnsD and/or TniQ.

19. The system of any of claims 1-18, wherein the CRISPR-Tn system is derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

20. The system of any of claims 1-19, wherein the at least one gRNA is a non-naturally occurring gRNA.

21. The system of any of claims 1-20, wherein the at least one gRNA is encoded in a CRISPR RNA (crRNA) array.

22. The system of any of claims 1-21, wherein the gRNA is transcribed under control of an RNA Polymerase II promoter or RNA Polymerase III promoter.

23. The system of any of claims 1-22, wherein the one or more nucleic acids comprises one or more messenger RNAs, one or more vectors, or a combination thereof.

24. The system of any of claims 1-23, wherein the at least one Cas protein, the at least one transposon-associated protein, and the gRNA are encoded by different nucleic acids.

25. The system of any of claims 1-23, wherein one or more of the at least one Cas protein, the at least one transposon-associated protein, and the gRNA are encoded by a single nucleic acid.

26. The system of claim 24 or claim 25, wherein Cas7 is encoded by an individual nucleic acid.

27. The system of claim 25, wherein a single nucleic acid encodes the gRNA and at least one Cas protein.

28. The system of claim 27, wherein the at least one Cas protein is Cas6 or Cas7.

29. The system of any of claims 8-28, wherein the system comprises Cas7 or the nucleic acid encoding Cas7 in greater abundance compared to the remaining protein components or nucleic acids encoding thereof.

30. The system of claim 29, wherein each of the at least one Cas protein, the at least one transposon-associated protein, and the gRNA are encoded by a single nucleic acid.

31. The system of any of claims 1-30, wherein the one or more nucleic acids further comprise or encode a sequence capable of forming a triple helix downstream of the sequence encoding the at least one Cas protein or the sequence encoding the at least one transposon-associated protein.

32. The system of claim 31, wherein the sequence capable of forming a triple helix is in a 3′ untranslated region of the sequence encoding the at least one Cas protein or the sequence encoding the at least one transposon-associated protein.

33. The system of any of claims 1-32, wherein one or more of the nucleic acid encoding at least one Cas protein and the nucleic acid at least one transposon-associated protein comprises a sequence encoding a ribosome skipping peptide.

34. The system of claim 33, wherein the ribosome skipping peptide comprises a 2A family peptide.

35. The system of any of claims 1-34, wherein each of the at least one Cas protein and the at least one transposon-associated protein are part of a single fusion protein.

36. The system of any of claims 1-35, wherein one or more of the at least one Cas protein are part of a ribonucleoprotein complex with the gRNA.

37. The system of any of claims 1-36, further comprising a donor nucleic acid to be integrated, wherein said donor DNA comprises a cargo nucleic acid sequence flanked by at least one transposon end sequence.

38. A system for DNA integration into a target nucleic acid sequence comprising:
an engineered Clustered Regularly Interspaced Short Palindromic Repeats (CRISPR)-CRISPR associated (Cas) transposon (CRISPR-Tn) system or one or more nucleic acids encoding the engineered CRISPR-Tn system, wherein the CRISPR-Tn system comprises at least one or both of:
a) at least one Cas protein; and
b) TnsA, TnsB, TnsC, or a combination thereof,
wherein the engineered CRISPR-Tn system is derived from Vibrio parahaemolyticus, Aliibrio sp., Pseudoalteromonas sp., or Endozoicomonas ascidiicola.

39. The system of claim 38, wherein the engineered CRISPR-Tn system is a Type I-F system.

40. The system of claim 38 or claim 39, wherein the engineered CRISPR-Tn system is a Type I-F3 system.

41. The system of any of claims 38-40, wherein the one or more nucleic acids comprises one or more messenger RNAs, one or more vectors, or a combination thereof.

42. The system of any of claims 38-41, wherein the at least one Cas protein and the TnsA, TosB, and TnsC are encoded by different nucleic acids.

43. The system of any of claims 38-41 wherein the at least one Cas protein and the TnsA, TnsB, and TnsC are encoded by a single nucleic acid.

44. The system of any of claims 38-43, wherein the engineered CRISPR-Tn system further comprises TnsD, TniQ, or a combination thereof or a nucleic acid encoding TnsD, TniQ, or a combination thereof.

45. The system of any of claims 38-44, wherein the at least one Cas protein comprises Cas5, Cas6, Cas7, and Cas8.

46. The system of any of claims 38-45, wherein the at least one Cas protein comprises Cas8-Cas5 fusion protein.

47. The system of any of claims 38-46, wherein the engineered CRISPR-Tn system comprises Cas5, Cas6, Cas7, Cas8, TnsA, TnsB, TnsC, and at least one or both of TnsD or TniQ.

48. The system or kit of any of claims 38-47, wherein the engineered CRISPR-Tn system comprises TnsA, TnsB, TnsC, TnsD and TniQ.

49. The system of any of claims 46-48, wherein the system comprises Cas7 or a nucleic acid encoding Cas7 in greater abundance compared to the remaining protein components or nucleic acids encoding thereof.

50. The system of any of claims 38-49, wherein one or more of the at least one Cas protein, TnsA, TosB, TasC, TnsD, and TniQ comprises a nuclear localization signal (NLS).

51. The system of any of claims 38-50, wherein one or more of the at least one Cas protein, TnsA, TnsB, TnsC, TnsD, and TniQ comprises two or more NILSs.

52. The system of claim 50 or claim 51, wherein the NLS is at an N-terminus, a C-terminus, embedded in the at least one Cas protein, TnsA, TnsB, TnsC, TnsD, and TniQ, or a combination thereof.

53. The system of any of claims 38-52, wherein TnsA and TnsB are provided as a TnsA-TnsB fusion protein.

54. The system of claim 53, wherein the TnsA-TnsB fusion protein further comprises an amino acid linker between TnsA and InsB.

55. The system of claim 54, wherein the linker is a flexible linker.

56. The system of claim 54 or claim 55, wherein the linker comprises at least one glycine-rich region.

57. The system of any of claims 54-56, wherein the linker comprises a nuclear localization signal (NLS).

58. The system of claim 57, wherein the linker comprises a NLS flanked on each end by a glycine rich region.

59. The system of any of claims 50-58, wherein the NLS is a monopartite sequence.

60. The system of claim 59, wherein the NLS is a bipartite sequence.

61. The system of claim 59 or claim 60, wherein the NLS comprises a sequence having at least 70% similarity to KRTADGSEFESPKKKRKV (SEQ ID NO:89).

62. The system of any of claims 38-61, wherein the engineered CRISPR-Tn system further comprises at least one gRNA complementary to at least a portion of the target nucleic acid sequence, or a nucleic acid encoding the at least one gRNA.

63. The system of claim 62, wherein the at least one gRNA is encoded by a nucleic acid different from the nucleic acid(s) encoding the at least one Cas protein and TnsA, TnsB, and TnsC.

64. The system of claim 62, wherein the at least one gRNA is encoded by a nucleic acid also encoding the at least one Cas protein, TnsA, TnsB, and TnsC, or both.

65. The system of any of claims 62-64, wherein the at least one gRNA is a non-naturally occurring gRNA.

66. The system of any of claims 62-65, wherein the at least one gRNA is encoded in a CRISPR RNA (crRNA) array.

67. The system of any of claims 38-66, wherein the one or more nucleic acids further comprise or encode a sequence capable of forming a triple helix downstream of the sequence encoding the engineered CRISPR-Tn system.

68. The system of claim 67, wherein the sequence capable of forming a triple helix is in a 3′ untranslated region of the sequence encoding the at least one Cas protein or the sequence encoding at least one of TnsA, TnsB, TnsC, TnsD, and TniQ.

69. The system of any of claims 38-68, wherein one or more of the nucleic acids encoding the engineered CRISPR-Tn system comprises a sequence encoding a ribosome skipping peptide.

70. The system of claim 69, wherein the ribosome skipping peptide comprises a 2A family peptide.

71. The system of any of claims 38-70, further comprising a target nucleic acid sequence.

72. The system of claim 71, wherein the target nucleic acid sequence comprises a TnsD binding site.

73. The system of claim 71 or claim 72, wherein the target nucleic acid sequence comprises a human nucleic acid sequence.

74. The system of any of claims 38-73, further comprising a donor nucleic acid flanked by at least one transposon end sequence.

75. The system of kit of claim 74, wherein the donor nucleic acid comprises a human nucleic acid sequence.

76. The system or kit of claim 74 or claim 75, wherein the nucleic acid encoding the at least one Cas protein, TnsA, TnsB, and TnsC, the at least one gRNA, or any combination thereof further comprises the donor nucleic acid.

77. A system for RNA-guided DNA integration in a eukaryotic cell, comprising:
an engineered Clustered Regularly Interspaced Short Palindromic Repeats (CRISPR)-CRISPR associated (Cas) transposon (CRISPR-Tn) system or one or more nucleic acids encoding the engineered CRISPR-Tn system, wherein the CRISPR-Tn system comprises at least one or both of:
a) at least one Cas protein comprising Cas7;
b) at least one transposon-associated protein; and
c) a guide RNA (gRNA) complementary to at least a portion of a target nucleic acid sequence;
wherein the system comprises Cas7 or the nucleic acid encoding Cas7 in greater abundance compared to the remaining protein components or nucleic acids encoding thereof.

78. The system of claim 77, wherein one or more of the at least one Cas protein and the at least one transposon-associated protein comprises a nuclear localization signal (NLS).

79. The system of claim 77, wherein one or more of the at least one Cas protein and the at least one transposon-associated protein comprises two or more NLSs.

80. The system of claim 78 or claim 79, wherein the NLS is appended to the one or more of the at least one Cas protein and the at least one transposon-associated protein at a N-terminus, a C-terminus, or a combination thereof.

81. The system of any of claims 78-80, wherein the NLS is a monopartite sequence.

82. The system of any of claims 78-80, wherein the NLS is a bipartite sequence.

83. The system of claim 82, wherein the NLS comprises a sequence having at least 70% similarity to KRTADGSEFESPKKKRKV (SEQ ID NO:89).

84. The system of any of claim 77-83, wherein the at least one Cas protein is derived from a Type-I CRISPR-Cas system.

85. The system of any of claim 77-84, wherein the at least one Cas protein comprises Cas5, Cas6, Cas7, and Cas8.

86. The system of claim 85, wherein the at least one Cas protein comprises a Cas8-Cas5 fusion protein.

87. The system of any of claims 77-86, wherein the at least one transposon protein is derived from a Tn7 or Tn7-like transposon system.

88. The system of any of claims 77-87, wherein the at least one transposon-associated protein comprises TnsA, TnsB, and TnsC.

89. The system of any of claims 77-88, wherein the at least one transposon protein comprises a TnsA-TnsB fusion protein.

90. The system of claim 89, wherein the TnsA-TnsB fusion protein further comprises an amino acid linker between TnsA and TnsB.

91. The system of claim 90, wherein the linker is a flexible linker.

92. The system of claim 90 or claim 91, wherein the linker comprises at least one glycine-rich region.

93. The system of any of claims 90-92, wherein the linker comprises a NLS sequence.

94. The system of claim 93, wherein the linker comprises a NLS sequence flanked on each end by a glycine rich region.

95. The system of any of claims 77-94, wherein the at least one transposon-associated protein comprises TnsD and/or TniQ.

96. The system of any of claims 77-95, wherein the CRISPR-Tn system is derived from Vibrio cholerae, Photobacterium iliopiscarium, Vibrio parahaemolyticus, Pseudoalteromonas sp., Pseudoalteromonas ruthenica, Photobacterium ganghwense, Shewanella sp., Vibrio diazotrophicus, Vibrio sp. 16, Vibrio sp. F12, Vibrio splendidus, Aliivibrio wodanis, Aliivibrio sp., Endozoicomonas ascidiicola, and Parashewanella spongiae.

97. The system of any of claims 77-96, wherein the at least one gRNA is a non-naturally occurring gRNA.

98. The system of any of claims 77-97, wherein the at least one gRNA is encoded in a CRISPR RNA (crRNA) array.

99. The system of any of claims 77-98, wherein the gRNA is transcribed under control of an RNA Polymerase II promoter.

100. The system of any of claims 77-99, wherein the one or more nucleic acids comprises one or more messenger RNAs, one or more vectors, or a combination thereof.

101. The system of any of claims 77-100, wherein the at least one Cas protein, the at least one transposon-associated protein, and the gRNA are encoded by different nucleic acids.

102. The system of any of claims 77-100, wherein one or more of the at least one Cas protein, the at least one transposon-associated protein, and the gRNA are encoded by a single nucleic acid.

103. The system of claim 101 or claim 102, wherein Cas7 is encoded by an individual nucleic acid.

104. The system of claim 100, wherein a single nucleic acid encodes the gRNA and at least one Cas protein.

105. The system of claim 104, wherein each of the at least one Cas protein, the at least one transposon-associated protein, and the gRNA are encoded by a single nucleic acid.

106. The system of any of claims 77-105, wherein the one or more nucleic acids further comprise or encode a sequence capable of forming a triple helix downstream of the sequence encoding the at least one Cas protein or the sequence encoding the at least one transposon-associated protein.

107. The system of claim 106, wherein the sequence capable of forming a triple helix is in a 3′ untranslated region of the sequence encoding the at least one Cas protein or the sequence encoding the at least one transposon-associated protein.

108. The system of any of claims 77-107, wherein one or more of the nucleic acid encoding at least one Cas protein and the nucleic acid at least one transposon-associated protein comprises a sequence encoding a ribosome skipping peptide.

109. The system of claim 108, wherein the ribosome skipping peptide comprises a 2A family peptide.

110. The system of any of claims 77-109, wherein each of the at least one Cas protein and the at least one transposon-associated protein are part of a single fusion protein.

111. The system of any of claims 77-110, wherein one or more of the at least one Cas protein are part of a ribonucleoprotein complex with the gRNA.

112. The system of any of claims 77-111, further comprising a donor nucleic acid to be integrated, wherein said donor DNA comprises a cargo nucleic acid sequence flanked by at least one transposon end sequence.

113. The system of any of claims 1-112, wherein the system is a cell-free system.

114. A composition comprising the system of any of claims 1-113.

115. A cell comprising the system of any of claims 1-112.

116. The cell of claim 115, wherein the cell is a prokaryotic cell.

117. The cell of claim 115, wherein the cell is a eukaryotic cell.

118. The cell of claim 117, wherein the cell is a mammalian cell.

119. The cell of claim 117 or claim 118, wherein the cell is a human cell.

120. A method for DNA integration comprising contacting a target nucleic acid sequence with the system of any of claims 1-112 or a composition of claim 114.

121. The method of claim 120, wherein the target nucleic acid sequence is in a cell.

122. The method of claim 121, wherein the contacting a target nucleic acid sequence comprises introducing the system into the cell.

123. The method of claim 122, wherein the cell is a prokaryotic cell.

124. The method of claim 123, wherein the cell is a eukaryotic cell.

125. The method of claim 124, wherein the cell is a mammalian cell.

126. The method of claim 124 or claim 125, wherein the cell is a human cell.

127. The method of any of claims 122-126, wherein the introducing the system into the cell comprises administering the system to a subject.

128. The method of claim 127, wherein the administering comprises in vivo administration.

129. The method of claim 127, wherein the administering comprises transplantation of ex vivo treated cells comprising the system.

130. Use of the system of any of claims 1-112 or a composition of claim 114 for integrating DNA into a target nucleic acid sequence.

131. The use of claim 130, wherein the target nucleic acid sequence is in a cell.

132. The use of claim 131, wherein the contacting a target nucleic acid sequence comprises introducing the system into the cell.

133. The use of claim 132, wherein the cell is a prokaryotic cell.

134. The use of claim 132, wherein the cell is a eukaryotic cell.

135. The use of claim 134, wherein the cell is a mammalian cell.

136. The use of claim 134 or claim 135, wherein the cell is a human cell.
